(19) United States
(12) Patent Application Publication
(10) Pub. No.: US 2011/0293540 A1
(43) Pub. Date: Dec. 1, 2011

(54) BRANCHED POLYMERS, COMPOSITIONS, AND USES THEREOF

(75) Inventors: Osama M. Musa, Kinnelon, NJ (US); David C. Streuli, Wayne, NJ (US); Jose Martinez-Santiago, New York, NY (US); Cuiyue Lei, Wayne, NJ (US)

(73) Assignee: ISP INVESTMENTS INC., Wilmington, DE (US)

(21) Appl. No.: 13/178,224

(22) Filed: Jul. 7, 2011

Related U.S. Application Data
(63) Continuation-in-part of application No. PCT/US11/20208, filed on Jan. 5, 2011.
(60) Provisional application No. 61/292,897, filed on Jan. 7, 2010.

(51) Int. Cl. A61K 8/81; C08F 122/06; C08F 26/06; A61Q 5/06; C08F 130/08; C09D 161/00; A61Q 17/04; A61Q 11/00; C08F 26/00; C08F 128/02

(52) U.S. Cl. 424/49; 526/312; 526/271; 526/307.4; 526/263; 526/286; 526/279; 526/264; 524/555; 424/70.11; 424/59

(57) ABSTRACT

Provided herein are branched polymers synthesized from: at least one (A1) reactive monomer having a vinyl functional group, or (A2) a hybrid reactive monomer having (a) at least one vinyl functional group and (b) at least one reactive non-vinyl functional group; and at least one: (B1) branching agent having at least two N-vinyl formamide functional groups or (B2) a hybrid branching agent having (a) at least one N-vinyl formamide functional group, and (b) at least one vinyl functional group. The polymers exhibit branching behavior, which in some embodiments lends to lower viscosity, enhanced processability, and/or improved sprayability compared to a corresponding polymer of equal molecular weight synthesized without the second reactive moiety. Formulations with the invention's polymers also are presented.

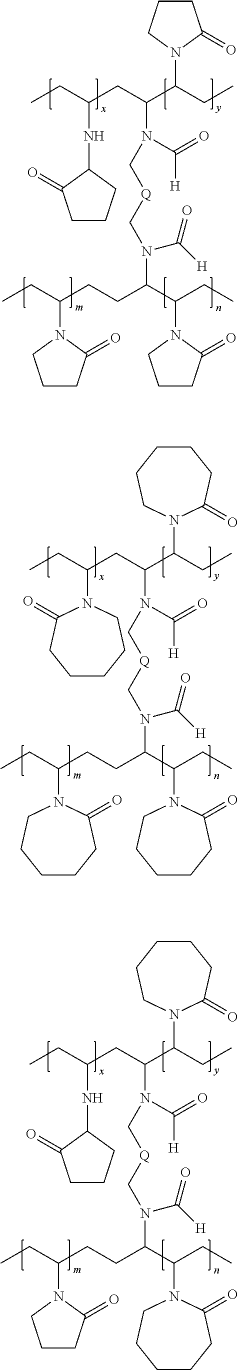

### DESCRIPTION OF RELATED ART

The present invention relates to branched polymers, their properties, compositions and uses thereof.

N-vinyl amides are electron rich monomers. Commonly known cyclic N-vinyl amides include N-vinyl-2-pyrrolidone (VP) and N-vinyl-2-caprolactam (VCL), and commonly known acyclic N-vinyl amides are N-vinyl acetamide (NVA) and N-vinyl formamide (NVF). Likewise, the lactam polymers, including poly(N-vinyl-2-pyrrolidone) (PVP), are well known, as described for example in the book by B. V. Robinson et al., “PVP: A Critical Review of the Kinetics and Toxicology of Polyvinyl pyrrolidone (Povidone)”, 1990, Lewis Publishers, Inc., Chelsea, Mich.; and in U.S. Pat. Nos. 3,153,640, 2,927,913, 3,532,679; and GB patent 811,135. PVP has been used extensively in many arts, ranging from coatings to pharmaceuticals to personal care. The toxicity of PVP has been studied extensively in a variety of species, including humans and other primates, and is extremely low.

The synthesis of N-vinyl amides can be accomplished through the vinylation reaction of amide through addition to acetylene, or through a trans-vinylation reaction with vinyl ether or vinyl acetate. N-vinyl amides can also be prepared by cracking a vinyl amide precursor. The synthesis of multifunctional N-vinyl amide compounds can proceed through a C-alkylation reaction using a lithium base, or through the use of an N-alkylation reaction requiring the use of sodium hydride (NaH), which is typically not preferred in industrial manufacturing environments. In another method, N-vinyl acetamide can be de-protonated by NaOH in the presence of a phase transfer catalyst to create difunctional monomers that can be used to make polymers with cyclic backbones. Michael addition of N-vinyl formamides to acrylonitrile and to acrylates and methacrylates has been used for the synthesis of N-cyanoethyl-N-vinyl-formamide and 3-(N-vinylformamido)propionates, respectively. In both cases, the synthesis was focused on monofunctional substituted N-vinyl amides. The synthetic routes disclosed above relate to either multifunctional N-vinyl acetamide or N-vinyl pyrrolidone, or to monofunctional N-vinyl formamides.

There is a desire, however, to extend the performance of polymers beyond what is currently known. In many applications polymers are needed that exhibit low viscosity while maintaining benefits of a higher molecular weight. Such polymers could be used in spraying, pumping, and mixing applications that currently are limited by high viscosity. Likewise, there is a demand for polymers that offer better processability (like blendability, mixability, pumpability, and/or extrudability) to enable entirely new formulations that cannot otherwise be created. Examples of limitations may include high equipment torque, motor failure, and/or product inhomogeneity. One approach to this problem is to synthesize branched polymer variants. One approach to creating these branched polymer variants is to use branching agents.

Branching agents are moieties that have the functionality to link one monomer and/or polymer chain to another monomer and/or polymer chain, and result in polymers that are non-linear polymers, or partially crosslinked polymers, wherein either the non-linear and/or partially crosslinked polymers may contain linear polymer portion(s), and combinations thereof. Branching agents can be designed with different spacer arm lengths between the reactive groups, which helps to modulate the final product properties. Branching agents with short space arms may be suitable for intramolecular bonding. In contrast, intermolecular bonding between distant molecules can be attained by increasing the spacer arm length.

Among other factors, the resulting polymer properties can depend on the selected monomer(s), branching agent(s) (with its spacer arm length), and the branching agent addition level and the resulting polymer chain densities. Low addition levels of these agents give rise to branched polymer behavior, meaning that the polymer does not exhibit properties typical of polymers made without the branching agent. Branched polymers may exhibit a lower viscosity than linear polymers of equal molecular weight made without the branching agent, an effect attributed in part to fewer polymer chain entanglement arising from the branched polymer architecture. Because they lack extensive covalent bonds between polymer chains (which is achieved at high branching agent addition levels), a branched polymer exhibits a solubility in the same solvents that solubilize the analogous polymer of equal molecular weight but synthesized without the branching agent(s). As just mentioned, with further increases in the branching agent(s), the resulting polymer can display networked hydrogel behavior, which may include elastic properties such as increased elongational viscosity and/or higher strengths. At high addition levels crosslinked polymers may be created, becoming very rigid or glassy, such as phenol-formaldehyde materials.

Disclosures discussing crosslinkers and N-vinyl formamides include WO 2009/099436, WO 2007/096400, WO 2008/032342, and U.S. Pat. Nos. 5,300,606; 5,338,815; 5,534,174 and 5,788,950.

Disclosures of branched polymers, especially poly(N-vinyl-2-pyrrolidone), are provided in U.S. Pat. Nos. 5,082,910; 5,159,034; and 6,294,064. The '910 and '034 inventions provide processes for preparing linear polymers of high molecular weight, as contrasted to branched polymers of low molecular weight. Also known are poly(N-vinyl-2-pyrrolidone)-b-polyester, as disclosed on U.S. patent application 2008/0262105.

Also related are U.S. Pat. Nos. 4,774,285; 5,534,174; and 5,569,725. Water-soluble copolymers produced from 95 to 10 mol % N-vinyl formamide and 5 to 90 mol % vinyl acetate and/or vinyl propionate are claimed in the '285 patent. The '174 patent relates to grafted copolymers comprising a star-shaped polymer and an N-allyl amide. N-vinyl amide terminated resins are the subject of the '725 patent. Lastly, a method for synthesizing N-vinyl formamide derivatives is taught in the U.S. Pat. No. 7,135,598.

Note is made that some publications refer to “branched polyvinylpyrrolidone”, such as described in U.S. patent application 2008/0031946. This material is a fully crosslinked, water-swellable, but water-insoluble polymer (i.e., Polyplasdone® sold into commercial sale by International Specialty Products, ISP), and such a definition of “branched polyvinylpyrrolidone” is not shared with the present invention. In other publications (e.g., U.S. Pat. No. 6,465,692) use the terms “branched polyvinylpyrrolidone” or “branched PVP”, but are silent as to what is meant (i.e., water soluble or fully crosslinked and water-insoluble).

Related disclosure includes international patent application PCT/US11/20208, which is hereby incorporated in its entirety by reference. That application provides polymers resulting from the polymerization of at least one reactive vinyl monomer moiety and a multifunctional N-vinyl formamide crosslinking moiety. Examples of polymers include those polymerized using one of the following multifunctional N-vinyl formamide crosslinking moieties:

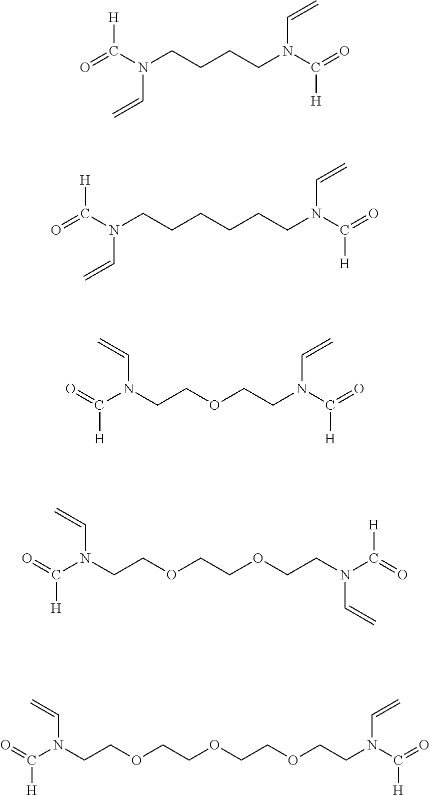

The '208 application also discloses polymers resulting from the polymerization of at least one reactive vinyl monomer and a hybrid N-vinyl formamide crosslinking moiety having at least one N-vinyl formamide functionality and at least one other reactive vinyl functionality. For example, polymers include those polymerized using one of the following hybrid N-vinyl formamide moieties:

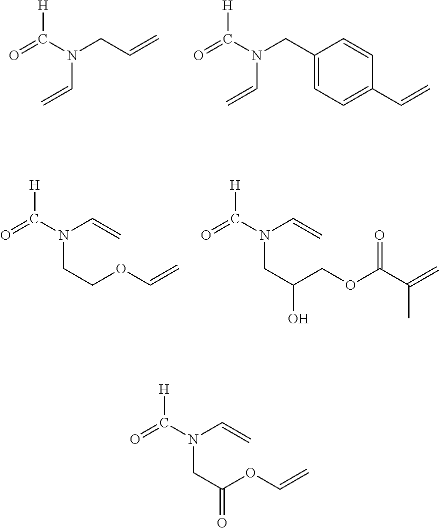

Hence, one aim of the invention to provide branched polymers that demonstrate lower viscosity, enhanced processability, and/or improved sprayability. Another aim is to illustrate non-limiting formulations and uses of these polymers to the personal care arts.

### SUMMARY OF THE INVENTION

Provided herein is a new class of branched polymers, being synthesized from at least one reactive monomer and at least one branching agent. There are at least four broad classes of polymers that arise from two types of reactive monomers and two types of branching agents. The reactive monomer is a reactive monomer having a vinyl functional group, or a hybrid reactive monomer having (a) at least one vinyl functional group and (b) at least one reactive non-vinyl functional group, or combinations thereof. The branching agent is a branching agent having at least two N-vinyl formamide functional groups, or a hybrid branching agent having (a) at least one N-vinyl formamide functional group and (b) at least one vinyl functional group, or combinations thereof.

The invention includes embodiments wherein the branched polymers exhibit lower viscosity, enhanced processability, and/or improved sprayability compared to a corresponding polymer of equal molecular weight synthesized without the second reactive moiety. The branched polymers find broad application in numerous formulations, especially those where the benefits of a high molecular weight at lower viscosity are useful. Examples of such formulations include, but at not limited to, the personal care compositions (e.g., hair care, hair styling, skin care, oral care, sun care, color cosmetic), coatings, adhesives, cleaning compositions, detergents, oilfield arts, membranes, and pigment dispersions.

### BRIEF DESCRIPTION OF THE FIGURES

FIG. 1 is a graph of log(intrinsic viscosity) as a function of log(weight-average molecular weight) as discussed in accordance with Example 62.

FIG. 2 is a graph of molecular weight and K-value as a function of DVFOO addition level as described in accordance with Example 63.

FIG. 3 is a graph of weight-average molecular weight as a function of retention volume for polymers discussed in accordance with Example 64.

FIG. 4 is a graph of droplet size distribution frequency for a mist prepared in accordance with Example 66.

FIG. 5 is a graph of droplet size distribution frequency for a mist prepared in accordance with Example 66.

FIG. 6 is a graph of droplet size distribution frequency for a mist prepared in accordance with Example 67.

FIG. 7 is a graph of droplet size distribution frequency for a mist prepared in accordance with Example 68.

FIG. 8 is a graph of droplet size distribution frequency for a mist prepared in accordance with Example 69.

FIGS. 9A-9D are graphs of spray patterns produced in accordance with Example 71.

FIG. 10 is a graph of average percent curl remaining as a function of time for three formulas produced in accordance with Example 72.

### DETAILED DESCRIPTION

The invention has proved especially versatile in providing branched polymers that may show properties distinctly apart from their unbranched counterparts, or their fully crosslinked analogues. Also described herein are formulations utilizing these branched polymers. The reader understands these embodiments and particulars used to describe them do not limit the scope of the invention.

The branched polymers result from the polymerization of at least one reactive monomer selected from the group consisting of (A1) a reactive monomer having a vinyl functional group, (A2): a hybrid reactive monomer having (a) at least one vinyl functional group and (b) at least one reactive, non-vinyl functional group, and combinations thereof. The polymerization of the reactive monomer(s) proceeds in the presence of at least one branching agent selected from the group consisting of: (B1): a branching agent having at least two N-vinyl formamide functional groups, and (B2): a hybrid branching agent having (a) at least one N-vinyl formamide functional group and (b) at least one vinyl functional group, and combinations thereof.

In one embodiment at least 10% (w/w) of the branched polymer is soluble in a solvent that solubilizes the corresponding polymer of equal molecular weight made without the (B1) or (B2) branching agent. In other embodiments the branched polymers exhibit lower viscosity, enhanced processability, and/or improved sprayability compared to a corresponding polymer of equal molecular weight synthesized without the (B1) or (B2) branching agent.

Also provided are formulations that may advantageously utilize the branched polymers' properties. Examples of these formulations include adhesives, aerosols, agricultural compositions, beverages, cleaning compositions, coating compositions, dental compositions, detergents, drugs, encapsulations, foods, hair sprays, lithographic solutions, membrane formulations, oilfield formulations, personal care compositions, pharmaceuticals, pigment dispersions, and the like. Personal care compositions refers to such illustrative non-limiting compositions as skin, sun, hair, oral, cosmetic, and preservative compositions, including those to alter the color and appearance of the skin. Other personal care compositions include, but are not limited to, polymers for increased flexibility in styling, durable styling, increased humidity resistance for hair, skin, and color cosmetics, sun care water-proof/resistance, wear-resistance, and thermal protecting/enhancing compositions. Dental personal care compositions include denture adhesives, toothpastes, mouthwashes, chewing gums, tooth whiteners, stain removers, and the like that can deliver an active ingredient (such as an anti-gingivitis active and/or a breath freshening active). Pharmaceutical compositions include tablet coatings, tablet binders, transdermal patches, and the like.

Before describing the polymers, compositions, and methods, the following definitions are provided:

As setout above, commonly known electron rich N-vinyl amides include, but are not limited to, the following species:

N-vinyl acetamide (NVA):

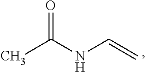

N-vinyl-2-caprolactam (VCL):

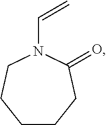

N-vinyl formamide (NVF):

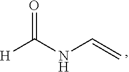

and N-vinyl-2-pyrrolidone (VP):

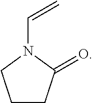

The term “allyls” refers to moieties comprising at least one allyl group.

The term “branched polymer” refers to any non-linear polymer, or partially crosslinked polymer, wherein either the non-linear and/or partially crosslinked polymer may contain linear polymer portion(s), and combinations thereof. The term “branched polymer” does not refer to a 100% completely linear polymer. In certain embodiments, these branched polymers are at least 10% soluble in the same solvent that solubilizes the corresponding polymer of equal molecular weight made without the branching agent(s) [denoted (B1) and (B2) herein]. Other embodiments recognize that branched polymers may exhibit lower viscosity, enhanced processability, and/or improved sprayability compared to the aforementioned linear polymer of equal molecular weight synthesized without the branching agent.

The term “branching agent” refers to molecules that can link one monomer and/or polymer to another monomer and/or polymer chain. Within the scope of this invention, the term “branching agent” refers to: (B1) branching agents having at least two N-vinyl formamide functional groups, and (B2) hybrid branching agents having (a) at least one N-vinyl formamide functional group and (b) at least one vinyl functional group.

The term “(meth)acrylate” refers to both acrylate and methacrylate. Similarly, the term “(meth)acrylamide” refers to both acrylamide and (meth)acrylamide.

The term “polymer” refers to a large molecule (macromolecule) comprising repeating structural units (monomers) connected by covalent chemical bonds.

The term “reactive monomer” refers to monomers having moiety(ies) that can react with the (B1) and (B2) branching agents. Non-limiting illustrative examples these moieties include anhydrides, vinyl amides, acrylates, styrenes, maleimides, maleates, fumarates, cinnamyls, vinyl imidazoles, vinyl pyridines, vinyl acetates, acrylamides, vinyl sulfones, vinyl carbonates, vinyl silanes, vinyl acrylamides, allyls, vinyl ethers, epoxides, oxetanes, benzoxazines, oxazolines, and mixtures thereof.

The term “K-value” refers to a number calculated from a dilute solution viscosity measurement of a polymer. The number denotes the degree of polymerization or molecular size and is a calculated parameter based on polymer concentration in water (c) and the corresponding measured relative viscosity [ηᵣₑₗ]:

$$\frac{log [ {η}_{rel} ]}{c} = \frac{75 {K}_{o}^{2}}{1 + 1.5 {K}_{o} c} + {K}_{o}$$

wherein the K-value equals 1000Kₒ. By rearranging the above equation, the Fikentscher equation provides a direct calculation of the K-value:

$$\frac{\sqrt{300 c log ( z ) + {( c + 1.5 c log ( z ) )}^{2}} + 1.5 c log ( z ) - c}{0.15 c + 0.003 {c}^{2}}$$

wherein z is the viscosity of the polymer at concentration c. For linear polymers, increasing polymer molecular weight increases its K-value. By way of comparison, the relationship between molecular weight, intrinsic viscosity ([|η|]), and K-value for linear PVP is provided in the chapter “N-vinyl amide polymers,” by Eugene S. Barabas, volume 17 of Encyclopedia of Polymer Science and Engineering, pages 198-257, 1989 (the contents of which are incorporated herein their entirety by reference).

The symbol of a “bond to the middle of group” means that the bond can be attached to either atom at the group, meaning the structure is referring to a mixture of isomers. For example, the structure below:

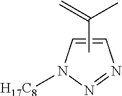

refers to both the 1,4- and 1,5-octyl methyl vinyl 1,2,3-triazole isomers.

The term “personal care composition” refers to a composition intended for use on or in the human body and may be an oral care composition, a hair care composition, a hair styling composition, a face care composition, a lip care composition, an eye care composition, a foot care composition, a nail care composition, a sun care composition, a deodorant composition, an antiperspirant composition, a cosmetic composition (including color cosmetics), a skin cleaning composition, an insect repellant composition, a shaving composition, a toothpaste, a mouthwash, a tooth whitener, a tooth stain remover, and/or a hygiene composition. Among their many uses, hair care and hair styling compositions find application in enhancing hair shine, cleansing hair, conditioning hair, repairing split ends, enhancing hair manageability, modulating hair stylability, protecting hair from thermal damage, imparting humidity resistance to hair and hair styles, promoting hair style durability, changing the hair color, straightening and/or relaxing hair, and/or providing protection from UV-A and/or UV-B radiation. Other personal care compositions, such as those for skin care and sun care compositions, are useful for protecting from UV-A and/or UV-B radiation, imparting water resistance or water proofness, moisturizing skin, decreasing and/or minimizing the appearance of wrinkles, firming skin, decreasing or minimizing the appearance of skin blemishes (such as lentigo, skin discolorations, pimples, or acne), changing skin color (such as color cosmetics for the face, cheeks, eyelids, or eye lashes). Oral care compositions according to the invention may be used as denture adhesives, toothpastes, mouthwashes, tooth whiteners, and/or stain removers. Personal care compositions also are used for delivering an active (such as to the skin, hair, or oral cavity).

The Examples provided later describe just a few of the many hair styling products that can be formulated with the branched polymers of the invention. Hair shampoos and hair conditions also may be created with the branched polymers. The TRESemmé 24 Hour Body Shampoo and TRESemmé Moisture Rich, Vitamin E Conditioner For Dry or Damaged Hair are two commercial products offered by The Alberto-Culver Company containing PVP. Branched PVPs of the invention may be used in these or other shampoos, conditioners or other hair care/hair styling products by one skilled in the art, taking into consideration the properties of the branched polymer(s). Included among the products that can be formulated are 2-in-1 shampoo-conditioners, and 3-in-1 shampoo-conditioner-body wash.

The term “pharmaceutical/nutritional composition” refers to any composition that comprises one or more chemical agents that elicit a response from a mammal (such as man, horse, dog, cat) when administered by topical, oral, sublingual, intravenous, subcutaneous, anal, or vaginal routes. These chemical agents may be natural or synthetic, of any purity, and may be the active agent itself or a form subsequently converted to the active agent. Non-limiting examples include prescriptive medications, over the counter (OTC) medications, nutritional supplements, dietary supplements, and vitamins, and may have the product format of a tablet, caplet, softgel, chewable, multi-particulate capsule, powder, cream, lotion, ointment, paste, solution, dispersion, emulsion, gel, shampoo, rinse, dip, wipe, or injectable.

The term “performance chemicals composition” refers to compositions that are not personal care compositions nor pharmaceutical/nutritional compositions. Performance chemicals compositions serve a broad variety of applications, non-limiting examples of which include: adhesives, agricultural, biocides, veterinary, coatings, electronics, household-industrial-institutional (HI&I), inks, membranes, metal fluids, oilfield, paper, paints, plastics, printing, plasters, textiles, fuels, lubricants, home care, and wood care compositions.

### Polymers

Provided are branched polymers that are synthesized from at least one reactive monomer (A1) and/or (A2) and at least one branching agent (B1) and/or (B2). Reactive monomer (A1) comprises at least one vinyl functional group, and reactive monomer (A2) is a hybrid reactive monomer having (a) at least one vinyl functional group, and (b) at least one other reactive non-vinyl functional group. The branching agent (B1) comprises at least two N-vinyl formamide functional groups, and the branching agent (B2) is a hybrid branching agent having (a) at least one N-vinyl formamide functional group and (b) at least one vinyl functional group. While four families of branched polymers are summarized in Table 1, note is made that combinations of the reactive monomers (A1) and/or (A2) and branching agents (B1) and/or (B2) are within the scope of the invention.

TABLE 1
At least four polymer families contemplated by the invention

| polymer | reactive monomer | branching agent |
|---|---|---|
| family | (A1) and (A2) | (B1) and (B2) |
| 1 | (Al) a reactive monomer having a vinyl | (B1) a branching agent having at |
| | functional group | least two N-vinyl formamide |
| | | functional groups |
| 2 | (A1) a reactive monomer having a vinyl | (B2) a hybrid branching agent |
| | functional group | having (a) at least one N-vinyl |
| | | formamide functional group, and |
| | | (b) at least one vinyl functional |
| | | group |
| 3 | (A2) a hybrid reactive monomer having | (B1) a branching agent having at |
| | (a) at least one vinyl functional group and | least two N-vinyl formamide |
| | (b) at least one reactive, non-vinyl | functional groups |
| | functional group | |
| 4 | (A2) a hybrid reactive monomer having | (B2) a hybrid branching agent |
| | (a) at least one vinyl functional group and | having (a) at least one N-vinyl |
| | (b) at least one reactive, non-vinyl | formamide functional group, and |
| | functional group | (b) at least one vinyl functional |
| | | group |

In polymer families #1 and #2, the reactive monomer (A1) comprises at least one vinyl functional group. One skilled in the art recognizes that these reactive monomers may be selected from the group consisting of maleic anhydrides, vinyl amides, acrylates, styrenes, maleimides, maleates, fumarates, cinnamyls, vinyl imidazoles, vinyl pyridines, vinyl acetates, acrylamides, vinyl sulfones, vinyl carbonates, vinyl silanes, vinyl acrylamides, allyls, vinyl ethers, and mixtures thereof.

Specific examples of the abovementioned reactive monomer (A1) include, without limitation:

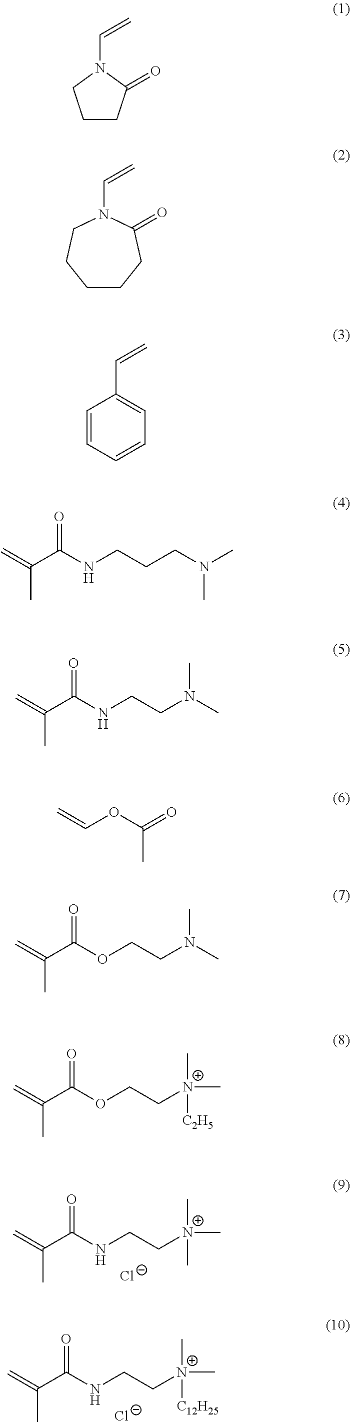

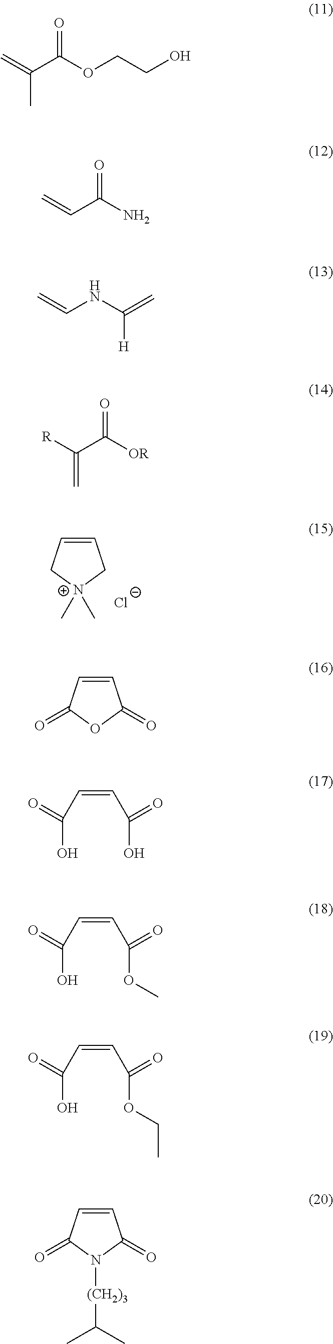

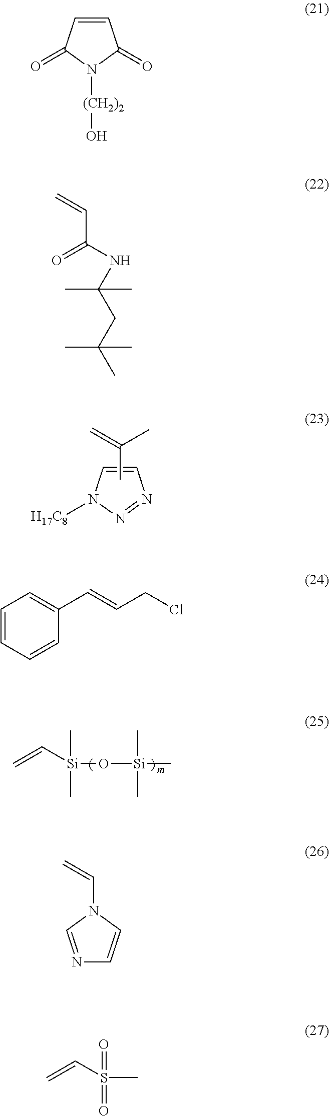

wherein each R is independently selected from the group consisting of hydrogen, and functionalized and unfunctionalized alkyl, cycloalkyl, alkenyl, and aryl groups, wherein any of the beforementioned groups may be with or without heteroatoms, and m and n are integers greater than or equal to 1.

Also included in polymer families #1 and #2 are branching agents (B1) or (B2). For polymer family #1 the branching agent (B1) comprises at least two N-vinyl formamide functional groups. This branching agent can be represented by the generic structure:

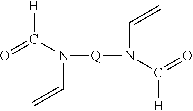

wherein the spacer arm Q is selected from the group consisting of functionalized and unfunctionalized alkylene of linear, branched, or cyclic structure and combinations thereof, wherein any of the beforementioned groups may be with or without oxygen atom(s).

Non-limiting examples of branching agent (B1) include the following:

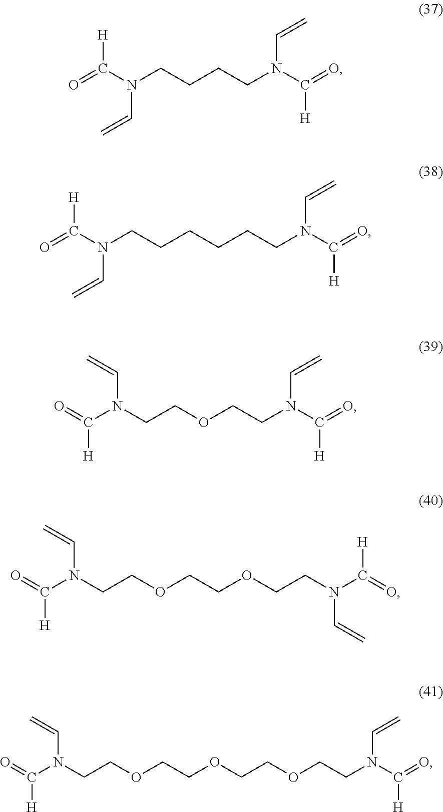

and combinations thereof. In various embodiments, the branching agent (B1) is selected from the group represented by structures (37), (40), and (41).

Examples of polymers belonging to family #1 include the branched homopolymers of N-vinyl-2-pyrrolidone:

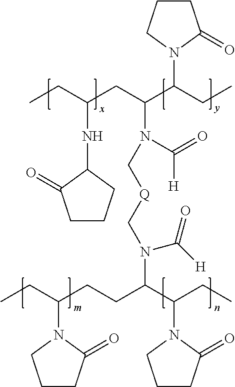

and N-vinyl-2-caprolactam:

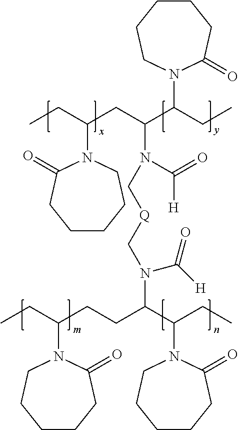

and the branched copolymer of N-vinyl-2-pyrrolidone and N-vinyl-2-caprolactam:

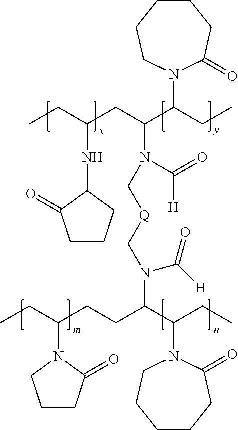

wherein Q retains the definition set forth above, and the subscripts m, n, x, and y are integers greater than 1 that denote the number of repeating units between the points of chain attachment via the branching agent.

Turning now to the polymer family #2, the branching agent is (B2), which is a hybrid branching agent having (a) at least one N-vinyl formamide functional group, and (b) at least one vinyl functional group. Examples of such are selected from the group consisting of:

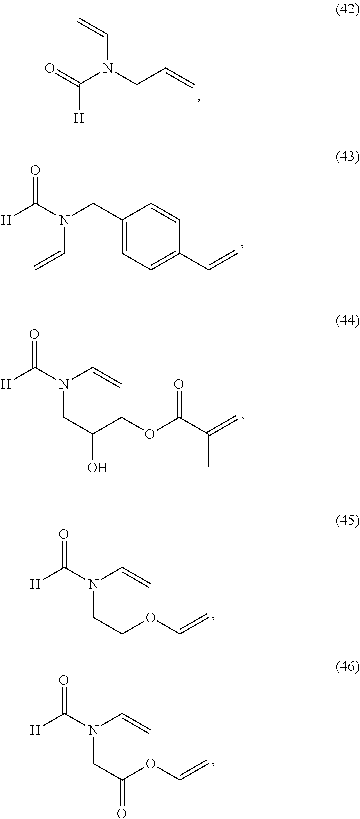

and combinations thereof.

More particularly, the branching agent (B2) for polymer family #2 is selected from the group represented by structures (42), (43), and (44).

Continuing the discussion of the invention's branched polymers, a different reactive monomer (A2) is provided in polymer families #3 and #4. For these families (A2) is a hybrid reactive monomer having (a) at least one vinyl functional group and (b) at least one reactive, non-vinyl functional group. Suitable reactive, non-vinyl functional groups include one or more of the following: epoxides, oxetanes, benzoxazines, oxazolines, and mixtures thereof. Examples of (A2) reactive monomers include the monomers represented by structures (1) through (36) that are modified to comprise at least one of these aforenamed reactive, non-vinyl functional groups. Just two of the many possibilities for the hybrid reactive monomer (A2) are:

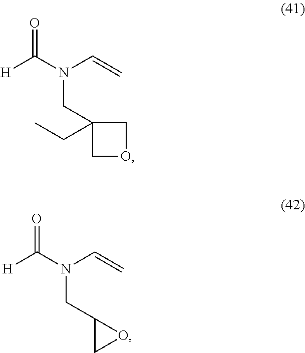

and combinations thereof. One knowledgeable in the art can easily identify other (A2) reactive monomers.

Polymer families #3 and #4 also require at least one branching agent, which are the (B1) branching agent (family #3) or the (B2) branching agent (family #4). Descriptions of these branching agents are exactly as provided in polymer families #1 and #2.

### Branched Polymer Syntheses

The polymers may be synthesized using at least one reactive monomer (A1) or (A2), and at least one branching agent (B1) or (B2), each as outlined above. Non-limiting structures (37) through (42) are presented in U.S. Pat. No. 5,300,606, which is incorporated herein its entirety by reference. The molecules also may be synthesized as described in PCT/US11/20208. Structures (41) is provided in U.S. Pat. No. 5,534,174 and a synthesis method also is given in PCT/US11/20208. Compounds represented by structures (42) through (46) are taught in international application WO 2009/099436.

Polymerization methods known to one skilled in the art may be employed to create the polymers described herein. These methods include, but are not limited to: free radical polymerization, emulsion polymerization, ionic chain polymerization, and precipitation polymerization. Free radical polymerization is a one polymerization method, which may be attractive when using water-dispersible and/or water-soluble reaction solvent(s), and is described in “Decomposition Rate of Organic Free Radical Polymerization” by K. W. Dixon (section II in Polymer Handbook, volume 1, 4th edition, Wiley-Interscience, 1999), which is incorporated by reference. Another description of the free-radical polymerization process is given in U.S. Pat. No. 2,882,262. Other polymerization methods, such as emulsion polymerization, suspension polymerization, gel polymerization, bead polymerization, and powder polymerization, also may be employed based on considerations such as final polymer form and ease of production.

The reactants, comprising at least one reactive monomer (A1) or (A2), and at least one branching agent (B1) or (B2) (e.g., from Table 1), may be charged together into a reactor and stirred at a temperature to facilitate the reaction, being limited only by the decomposition temperature of any reactant. The reaction can be performed with and without added solvent. The addition of an optional inert solvent may be beneficial when a high viscosity of the reacting system limits effective reactive processing (i.e., has a high viscosity). The reactants can be added to the reactor in one or multiple charges. The latter may be useful to help control the molecular weight, molecular weight distribution, and/or branching vs. crosslinked content. The Examples described later illustrate some of the possible addition schemes.

It is within the scope of this invention to employ any combination of reactive monomer (A1) and/or (A2), and branching agent (B1) and/or (B2). It may be advantageous to add the least reactive reactants first, and the more reactive ones later in the preparation. As necessary, additional reactive species can be attached to the polymer.

There is great flexibility in selecting the addition levels of the reactive monomers (A1) and/or (A2), which results in a wide range of properties for the described polymers. For example, when the polymer is a copolymer, then a first reactive monomer may comprise from about 1 mol % to about 99 mol % of the copolymer, and a second reactant may comprise from about 1 mol % to about 99 mol % of the total copolymer, providing balance for at least one branching agent (B1) and/or (B2). Alternatively, the same molar range applies to each polymerizable unit when the polymer is a terpolymer.

It is impossible, however, to define a priori a single concentration of the branching agent (B1) or (B2) in order to illustrate ranges that define branched polymers from 100% linear polymers and from fully crosslinked polymers. Although it is a convenient mental artifact, this boundary cannot be prescribed independent of the reaction system [i.e., reactants, branching agent, concentrations, solvent(s), chain transfer agent(s), initiator, reaction time], since these aspects when taken together determine the resulting polymer structure. Some of these aspects are illustrated in the Examples. Nonetheless, the amount of branching agent that distinguishes branched polymers from their fully crosslinked counterparts can be determined by one skilled in the art by measuring the polymer's solubility. In one embodiments, the branched polymers are those polymers synthesized with sufficient branching agent that are at least 10% (weight polymer/weight solvent+polymer) soluble in a same solvent that solubilizes the corresponding polymer of equal molecular weight made without the branching agent. In one aspect of this embodiment, the branched polymer is at least 25% soluble in the solvent, and most particularly the branched polymer's solubility is at least 50%. One may consider that the amount of branching agent (B1) and/or (B2) is less than about 10%, based on the total weight of reactant monomers (A1) and/or (A2). In other aspects the amount of branching agent (B1) and/or (B2) is less than 5%, and the addition level also may be less than 3%.

It may be beneficial or desirable to remove any amount of unreacted reactant and/or side product from the final reaction product using methods that are known in the art.

The reaction may be carried out for times ranging from 30 seconds to 48 hours or even more, and may depend upon factors that include (1) the reactivity of the reactants, (2) the number of reactive groups, since one or more of the reactants may have more than one reactive group, (3) steric hindrance surrounding any reactive site, (4) the reaction temperature employed, (5) the presence or absence of a solvent, and (6) the use or non-use of an initiator and/or catalyst. With the use of an optional reaction solvent or solvents, the solvent(s) may be removed after the reaction, e.g., at reduced pressure and/or elevated temperature, and then to add a different solvent conducive to the final formulation.

The invention allows for the polymerization of a wide range of molecular weights, so that the polymer scientist can create polymers that suit many different application needs. Typically, the weight-average molecular weight of the formed polymer ranges from about 200 Da to about 20,000,000 Da, and more particularly the weight-average molecular weight ranges from about 2,000 Da to about 10,000,000 Da. In certain aspects of the invention, the weight-average molecular weight ranges from about 5,000 Da to about 1,000,000 Da. As described later, the molecular weight of a polymerized product may be modulated by the addition of an optional chain transfer agent to the reaction vessel.

For solution reactions, temperatures may be conveniently controlled by judicious choice of solvents within an appropriate boiling range. A reaction system's temperature is only limited in as much as not to cause substantial decomposition of the reactants or solvent. In practice, a wide range in temperatures is possible. For example, temperatures can vary from about 20° C. to about 225° C., such as from 20° C. to about 100° C., and such as from 25° C. to about 80° C. Reaction times for solvent reaction range from several minutes to 48 hours or more. Higher reaction temperatures and highly reactive reactants will reduce time for conversion to the desired product(s). In some aspects of the invention, solvent reaction times may be between 15 minutes and 8 hours and may range between 15 minutes and 4 hours. In addition, azeotropic water removal (when possible) from the solvent will facilitate solvent reactions.

In some synthesis routes, an initiator is not needed to produce the disclosed polymers. Due to the broad nature of the invention, there may be times when an free radical addition polymerization initiator may be beneficial. The Examples presented later also illustrate these reaction schemes.

Compounds capable of initiating the free-radical addition polymerization include those materials known to function in the prescribed manner, and include the peroxo and azo classes of materials. Exemplary peroxo and azo compounds include, but are not limited to: acetyl peroxide; azo bis-(2-amidinopropane) dihydrochloride; azo bis-isobutyronitrile (AIBN); 2,2′-azo bis-(2-methylbutyronitrile); benzoyl peroxide; di-tert-amyl peroxide; di-tert-butyl diperphthalate; butyl peroctoate; tert-butyl dicumyl peroxide; tert-butyl hydroperoxide; tert-butyl perbenzoate; tert-butyl permaleate; tert-butyl perisobutylrate; tert-butyl peracetate; tert-butyl perpivalate; para-chlorobenzoyl peroxide; cumene hydroperoxide; diacetyl peroxide; dibenzoyl peroxide; dicumyl peroxide; didecanoyl peroxide; dilauroyl peroxide; diisopropyl peroxodicarbamate; dioctanoyl peroxide; lauroyl peroxide; octanoyl peroxide; succinyl peroxide; and bis-(ortho-toluoyl)peroxide. AIBN is one such initiator for a number of the compositions described herein.

Also suitable to initiate the free-radical polymerization are initiator mixtures or redox initiator systems, including: ascorbic acid/iron(II) sulfate/sodium peroxodisulfate, tert-butyl hydroperoxide/sodium disulfite, and tert-butyl hydroperoxide/sodium hydroxymethanesulfinate.

A chain transfer agent optionally may be used to control the polymer's molecular weight, molecular weight distribution, and/or branching character. As a skilled artisan can appreciate, typically, the chain transfer agent becomes part of the polymer.

The chain transfer agent may be of the kind that has a carbon-sulfur covalent bond. The carbon-sulfur covalent bond has usually absorption peak in a wave number region ranging from 500 cm⁻¹ to 800 cm⁻¹ in an infrared absorption spectrum. When the chain transfer agent is incorporated into the polymer, the absorption peak of the product may be changed in comparison to product made without a chain transfer agent.

Exemplary chain transfer agents include, but are not limited to, n-C3-15 alkylmercaptans such as n-propylmercaptan, n-butylmercaptan, n-amylmercaptan, n-hexylmercaptan, n-heptylmercaptan, n-octylmercaptan, n-nonylmercaptan, n-decylmercaptan, and n-dodecylmercaptan; branched alkylmercaptans such as isopropylmercaptan, isobutylmercaptan, s-butylmercaptan, tert-butylmercaptan, cyclohexylmercaptan, tert-hexadecylmercaptan, tert-laurylmercaptan, tert-nonylmercaptan, tert-octylmercaptan, and tert-tetradecylmercaptan; aromatic ring-containing mercaptans such as allylmercaptan, 3-phenylpropylmercaptan, phenylmercaptan, and mercaptotriphenylmethane. As a skilled artisan understands, the term -mercaptan and -thiol may be used interchangeably to mean C—SH group.

Typical examples of such chain transfer agents also include, but are not limited to, dodecanethiol, butanethiol, isooctyl-3-mercaptopropionate, 2-methyl-5-tert-butyl-thiophenol, carbon tetrachloride, carbon tetrabromide, and the like. Exemplary chain transfer agents include, but are not limited to, 2-mercaptoethanol, dodecanethiol, and carbon tetrabromide.

Based on total weight of the monomers to be polymerized, the chain transfer agent may generally be present in an amount from about 0.1% to about 7%, including from about 0.5% to about 6%, and from about 1.0% to about 5%, although it may be present in greater or lesser amounts.

A catalyst, such as dibutyltin dilaurate, used in the customary amounts, can be used to alter the reaction rate, e.g., speeding the reaction and/or favoring one product over side products.

### Description of Formulations Comprising the Branched Polymers

Polymers summarized herein may be used alone or in formulations with other ingredients in any number of arts. These compositions may take special advantage of the polymers' properties, including lower viscosity, enhanced processability, or improved sprayability compared to a corresponding polymer of equal molecular weight not having the branching agent (B1) and/or (B2). Broadly speaking, the invention enables the use of these polymers in the personal care arts, and in other areas, including adhesives, aerosols, agricultural compositions, beverages, cleaning compositions, coating compositions, dental compositions, detergents, drugs, encapsulations, foods, hair sprays, lithographic solutions, membrane formulations, oilfield formulations, pharmaceuticals, and pigment dispersions. Specific examples of personal care compositions in which the polymers find use are those compositions used on/for the hair, skin (including for protecting against aging, dryness, and sun damage), nails, and dental care.

Special classes of compositions according to the invention are those wherein the benefits of a higher molecular weight polymers are realized while exploiting the lower viscosity, enhanced processability, and/or improved sprayability, the invention's polymers. Several properties are known to improve with a polymer's molecular weight, including (without limitation): film strength, binding capability and binding strength, extension modulus, tensile strength, polymer processability (which may increase with polymer molecular weight and then decrease with additional increases in molecular weight due to insufficient viscosity/melt flow). The branching nature of the polymers described herein enable these compositions to benefit from the higher molecular weight but not the noted limitations.

Polymers of the invention may be used in a wide variety of compositions that serve the human condition, as well as in adhesives, agricultural, biocides, coatings, electronics, household-industrial-institutional (HI&I), inks, membranes, metal fluids, oilfield, paper, paints, plastics, printing, plasters, and wood-care compositions.

Compositions belonging to the personal care/cosmetic and pharmaceutical arts can find utility in altering, delivering an active, enhancing, improving, modifying the appearance, condition, color, health, style of the skin (including face, scalp, and lips), hair, nails, and oral cavity. Many examples and product forms of these compositions are known. These compositions can impart benefits that include, but are not limited to, hair style flexibility, hair style durability, humidity resistance for hair, color and/or color protection, moisturization, wrinkle reduction, protection from ultraviolet radiation, water proofness, water resistance, wear resistance, thermal protection, stabilization, adhesion, active ingredient delivery, anti-cavity and/or anti-gingivitis protection, stain removal, and/or stain prevention. As such, these compositions are sometimes categorized in the following areas: skin care, hair care (both styling and non-styling), sun care, cosmetics (including color cosmetics), antiperspirants, deodorants, oral hygiene, and men's and women's personal hygiene/grooming. In some cases these benefits and care areas overlap with another.

Skin care compositions include those materials used on the body, face, hands, lips, and/or scalp, and are beneficial for many reasons, such as firming, anti-cellulite, moisturizing, nourishing, cleaning, reducing or eliminating the appearance of wrinkles or lentigo, toning, and/or purifying. They also can be used to sanitize.

Today's savvy consumer can identify many of the compositions that serve the sun care area, for example after-fun, children's, beach, self-tan, sports (i.e., being sweatproof, waterproof, resistant to running, or having added UV absorbers and/or antioxidants), sensitive skin products (i.e., having low irritation to the eyes and/or skin, and/or being free of fragrances and/or dyes), daily wear, leave-on hair creams, lotions, styling products, and hair sprays. Typically, sun care products also comprise one or more UV actives, which are those organic and inorganic materials that scatter, absorb, and/or reflect radiation having a wavelength from about 100 nm to about 400 nm. In one aspect, the sun care product protects against UV-A and/or UV-B radiation. UV-A radiation, from about 320 nm to about 400 nm, has the longest wavelength within the UV spectrum, and consequently is the least energetic. While UV-A rays can induce skin tanning, they are liable to induce adverse changes as well, especially in the case of sensitive skin or of skin which is continually exposed to solar radiation. In particular UV-A rays cause a loss of skin elasticity and the appearance of wrinkles, leading to premature skin aging. UV-B rays have shorter wavelengths, from about 290 nm to about 320 nm, and their higher energy can cause erythema and skin burns which may be harmful. Alternatively, sun care products may omit UV actives, and may be regarded as a tanning oil or a tan promoter. Some sun care compositions may promote soothe skin after sun exposure, and/or be formulated for application to the lips, hair, or the area around the eyes. Self-tan compositions, which are products that color skin without requiring full sun exposure, also fit under the sun care umbrella. The many different sun care product formats include may assume a consistency ranging from liquid to semiliquid forms (e.g., milks, creams), to thicker forms like gels, creams, pastes, and even solid- and wax-like forms. Sun care products also may take the form of an aerosol, spray, mist, roll-on, or wipe.

Hair care compositions include shampoos, leave-on and rinse-out conditioners used for conditioning, moisturizing, repairing, hair colors, hair relaxers, and deep conditioners and treatments such as hot oils and waxes, 2-in-1 shampoo/conditioner combination products, 3-in-1 shampoo/conditioner/styling agent. The many types of hair care products can be delivered in an array of formats, including aerosol sprays, pump sprays, gel sprays, mousses, gels, waxes, creams, lotions, waxes, glues, pomades, spritzes, putties, lacquers, de-frizzing serums, perms, relaxants and colorants.

Color cosmetic compositions include facial make-up, eye makeup, mascaras, lip and nail products. Facial make-up compositions include foundation (liquid, solid, and semi-solid)—skin tinted creams, liquid, sticks, mousses used as a base under make-up, rouge, face powder, blusher, highlighters, face bronzers, concealers, and 2-way cake products.

Personal care/cosmetics also include eye make-up, mascaras, eyeliners, eye shadows, eyebrow pencils and eye pencils. Lip products include lipsticks, lip pencils, lip gloss, transparent bases and tinted lip moisturizers as well as multi-function color sticks that can also be used for cheeks and eyes. Nail products includes nail varnishes/enamels, nail varnish removers, treatments, home-manicure products such as cuticle softeners and nail strengtheners.

In addition to the skin, hair, and sun care compositions summarized above, the polymers related herein also find application in oral care compositions. Non-limiting examples or oral care compositions include toothpastes (including toothpaste gels), mouthwashes, mouth rinses, chewing gums, denture adhesives, whiteners, stain removers, anesthetics, and dental floss and related products. Furthermore, the oral care compositions may include one or more optional active ingredient(s), such as (but not limited to) pain relievers, anti-inflammatory agents, stimulants, depressants, diet aides, smoking cessation aides, vitamins, minerals, tooth whitening agents, anti-plaque agents, warming or tingling agents, throat-soothing agents, spices, herbs, herbal extracts, caffeine, and/or nicotine. These compositions may take any product format, such as pastes, gels, creams, solutions, dispersions, rinses, flosses, aerosols, powders, strips, films, pads, and lozenges.

In one embodiment, the oral care composition comprises branched poly(N-vinyl-2-pyrrolidone) (PVP) that may be combined with other polymers having linear, branched, or fully crosslinked structures. These compositions may be used to reduce and/or prevent the adherence of oral bacterial to the tooth enamel, such as described in U.S. Pat. No. 5,538,718, the contents of which are hereby incorporated herein their entirety by reference.

Other embodiments of the invention contemplate oral care compositions comprising a non-homopolymer having a linear, branched, random, graft, and/or block structure. Such non-homopolymers include those polymerized in part from N-vinyl-2-pyrrolidone and a C1-C19 alkyl carboxylic acid C2-C12 ester monomer, such as those taught in U.S. Pat. No. 6,682,722 (the contents of which are hereby incorporated in their entirety by reference). In other embodiments, the oral care composition may comprise copolymers of maleic anhydride and alkyl vinyl ether(s), including, but not limited to poly(maleic anhydride-co-methyl vinyl ether) polymers that are sold into commercial trade under the trade name Gantrez® (ISP), and those disclosed in U.S. Pat. Nos. 5,003,014; 5,449,715; and 6,184,325. The contents of the '014, '715, and '325 patents are hereby incorporated herein their entirety by reference.

Also contemplated are oral care compositions comprising the branched polymers described herein with hydrogen peroxide. In one aspect of this embodiment, the branched polymer is branched PVP. For example the combination of the branched PVP and hydrogen peroxide can be a substantially anhydrous complexes, and can take the form of free-flowing, fine white powders such that are useful in reducing the microbial content of surfaces. A description of these complexes is described in international application WO 91/07184, the contents of which are hereby incorporated in their entirety by reference.

Also contemplated are oral care compositions described in the following patents and patent applications, the contents of each are hereby incorporated herein their entirety by reference: WO 2011/068514, WO 2011/053877, US 2010/0275394, US 2011/0076090, US 2008/091935, US 2008/0181716, US 2008/0014224, WO 2007/066837, US 2008/0292669, US 2007/0071696, US 2007/0154863, US 2008/0317797, US 2005/0249678, US 2007/0178055, US 2007/0189983, WO 2005/041910, U.S. Pat. No. 7,785,572, WO 1998/005749, WO 1997/022651, and U.S. Pat. No. 5,310,563.

Similarly, the invention also contemplates compositions comprising the branched polymer(s) described herein with iodine. Consider one embodiment, wherein the branched polymer is branched PVP. In these embodiments the branched polymer/iodine composition may find applications in personal care compositions and methods, as well as non-personal care compositions and methods. The compositions may be used in treating skin conditions, such as periodontal disease, as well as dermatitis, wounds, bacterial infections, burns, rashes, herpes, and other disease states. These complexed compositions may be staining, substantially non-staining, or essentially non-staining Examples of branched polymer/iodine compositions include those taught in the following patents and patent applications, the contents of which are hereby incorporated in their entirety by reference: U.S. Pat. No. 4,017,407, U.S. Pat. No. 4,320,114, U.S. Pat. No. 4,592,488, U.S. Pat. No. 4,560,553, U.S. Pat. No. 5,242,985, U.S. Pat. No. 7,927,498, US 2006/0198814, US 2008/0206164, US 2010/0076546, US 2010/0247631, GB 1,533,406, EP 27613, EP 507,771, EP 793,418, EP 1,449,520, and WO 2011/035288.

Men's and women's grooming products includes shaving products and toiletries, which may find use in preparing the skin and/or hair for dry or wet shaving. In addition, these compositions may help to moisturize, cool, and/or soothe skin. A variety of product forms are known, a few of which are foams, gels, creams, sticks, oils, solutions, tonics, balms, aerosols, mists, sprays, and wipes.

The polymer can also be used in other personal care/cosmetic applications, such as an absorbent material in appropriate applications such as diapers, incontinence products, feminine products, and other related products.

The polymers described herein also find application in bath and shower compositions, such as foams, gels, salts, oils, balls, liquids, powders and pearls. Also included are bar soaps, body washes, shower gels, cleansers, gels, oils, foams, scrubs and creams. As a natural extension of this category, these compositions also include liquid soaps and hand sanitizers used for cleaning hands.

Hair care formulations are just one example of the invention's formulations. These compositions can take advantage of the properties imparted by the branched polymers, most typically when used in combination with other formulation ingredients. Examples of suitable hair care formulas may be used to style (i.e., sculpt, mold, curl, scrunch, add volume, define, shape), enhance, rejuvenate, fix, maintain, and/or hold hair styling and appearance. Representative products that are hair care compositions include, without limit, hairsprays, hair styling products, gels, mousses, creams, mists, conditioners, tonics, waxes, pomades, serums, setting lotions, and volumizers. They may or may not contain added materials to enable atomization/spraying.

Branched polymers according to the invention also find application in the pharmaceutical/nutritional compositions, wherein the branched polymers may be used in coatings, binders, solubilizers, stabilizers, dispersing aides, film formers, and/or disintegrants. For example, it may be beneficial to produce a branched PVP for pharmaceutical/nutritional compositions to take advantage of the enhancing processing properties noted earlier. Additional insight into how these branched polymers find application in this art area include the following publications by International Specialty Products: Health and nutrition product guide—Performance enhancing products (August 2008), Plasdone® povidones product overview (April 2010), Plasdone® K-12 and K-17 providones—Solubilizers for liquid softgel fill formulations (September 2010), Plasdone® K-29/32 povidone—High efficiency binder for wet granulation (April 2010), Plasdone® S-630 copovidone—Product Overview (April 2010), Polyplasdone® Ultra and Ultra-10 crospovidones—Product overview (Sep 2010), Polyplasdone® superdisintegrants—Product overview (July 2010), Polyplasdone® crospovidone—Superdisintegrants for orally disintegrating and chewable tablets (July 2010), Polyplasdone® crospovidone—Nonionic superdisintegrant for improved dissolution of cationic drugs (July 2009), Polyplasdone® crospovidone—The solution for poorly soluble drugs (July 2009), Polyplasdone® crospovidone—Novel pelletization aid for extrusion spheronization (July 2010), PVP-Iodine povidone iodine antiseptic agent (March 2004), and Pharmaceutical technical bulletin—PVP-Iodine for prophylaxis and treatment of bovine mastitis (December 2003). Each publication is hereby incorporated in its entirety be reference.

An array of additional compositions, methods, and uses are contemplated by the invention. In some, but not all cases, these compositions resemble compositions having linear, lightly-crosslinked, or fully crosslinked PVP. Disclosure of these compositions may be found in the following brochures by International Specialty Products, each of which is hereby incorporated in its entirety by reference: Plasdone® K-29/32, Advanced non-oxidative, non-abrasive teeth whitening in toothpasts, mouthwashes, and oral rinses (2010), Polymers for oral care, product and applications guide (2002), A formulation guide for excellent hair styling gels and lotions (April 2003), PVP (polyvinylpyrrolidone) (no date provided), and Textile chemicals, solutions for the most challenging product environment (no date provided).

### Optional: Additional Formulation Ingredients and Adjuvants

Due to the requirements of end performance, in many cases compositions of this invention will be used together with other additives to further enhance the properties of the finished product. Such ingredients may be incorporated without altering the scope of the current invention, and may be included in order to produce the necessary products. In one embodiment, the ingredients may be used in personal care compositions, such as skin care, hair care, oral care, and sun care.

It may be desirable to include one or more ingredients described in the prior art disclosure IPCOM000186541D, IPCOM000128968D, and IPCOM000109682D on www.ip.com, the contents of this disclosure are incorporated herein their entirety by reference.

Further reference to formulary co-ingredients and product forms include the disclosures in US 2010/0183532, paragraphs [0096]-[0162] and WO 2010/105050, paragraphs [0053]-[0069], the contents of which are hereby incorporated by reference.

For some embodiments, it may be preferred to add one or more preservatives and/or antimicrobial agents, such as, but not limited to, benzoic acid, sorbic acid, dehydroacetic acid, diazolidinyl urea, imidazolidinyl urea, salicylic acid, piroctone olamine, DMDM hydantoin, IPBC, triclosan, bronopol, formaldehyde, isothiazolinones, nitrates/nitrites, parabens, phenoxyethanol, potassium sorbate, sodium benzoate, sulphites, and sulphur dioxide. Combinations of preservatives may be used.

In other embodiments it may be desirable to incorporate preservative boosters/solvents, select examples of which include caprylyl glycol, hexylene glycol, pentylene glycol, ethylhexylglycerin, caprylhydroxamic acid, caprylohydroxamic acid, and glyceryl caprylate.

Humectants, which include glycerin, butylene glycol, propylene glycol, sorbitol, mannitol, and xylitol may be added.

Polysaccharides, such as gum Arabic, may be included as well.

When the polymers embraced by the present invention are formulated into oral care products, optional ingredients include those materials known in such products. These materials may include: carriers, dentifrices, antibacterial agents, anticalculus agents, thickeners, gelling agents, surfactants, flavors, warming agents, tooth bleaching agents and whiteners, abrasives, colors, emollients, emulsifiers, preservatives, solvents, binders, and humectants.

The choice of a carrier to be used is basically determined by the way the composition is to be introduced into the oral cavity. Carrier materials for toothpaste, tooth gel or the like include abrasive materials, sudsing agents, binders, humectants, flavoring and sweetening agents, as disclosed in e.g., U.S. Pat. No. 3,988,433. Carrier materials for biphasic dentifrice formulations are disclosed in U.S. Pat. Nos. 5,213,790; 5,145,666; 5,281,410; 4,849,213; and 4,528,180. Mouthwash, rinse or mouth spray carrier materials typically include water, flavoring and sweetening agents, etc., as disclosed in, e.g., U.S. Pat. No. 3,988,433. Lozenge carrier materials typically include a candy base; chewing gum carrier materials include a gum base, flavoring and sweetening agents, as in, e.g., U.S. Pat. No. 4,083,955. Sachet carrier materials typically include a sachet bag, flavoring and sweetening agents. For subgingival gels used for delivery of actives into the periodontal pockets or around the periodontal pockets, a “subgingival gel carrier” is chosen as disclosed in, e.g., U.S. Pat. Nos. 5,198,220 and 5,242,910. Carriers suitable for the preparation of compositions of the present invention are well known in the art. Their selection will depend on secondary considerations like taste, cost, and shelf stability, as well as other considerations.

Compositions of the invention also may comprise one or more of a dental abrasive (from about 6% to about 70%). Dental abrasives useful in the compositions of the subject invention include many different materials. The material selected must be one which is compatible within the composition of interest and does not excessively abrade dentin. Suitable abrasives include, for example, silicas including gels and precipitates, insoluble sodium polymetaphosphate, hydrated alumina, calcium carbonate, dicalcium orthophosphate dihydrate, calcium pyrophosphate, tricalcium phosphate, calcium polymetaphosphate, and resinous abrasive materials such as particulate condensation products of urea and formaldehyde.

Another class of abrasives for use in the present compositions is the particulate thermo-setting polymerized resins as described in U.S. Pat. No. 3,070,510. Suitable resins include, for example, melamines, phenolics, ureas, melamine-ureas, melamine-formaldehydes, urea-formaldehyde, melamine-urea-formaldehydes, cross-linked epoxides, and cross-linked polyesters.

Silica dental abrasives of various types may be employed because of their unique benefits of exceptional dental cleaning and polishing performance without unduly abrading tooth enamel or dentine. The silica abrasive polishing materials herein, as well as other abrasives, generally have an average particle size ranging between about 0.1 to about 30 microns, and particularly from about 5 to about 15 microns. The abrasive can be precipitated silica or silica gels such as the silica xerogels described in U.S. Pat. No. 3,538,230, and U.S. Pat. No. 3,862,307. Examples include the silica xerogels marketed under the trade name “Syloid” by the W.R. Grace & Company, Davison Chemical Division and precipitated silica materials such as those marketed by the J.M. Huber Corporation under the trade name, Zeodent®, particularly the silicas carrying the designation Zeodent® 119, Zeodent® 118, Zeodent® 109 and Zeodent® 129. The types of silica dental abrasives useful in the toothpastes of the present invention are described in more detail in U.S. Pat. Nos. 4,340,583; 5,603,920; 5,589,160; 5,658,553; 5,651,958; and 6,740,311.

Mixtures of abrasives can be used such as mixtures of the various grades of Zeodent® silica abrasives listed above. The total amount of abrasive in oral care compositions of the subject invention typically range from about 6% to about 70% by weight; toothpastes may contain from about 10% to about 50% of abrasives, by weight of the composition. Dental solution, mouth spray, mouthwash and non-abrasive gel compositions of the subject invention typically contain little or no abrasive.

The present invention may optionally include antimicrobial agents. Included among such agents are water insoluble non-cationic antimicrobial agents such as halogenated diphenyl ethers, phenolic compounds including phenol and its homologs, mono and poly-alkyl and aromatic halophenols, resorcinol and its derivatives, bisphenolic compounds and halogenated salicylanilides, benzoic esters, and halogenated carbanilides. The water soluble antimicrobials include quaternary ammonium salts and bis-biquanide salts, and triclosan monophosphate. The quaternary ammonium agents include those in which one or two of the substitutes on the quaternary nitrogen has a carbon chain length (typically alkyl group) from about 8 to about 20, typically from about 10 to about 18 carbon atoms while the remaining substitutes (typically alkyl or benzyl group) have a lower number of carbon atoms, such as from about 1 to about 7 carbon atoms, typically methyl or ethyl groups. Dodecyl trimethyl ammonium bromide, tetradecylpyridinium chloride, domiphen bromide, N-tetradecyl-4-ethyl pyridinium chloride, dodecyl dimethyl(2-phenoxyethyl)ammonium bromide, benzyl dimethylstearyl ammonium chloride, cetyl pyridinium chloride, quaternized 5-amino-1,3-bis(2-ethyl-hexyl)-5-methyl hexahydropyrimidine, benzalkonium chloride, benzethonium chloride and methyl benzethonium chloride are exemplary of typical quaternary ammonium antibacterial agents. Other compounds are bis[4-(R-amino)-1-pyridinium]alkanes as disclosed in U.S. Pat. No. 4,206,215. Other antimicrobials such as copper salts, zinc salts and stannous salts may also be included. Also useful are enzymes, including endoglycosidase, papain, dextranase, mutanase, and mixtures thereof. Such agents are disclosed in U.S. Pat. Nos. 2,946,725 and 4,051,234. The antimicrobial agents may comprise zinc salts, stannous salts, cetyl pyridinium chloride, chlorhexidine, triclosan, triclosan monophosphate, and flavor oils such as thymol. Triclosan and other agents of this type are disclosed in U.S. Pat. Nos. 5,015,466 and 4,894,220. These agents provide anti-plaque benefits and are typically present at levels of from about 0.01% to about 5.0%, by weight of the composition.

The present compositions may optionally include an additional anticalculus agent, such as a pyrophosphate salt as a source of pyrophosphate ion. The pyrophosphate salts useful in the present compositions include the dialkali metal pyrophosphate salts, tetraalkali metal pyrophosphate salts, and mixtures thereof. Disodium dihydrogen pyrophosphate (Na₂H₂P₂O₇), tetrasodium pyrophosphate (Na₄P₂O₇), and tetrapotassium pyrophosphate (K₄P₂O₇) in their unhydrated as well as hydrated forms may find utility. In compositions of the present invention, the pyrophosphate salt may be present in one of three ways: predominately dissolved, predominately undissolved, or a mixture of dissolved and undissolved pyrophosphate.

Compositions comprising predominately dissolved pyrophosphate refer to compositions where at least one pyrophosphate ion source is in an amount sufficient to provide at least about 1.0% free pyrophosphate ions. The amount of free pyrophosphate ions may be from about 1% to about 15%, from about 1.5% to about 10% in one embodiment, and from about 2% to about 6% in another embodiment. Free pyrophosphate ions may be present in a variety of protonated states depending on the pH of the composition.

Compositions comprising predominately undissolved pyrophosphate refer to compositions containing no more than about 20% of the total pyrophosphate salt dissolved in the composition, particularly less than about 10% of the total pyrophosphate dissolved in the composition. Tetrasodium pyrophosphate salt may be one such pyrophosphate salt in these compositions. Tetrasodium pyrophosphate may be the anhydrous salt form or the decahydrate form, or any other species stable in solid form in the oral care compositions. The salt is in its solid particle form, which may be its crystalline and/or amorphous state, with the particle size of the salt being small enough to be aesthetically acceptable and readily soluble during use. The amount of pyrophosphate salt useful in making these compositions is any tartar control effective amount, generally from about 1.5% to about 15%, particularly from about 2% to about 10%, and most particularly from about 3% to about 8%, by weight of the oral care composition.

Compositions may also comprise a mixture of dissolved and undissolved pyrophosphate salts. Any of the above mentioned pyrophosphate salts may be used.

The pyrophosphate salts are described in more detail in Kirk-Othmer Encyclopedia of Chemical Technology, third edition, volume 17, Wiley-Interscience Publishers (1982).

Thickening or gelling agents used in the formulation of the dentifrice include nonionic polyoxyethylene polyoxypropylene block copolymers. Illustrative of polyoxyethylene polyoxypropylene block copolymers useful in the practice of the present invention include block copolymers having the formula HO(C₂H₄O)_{b}(C₃H₆O₆)ₐ(C₂H₄O)_{b}H wherein a is an integer such that the hydrophobic base represented by (C₃H₆O₆) has a molecular weight of about 2,750 Da to 4000 Da, b is an integer such that the hydrophilic portion (moiety) represented by (C₂H₄O) constitutes about 70% to 80% by weight of the copolymer. Block copolymers of this composition are available commercially under the trademark Pluronic® F type.

Pluronic® F127 has a molecular weight of 4,000 Da and contains 70% of the hydrophilic polyoxyethylene moiety.

Also suitable as a thickening agent is lightly- to moderately-crosslinked PVP, described in co-pending U.S. provisional patent application U.S. 61/325,673.

The thickening agents may be present in an amount within the range of about 15% to about 50% by weight, and about 25% to about 45% by weight for use in the compositions of the present invention.

Surfactants may also be included in the oral care compositions, where they may serve in solubilizing, dispersing, emulsifying agents and agents that reduce the surface tension of the teeth in order to increase the contact between the tooth and the peroxide. The present compositions may also comprise surfactants, also commonly referred to as sudsing agents. Suitable surfactants are those which are reasonably stable and foam throughout a wide pH range. The surfactant may be anionic, nonionic, amphoteric, zwitterionic, cationic, or mixtures thereof.

Anionic surfactants useful herein include the water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms. Sodium lauryl sulfate (SLS) and sodium coconut monoglyceride sulfonates are examples of anionic surfactants of this type. Other suitable anionic surfactants are sarcosinates, such as sodium lauroyl sarcosinate, taurates, sodium lauryl sulfoacetate, sodium lauroyl isethionate, sodium laureth carboxylate, and sodium dodecyl benzenesulfonate. Mixtures of anionic surfactants can also be employed. Many suitable anionic surfactants are disclosed in U.S. Pat. No. 3,959,458. The present composition typically comprises an anionic surfactant at a level of from about 0.025% to about 9%, from about 0.05% to about 5% in some embodiments, and from about 0.1% to about 1% in other embodiments.

Another suitable surfactant is one selected from the group consisting of sarcosinate surfactants, isethionate surfactants and taurate surfactants. Useful are alkali metal or ammonium salts of these surfactants such as the sodium and potassium salts of the following: lauroyl sarcosinate, myristoyl sarcosinate, palmitoyl sarcosinate, stearoyl sarcosinate and oleoyl sarcosinate. The sarcosinate surfactant may be present in the compositions of the present invention from about 0.1% to about 2.5%, particularly from about 0.5% to about 2.0% by weight of the total composition.

Cationic surfactants useful in the present invention include derivatives of aliphatic quaternary ammonium compounds having one long alkyl chain containing from about 8 to 18 carbon atoms such as lauryl trimethylammonium chloride; cetyl pyridinium chloride; cetyl trimethylammonium bromide; di-isobutylphenoxyethyl-dimethylbenzylammonium chloride; coconut alkyltrimethylammonium nitrite; cetyl pyridinium fluoride; etc. Example compounds are the quaternary ammonium fluorides described in U.S. Pat. No. 3,535,421, where the quaternary ammonium fluorides have detergent properties. Certain cationic surfactants can also act as germicides in the compositions disclosed herein. Cationic surfactants such as chlorhexidine, although suitable for use in the current invention, may stain the oral cavity's hard tissues. Persons skilled in the art are aware of this possibility and should incorporate cationic surfactants with this limitation in mind.

Nonionic surfactants that can be used in the compositions of the present invention include compounds produced by the condensation of alkylene oxide groups (hydrophilic in nature) with an organic hydrophobic compound which may be aliphatic or alkylaromatic in nature. Examples of suitable nonionic surfactants include poloxamers (sold under the trade name Pluronic® by BASF Corp.), polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials.

Zwitterionic synthetic surfactants useful in the present invention include derivatives of aliphatic quaternary ammonium, phosphonium, and sulfonium compounds, in which the aliphatic radicals can be straight chain or branched, and wherein one of the aliphatic substituents contains from about 8 to 18 carbon atoms and one contains an anionic water-solubilizing group, e.g., carboxy, sulfonate, sulfate, phosphate or phosphonate.

Suitable betaine surfactants are disclosed in U.S. Pat. No. 5,180,577. Typical alkyl dimethyl betaines include decyl betaine or 2-(N-decyl-N,N-dimethylammonio)acetate, coco betaine or 2-(N-coc-N,N-dimethyl ammonio)acetate, myristyl betaine, palmityl betaine, lauryl betaine, cetyl betaine, cetyl betaine, stearyl betaine, etc. The amidobetaines are exemplified by cocoamidoethyl betaine, cocoamidopropyl betaine, lauramidopropyl betaine and the like. The betaines of choice include cocoamidopropyl betaines, such as lauramidopropyl betaine. Other surfactants such as fluorinated surfactants and surface tension reducing materials may also be incorporated within the compositions.

Traditional flavor components include methyl salicylate, ethyl salicylate, methyl cinnamate, ethyl cinnamate, butyl cinnamate, ethyl butyrate, ethyl acetate, methyl anthranilate, iso-amyl acetate, iso-armyl butyrate, allyl caproate, eugenol, eucalyptol, thymol, cinnamic alcohol, cinnamic aldehyde, octanol, octanal, decanol, decanal, phenylethyl alcohol, benzyl alcohol, benzaldehyde, α-terpineol, linalool, limonene, citral, vanillin, ethyl vanillin, propenyl guaethol, maltol, ethyl maltol, heliotropin, anethole, dihydroanethole, carvone, oxanone, menthone, β-damascenone, ionone, gamma decalactone, gamma nonalactone, gamma undecalactone, 4-hydroxy-2,5-dimethyl-3(2H)-furanone and mixtures thereof. Generally suitable flavoring agents are those containing structural features and functional groups that are less prone to oxidation by peroxide. These include derivatives of flavor chemicals that are saturated or contain stable aromatic rings or ester groups. Also suitable are flavor chemicals that may undergo some oxidation or degradation without resulting in a significant change in the flavor character or profile. The flavor chemicals including menthol may be provided as single or purified chemicals rather than supplied in the composition by addition of natural oils or extracts such as peppermint, spearmint or wintergreen oils as these sources may contain other components that are relatively unstable and may degrade in the presence of peroxide. Such degradation may significantly alter the desired flavor profile and result in a less acceptable product from an organoleptic standpoint. Flavoring agents are generally used in the compositions at levels of from about 0.001% to about 5%, by weight of the composition.

The flavor system will typically include a sweetening agent. Suitable sweeteners include those well known in the art, including both natural and artificial sweeteners. Some suitable water-soluble sweeteners include monosaccharides, disaccharides and polysaccharides such as xylose, ribose, glucose (dextrose), mannose, galactose, fructose (levulose), sucrose (sugar), maltose, invert sugar (a mixture of fructose and glucose derived from sucrose), partially hydrolyzed starch, corn syrup solids, dihydrochalcones, monellin, steviosides, and glycyrrhizin. Suitable water-soluble artificial sweeteners include soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (acesulfame-K), the free acid form of saccharin, and the like. Other suitable sweeteners include Dipeptide based sweeteners, such as L-aspartic acid derived sweeteners, such as L-aspartyl-L-phenylalanine methyl ester (aspartame) and materials described in U.S. Pat. No. 3,492,131, L-alpha-aspartyl-N-(2,2,4,4-tetramethyl-3-thietanyl)-D-alaninamide hydrate, methyl esters of L-aspartyl-L-phenylglycerin and L-aspartyl-L-2,5,dihydrophenyl-glycine, L-aspartyl-2,5-dihydro-L-phenylalanine, L-aspartyl-L-(1-cyclohexyen)-alanine, and the like. Water-soluble sweeteners derived from naturally occurring water-soluble sweeteners, such as a chlorinated derivative of ordinary sugar (sucrose), known, for example, under the product description of sucralose as well as protein based sweeteners such as thaumatoccous danielli (Thaumatin I and II) can be used. A composition may contain from about 0.1% to about 10% of sweetener, in particular from about 0.1% to about 1%, by weight of the composition.

In addition the flavor system may include salivating agents, warming agents, and numbing agents. These agents are present in the compositions at a level of from about 0.001% to about 10%, particularly from about 0.1% to about 1%, by weight of the composition. Suitable salivating agents include Jambus manufactured by Takasago. Examples of warming agents are capsicum and nicotinate esters, such as benzyl nicotinate. Suitable numbing agents include benzocaine, lidocaine, clove bud oil, and ethanol.

Examples of suitable peroxide compounds used to prepare the oral compositions of the present invention include hydrogen peroxide and organic peroxides including urea peroxide, glyceryl peroxide, benzoyl peroxide and the like. One such peroxide is hydrogen peroxide.

Typically, the peroxide compound can be employed in the composition of the present invention in amounts so that at least about 1% by weight of the composition comprises a peroxide. The peroxide compound may comprise from about 2% to about 30% by weight of the composition. More particularly, the peroxide comprises from about 3% to about 15% by weight of the composition. A typical peroxide concentration in the composition is generally about 2% to 7% by weight for home use products and about 15% to 20% for dental professional use.

The present invention may include a tooth substantive agent such as polymeric surface active agents (PMSA), which are polyelectrolytes, more specifically anionic polymers. The PMSA contain anionic groups, e.g., phosphate, phosphonate, carboxy, or mixtures thereof, and thus, have the capability to interact with cationic or positively charged entities. The “mineral” descriptor is intended to convey that the surface activity or substantivity of the polymer is toward mineral surfaces such as calcium phosphate minerals or teeth.

PMSAs are useful in the present compositions because of their stain prevention benefit. It is believed the PMSAs provide a stain prevention benefit because of their reactivity or substantivity to mineral surfaces, resulting in desorption of portions of undesirable adsorbed pellicle proteins, in particular those associated with binding color bodies that stain teeth, calculus development and attraction of undesirable microbial species. The retention of these PMSAs on teeth can also prevent stains from accruing due to disruption of binding sites of color bodies on tooth surfaces.

The ability of PMSA to bind stain promoting ingredients of oral care products such as stannous ions and cationic antimicrobials is also believed to be helpful. The PMSA will also provide tooth surface conditioning effects which produce desirable effects on surface thermodynamic properties and surface film properties, which impart improved clean feel aesthetics both during and most importantly, following rinsing or brushing. Many of these polymeric agents are also known or expected to provide tartar control benefits when applied in oral compositions, hence providing improvement in both the appearance of teeth and their tactile impression to consumers.

The desired surface effects include: 1) creating a hydrophilic tooth surface immediately after treatment; and 2) maintaining surface conditioning effects and control of pellicle film for extended periods following product use, including post brushing or rinsing and throughout more extended periods. The effect of creating an increased hydrophilic surface can be measured in terms of a relative decrease in water contact angles. The hydrophilic surface, importantly, is maintained on the tooth surface for an extended period after using the product. \

The polymeric mineral surface active agents include any agent which will have a strong affinity for the tooth surface, deposit a polymer layer or coating on the tooth surface and produce the desired surface modification effects. Suitable examples of such polymers are polyelectrolytes such as condensed phosphorylated polymers; polyphosphonates; copolymers of phosphate- or phosphonate-containing monomers or polymers with other monomers such as ethylenically unsaturated monomers and amino acids or with other polymers such as proteins, polypeptides, polysaccharides, poly(acrylate), poly(acrylamide), poly(methacrylate), poly(ethacrylate), poly(hydroxyalkylmethacrylate), poly(vinyl alcohol), poly(maleic anhydride), poly(maleate) poly(amide), poly(ethylene amine), poly(ethylene glycol), polypropylene glycol), poly(vinyl acetate) and poly(vinyl benzyl chloride); polycarboxylates and carboxy-substituted polymers; and mixtures thereof. Suitable polymeric mineral surface active agents include the carboxy-substituted alcohol polymers described in U.S. Pat. Nos. 5,292,501; 5,213,789, 5,093,170; 5,009,882; and 4,939,284; and the diphosphonate-derivatized polymers in U.S. Pat. No. 5,011,913; the synthetic anionic polymers including polyacrylates and copolymers of maleic anhydride or acid and methyl vinyl ether (e.g., Gantrez), as described, for example, in U.S. Pat. No. 4,627,977. An example of a polymer is diphosphonate modified polyacrylic acid. Polymers with activity must have sufficient surface binding propensity to desorb pellicle proteins and remain affixed to enamel surfaces. For tooth surfaces, polymers with end or side chain phosphate or phosphonate functions may be used, although other polymers with mineral binding activity may prove effective depending upon adsorption affinity.

Agents which chelate metal ions may be used in present invention. The chelating agents are comprised of a blend of chelating agents which include metal solubilizing agents and metal precipitating agents. The metal solubilizing agents include a condensed pyrophosphate compound. For purposes of this invention “condensed phosphate” relates to an inorganic phosphate composition containing two or more phosphate species in a linear or cyclic pyrophosphate form. The condensed phosphate may be sodium pyrophosphate, but may also include tripolyphosphate, hexametaphosphate, cyclic condensed phosphate or other similar phosphates well known in the field. The blend also includes an organic chelating agent. The term “organic phosphate” includes phosphonic acid, di and tri phosphonoc acid compound or its salts. An examples of a phosphonic acid is sold under the trade name of Dequest® 2010 and is called 1-hydroxyethylidene-1,1-diphosphonic acid. The blend also includes a metal precipitating chelating agent. The term “metal precipitating chelating agent” is an agent that binds to metals and causes the metal to precipitate and includes halogens such as fluoride. The chelating agents are incorporated in the oral care compositions of the present invention in an amount within the range of 0.1% to about 8.0% by weight and particularly about 0.5% to about 3.0% by weight in a ratio of about 3:1:1 w/w organic chelating agent: condensed phosphate chelating agent: metal precipitating agent.

Another optional carrier material of the present compositions is a humectant. For example, a humectant may be added to keep toothpaste compositions from hardening upon exposure to air, to give compositions a moist feel to the mouth, and, for particular humectants, to impart desirable sweetness of flavor to toothpaste compositions. The humectant, on a pure humectant basis, generally comprises from about 0% to about 70%, particularly from about 5% to about 25%, by weight of the compositions herein. Suitable humectants for use in compositions of the subject invention include edible polyhydric alcohols such as glycerin, sorbitol, xylitol, butylene glycol, polyethylene glycol, propylene glycol and trimethyl glycine.

These formulations may have a liquid or liquid-like carrier that aides to distribute, disperse, and/or dissolve the formulation ingredients. Selection of these carriers is not limited, and examples of liquid carriers include water, alcohols, oils, esters, and blends thereof.

Denture adhesives augment the same retentive mechanisms already operating when a denture is worn. They enhance retention through optimizing interfacial forces by: 1) increasing the adhesive and cohesive properties and viscosity of the medium lying between the denture and its basal seat; and 2) eliminating voids between the denture base and its basal seat. Adhesives (or, more accurately, the hydrated material that is formed when an adhesive comes into contact with saliva or water) are agents that stick readily to both the tissue surface of the denture and to the mucosal surface of the basal seat. Furthermore, since hydrated adhesives are more cohesive than saliva, physical forces intrinsic to the interposed adhesive medium resist the pull more successfully than would similar forces within saliva. The material increases the viscosity of the saliva with which it mixes, and the hydrated material swells in the presence of saliva/water and flows under pressure. Voids between the denture base and bearing tissues are therefore obliterated.

Synthetic materials presently dominate the denture adhesive market. The most popular and successful products consist of mixtures of the salts of short-acting (carboxymethylcellulose or “CMC”) and long-acting (poly[vinyl methyl ether maleate], or “gantrez” and its salts) polymers. Polyvinylpyrrolidone (“povidone”) is another, less-commonly used agent that behaves like CMC.

Other components of denture adhesive products impart particular physical attributes to the formulations. Petrolatum, mineral oil, and polyethylene oxide are included in creams to bind the materials and to make their placement easier. Silicone dioxide and calcium stearate are used in powders to minimize clumping. Menthol and peppermint oils are used for flavoring, red dye for color, and sodium borate and methyl- or poly-paraben as preservatives.

Formulations according to the invention also may be created for hair, skin, or sun care use. As such, the compositions of the invention also can contain one or more additional additives chosen from conditioning agents, protecting agents, such as, for example, hydrosoluble, antiradical agents, antimicrobials, abrasives, antifungals, botanicals, essential oils, plant extracts, emulsifiers (including but not limited to ethoxylated fatty acids, ethoxylated glyceryl esters, ethoxylated oils, ethoxylated sorbitan esters, fatty esters, PEG esters, polylycerol esters), antiperspirants (such as aluminium chlorohydrates, aluminium zirconium chlorhydrates), antioxidants, anti-fungals (such as pyrrithone), vitamins, ultraviolet absorbers, and pro-vitamins, fixing agents, oxidizing agents, reducing agents, dyes, cleansing agents, anionic, cationic, nonionic and amphoteric surfactants, thickeners, perfumes, pearlizing agents, stabilizers, pH adjusters, filters, preservatives, cationic and nonionic polyether associative polyurethanes, polymers other than the cationic polymer described herein, vegetable oils, mineral oils, synthetic oils, polyols such as glycols and glycerol, silicones, aliphatic alcohols, colorants, bleaching agents, highlighting agents, fragrances, anti-perspirants, propellants (such as hydrocarbons, dimethyl ether, fluorocarbons), hair fixatives, styling polymers, skin benefit agents, skin lighteners (including arbutin and kojic acids), skin tanning agents (including dihydroxyacetone), and sequestrants. These additives are present in the composition according to the invention in proportions that may range from 0% to 20% by weight in relation to the total weight of the composition. The precise amount of each additive may be easily determined by an expert in the field according to its nature and its function.

When the final product aims to protect the user from ultraviolet radiation (such as a sun block or sun screen), it may be desirable to include one or more organic and/or inorganic UV absorbers. In this context, the terms “ultraviolet” and “UV” refer to electromagnetic radiation, especially solar electromagnetic radiation, with a wavelength from about 100 nm to about 400 nm, and includes the UV-A, UV-B, and UV-C subclassifications of such radiation. The term “UV-A” refers to ultraviolet electromagnetic radiation with a wavelength from about 320 nm to about 400 nm, and includes UV-A1 (from about 340 nm to about 400 nm) and UV-A2 (from about 320 nm to about 340 nm).

The term “UV-B” refers to ultraviolet electromagnetic radiation with a wavelength from about 290 nm to about 320 nm. The term “UV-C” reers to ultraviolet electromagnetic radiation with a wavelength from about 200 nm to about 290 nm. Finally, the term “UV absorber” refers to any entity that absorbs, scatters, and/or reflects any wavelength of UV radiation.

Suitable UV absorbers that may be included in the topical compositions and uses of the invention most likely will depend on local regulations. Because the rules governing the names and usage levels evolve over time, it is impossible to include every UV absorber that may be used with the invention. Typical UV absorbers include, without limitation: octyl salicylate; pentyl dimethyl PABA; octyl dimethyl PABA; benzophenone-1; benzophenone-6; 2-(2H-benzotriazole-2-yl)-4,6-di-tert-pentylphenol; ethyl-2-cyano-3,3-diphenylacrylate; homomethyl salicylate; bis-ethylhexyloxyphenol methoxyphenyl triazine; methyl-(1,2,2,6,6-pentamethyl-4-piperidyl)-sebacate; 2-(2H-benzotriazole-2-yl)-4-methylphenol; diethylhexyl butamido triazone; amyl dimethyl PABA; 4,6-bis(octylthiomethyl)-o-cresol; CAS number 65447-77-0; red petroleum; ethylhexyl triazone; octocrylene; isoamyl-p-methoxycinnamate; drometrizole; titanium dioxide; 2,4-di-tert-butyl-6-(5-chloro-2H-benzotriazole-2-yl)-phenol; 2-hydroxy-4-octyloxybenzophenone; benzophenone-2; diisopropyl methylcinnamate; PEG-25 PABA; 2-(1,1-dimethylethyl)-6-[[3-(1,1-demethylethyl)-2-hydroxy-5-methylphenyl]methyl-4-methylphenyl acrylate; drometrizole trisiloxane; menthyl anthranilate; butyl methoxydibenzoylmethane; 2-ethoxyethyl p-methoxycinnamate; benzylidene camphor sulfonic acid; dimethoxyphenyl-[1-(3,4)]-4,4-dimethyl 1,3-pentanedione; zinc oxide; N,N′-hexane-1,6-diylbis[3-(3,5-di-tert-butyl-4-hydroxyphenylpropionamide)]; pentaerythritol tetrakis[3-(3,5-di-tert-butyl-4-hydroxyphenyl)propionate]; 2,6-di-tert-butyl-4-[4,6-bis(octylthio)-1,3,5-triazin-2-ylamino]phenol; 2-(2H-benzotriazole-2-yl)-4,6-bis(1-methyl-1-phenylethyl)phenol; trolamine salicylate; diethylanolamine p-methoxycinnamate; polysilicone-15; CAS number 152261-33-1; 4-methylbenzylidene camphor; bisoctrizole; N-phenyl-benzenamine; reaction products with 2,4,4-trimethylpentene; sulisobenzone; (2-ethylhexyl)-2-cyano-3,3-diphenylacrylate; digalloyl trioleate; polyacrylamido methylbenzylidene camphor; glyceryl ethylhexanoate dimethoxycinnamate; 1,3-bis-[(2′-cyano-3′,3′-diphenylacryloyl)oxy]-2,2-bis-{[(2′-cyano-bis-(2,2,6,6-tetramethyl-4-piperidyl)-sebacate; benzophenone-5; 1,3,5-tris(3,5-di-tert-butyl-4-hydroxybenzyl)-1,3,5-triazine-2,4,6(1H,3H,5H)-trione; hexamethylendiamine; benzophenone-8; ethyl-4-bis(hydroxypropyl)aminobenzoate; 6-tert-butyl-2-(5-chloro-2H-benzotriazole-2-yl)-4-methylphenol; p-aminobenzoic acid; 3,3′,3″,5,5′,5″-hexa-tert-butyl-α-α′-α″-(mesitylene-2,4,6-triyl)tri-p-cresol; lawsone with dihydroxyacetone; benzophenone-9; benzophenone-4; ethylhexyl dimethoxy benzylidene dioxoimidazoline propionate; N,N′-bisformyl-N,N′-bis-(2,2,6,6-tetramethyl-4-piperidinyl)-; 3-benzylidene camphor; terephthalylidene dicamphor sulfonic acid; camphor benzalkonium methosulfate; bisdisulizole disodium; etocrylene; ferulic acid; 2-(2H-benzotriazole-2-yl)-4-(1,1,3,3-tetramethylbutyl)-phenol; 4,6-bis(dodecylthiomethyl)-o-cresol; β-2-glucopyranoxy propyl hydroxy benzophenone; phenylbenzimidazole sulfonic acid; benzophenone-3; diethylamine hydroxybenzoyl hexylbenzoate; 3′,3′-diphenylacryloyl)oxy]methyl}-propane; ethylhexyl p-methoxycinnamate, and blends thereof.

For example, the compositions according to the invention may be used to moisturize, soothe, retain moisture, and/or smooth skin, especially skin of the hands, elbows, and feet, and around the eyes and mouth. In one embodiment the thickened formulations are non-greasy, such as lotions having glycerin, caprylic/capric triglycerides, hydrogenated cocoglycerides, and/or one or more vegetable oils (e.g., helianthus oil, soybean oil, linseed oil, and olive oil).

Any known conditioning agent is useful in the personal care compositions of this invention. Conditioning agents function to improve the cosmetic properties of the hair, particularly softness, thickening, untangling, feel, and static electricity and may be in liquid, semi-solid, or solid form such as oils, waxes, or gums. Similarly, any known skin altering agent is useful in the compositions of this invention. Conditioning agents include cationic polymers, cationic surfactants and cationic silicones.

Conditioning agents may be chosen from synthesis oils, mineral oils, vegetable oils, fluorinated or perfluorinated oils, natural or synthetic waxes, silicones, cationic polymers, proteins and hydrolyzed proteins, ceramide type compounds, cationic surfactants, fatty amines, fatty acids and their derivatives, as well as mixtures of these different compounds.

The synthesis oils include polyolefins, e.g., poly-α-olefins such as polybutenes, polyisobutenes and polydecenes. The polyolefins can be hydrogenated.

The mineral oils suitable for use in the compositions of the invention include hexadecane and oil of paraffin.

A list of suitable animal and vegetable oils comprises sunflower, corn, soy, avocado, jojoba, squash, raisin seed, sesame seed, walnut oils, fish oils, glycerol tricaprocaprylate, Purcellin oil or liquid jojoba, and blends thereof.

Suitable natural or synthetic oils include eucalyptus, lavender, vetiver, litsea cubeba, lemon, sandalwood, rosemary, chamomile, savory, nutmeg, cinnamon, hyssop, caraway, orange, geranium, cade, and bergamot.

Suitable natural and synthetic waxes include carnauba wax, candelila wax, alfa wax, paraffin wax, ozokerite wax, vegetable waxes such as olive wax, rice wax, hydrogenated jojoba wax, absolute flower waxes such as black currant flower wax, animal waxes such as bees wax, modified bees wax (cerabellina), marine waxes and polyolefin waxes such as polyethylene wax, and blends thereof.

The cationic polymers that may be used as a conditioning agent according to the invention are those known to improve the cosmetic properties of hair treated by detergent compositions. The expression “cationic polymer” as used herein, indicates any polymer containing cationic groups and/or ionizable groups in cationic groups. The cationic polymers used generally have a molecular weight the average number of which falls between about 500 Da and 5,000,000 Da and particularly between 1,000 Da and 3,000,000 Da.

The cationic polymers may be chosen from among those containing units including primary, secondary, tertiary, and/or quaternary amine groups that may either form part of the main polymer chain or a side chain.

Useful cationic polymers include known polyamine, polyaminoamide, and quaternary polyammonium types of polymers, such as:
(1) homopolymers and copolymers derived from acrylic or methacrylic esters or amides. The copolymers can contain one or more units derived from acrylamides, methacrylamides, diacetone acrylamides, acrylamides and methacrylamides, acrylic or methacrylic acids or their esters, vinyllactams such as vinyl pyrrolidone or vinyl caprolactam, and vinyl esters. Specific examples include: copolymers of acrylamide and dimethyl amino ethyl methacrylate quaternized with dimethyl sulfate or with an alkyl halide; copolymers of acrylamide and methacryloyl oxyethyl trimethyl ammonium chloride; the copolymer of acrylamide and methacryloyl oxyethyl trimethyl ammonium methosulfate; copolymers of vinyl pyrrolidone/dialkylaminoalkyl acrylate or methacrylate, optionally quaternized, such as the products sold under the name Gafquat® by International Specialty Products; the dimethyl amino ethyl methacrylate/vinyl caprolactam/vinyl pyrrolidone terpolymers, such as the product sold under the name Gaffix® VC 713 by International Specialty Products; the vinyl pyrrolidone/methacrylamidopropyl dimethylamine copolymer, marketed under the name Styleze® CC 10 by International Specialty Products; and the vinyl pyrrolidone/quaternized dimethyl amino propyl methacrylamide copolymers such as the product sold under the name Gafquat® HS 100 by International Specialty Products (Wayne, N.J.).
(2) derivatives of cellulose ethers containing quaternary ammonium groups, such as hydroxy ethyl cellulose quaternary ammonium that has reacted with an epoxide substituted by a trimethyl ammonium group.
(3) derivatives of cationic cellulose such as cellulose copolymers or derivatives of cellulose grafted with a hydrosoluble quaternary ammonium monomer, as described in U.S. Pat. No. 4,131,576, such as the hydroxy alkyl cellulose, and the hydroxymethyl-, hydroxyethyl- or hydroxypropyl-cellulose grafted with a salt of methacryloyl ethyl trimethyl ammonium, methacrylamidopropyl trimethyl ammonium, or dimethyl diallyl ammonium.
(4) cationic polysaccharides such as described in U.S. Pat. Nos. 3,589,578 and 4,031,307, guar gums containing cationic trialkyl ammonium groups and guar gums modified by a salt, e.g., chloride of 2,3-epoxy propyl trimethyl ammonium.
(5) polymers composed of piperazinyl units and alkylene or hydroxy alkylene divalent radicals with straight or branched chains, possibly interrupted by atoms of oxygen, sulfur, nitrogen, or by aromatic or heterocyclic cycles, as well as the products of the oxidation and/or quaternization of such polymers.
(6) water-soluble polyamino amides prepared by polycondensation of an acid compound with a polyamine. These polyamino amides may be reticulated.
(7) derivatives of polyamino amides resulting from the condensation of polyalcoylene polyamines with polycarboxylic acids followed by alcoylation by bi-functional agents.
(8) polymers obtained by reaction of a polyalkylene polyamine containing two primary amine groups and at least one secondary amine group with a dioxycarboxylic acid chosen from among diglycolic acid and saturated dicarboxylic aliphatic acids having 3 to 8 atoms of carbon. Such polymers are described in U.S. Pat. Nos. 3,227,615 and 2,961,347.
(9) the cyclopolymers of alkyl dialyl amine or dialkyl diallyl ammonium such as the homopolymer of dimethyl diallyl ammonium chloride and copolymers of diallyl dimethyl ammonium chloride and acrylamide.
(10) quaternary diammonium polymers such as hexadimethrine chloride.
(11) quaternary polyammonium polymers, including, for example, Mirapol® A 15, Mirapol® AD1, Mirapol® AZ1, and Mirapol® 175 products sold by Mirapol.
(12) the quaternary polymers of vinyl pyrrolidone and vinyl imidazole such as the products sold under the names Luviquat® FC 905, FC 550, and FC 370 by BASF Corporation.
(13) quaternary polyamines.
(14) reticulated polymers known in the art.

Other cationic polymers that may be used within the context of the invention are cationic proteins or hydrolyzed cationic proteins, polyalkyleneimines such as polyethyleneimines, polymers containing vinyl pyridine or vinyl pyridinium units, condensates of polyamines and epichlorhydrins, quaternary polyurethanes, and derivatives of chitin.

The cationic polymers may be derivatives of quaternary cellulose ethers, the homopolymers and copolymers of dimethyl diallyl ammonium chloride, quaternary polymers of vinyl pyrrolidone and vinyl imidazole, and mixtures thereof.

The conditioning agent can be any silicone known by those skilled in the art to be useful as a conditioning agent. The silicones suitable for use according to the invention include polyorganosiloxanes that are insoluble in the composition. The silicones may be present in the form of oils, waxes, resins, or gums. They may be volatile or non-volatile. The silicones can be selected from polyalkyl siloxanes, polyaryl siloxanes, polyalkyl aryl siloxanes, silicone gums and resins, and polyorgano siloxanes modified by organofunctional groups, and mixtures thereof.

Suitable polyalkyl siloxanes include polydimethyl siloxanes with terminal trimethyl silyl groups or terminal dimethyl silanol groups (dimethiconol) and polyalkyl (C1-C20) siloxanes.

Suitable polyalkyl aryl siloxanes include polydimethyl methyl phenyl siloxanes and polydimethyl diphenyl siloxanes, linear or branched.

The silicone gums suitable for use herein include polydiorganosiloxanes, such as those having a number-average molecular weight between 200,000 Da and 1,000,000, Da used alone or mixed with a solvent. Examples include polymethyl siloxane, polydimethyl siloxane/methyl vinyl siloxane gums, polydimethyl siloxane/diphenyl siloxane, polydimethyl siloxane/phenyl methyl siloxane and polydimethyl siloxane/diphenyl siloxane/methyl vinyl siloxane.

Suitable silicone resins include silicones with a dimethyl/trimethyl siloxane structure and resins of the trimethyl siloxysilicate type.

The organo-modified silicones suitable for use in the invention include silicones such as those previously defined and containing one or more organofunctional groups attached by means of a hydrocarbon radical and grafted siliconated polymers. The organo-modified silicone can be one from the amino functional silicone family.

The silicones may be used in the form of emulsions, nano-emulsions, or micro-emulsions.

The conditioning agent can be a protein or hydrolyzed cationic or non-cationic protein. Examples of these compounds include hydrolyzed collagens having triethyl ammonium groups, hydrolyzed collagens having trimethyl ammonium and trimethyl stearyl ammonium chloride groups, hydrolyzed animal proteins having trimethyl benzyl ammonium groups (benzyltrimonium hydrolyzed animal protein), hydrolyzed proteins having groups of quaternary ammonium on the polypeptide chain, including at least one C1-C18 alkyl.

Hydrolyzed proteins include Croquat L, in which the quaternary ammonium groups include a C12 alkyl group, Croquat M, in which the quaternary ammonium groups include C10-C18 alkyl groups, Croquat S in which the quaternary ammonium groups include a C18 alkyl group and Crotein Q in which the quaternary ammonium groups include at least one C1-C18 alkyl group. These products are sold by Croda.

The conditioning agent can comprise quaternized vegetable proteins such as wheat, corn, or soy proteins such as cocodimonium hydrolyzed wheat protein, laurdimonium hydrolyzed wheat protein and steardimonium hydrolyzed wheat protein, 2-N-stearoyl amino-octadecane-1,3-diol, 2-N-behenoyl amino-octadecane-1,3-diol, 2-N-[2-hydroxy-palmitoyl]-amino-octadecane-1,3-diol, 2-N-stearoyl amino-octadecane-1,3,4-triol, N-stearoyl phytosphingosine, 2-N-palmitoyl amino-hexadecane-1,3-diol, bis-(N-hydroxy ethyl N-cetyl)malonamide, N-(2-hydroxy ethyl)-N-(3-cetoxyl-2-hydroxy propyl)amide of cetylic acid, N-docosanoyl N-methyl-D-glucamine and mixtures of such compounds.

The conditioning agent can be a cationic surfactant such as a salt of a primary, secondary, or tertiary fatty amine, optionally polyoxyalkylenated, a quaternary ammonium salt, a derivative of imadazoline, or an amine oxide. Suitable examples include mono-, di-, or tri-alkyl quaternary ammonium compounds with a counterion such as a chloride, methosulfate, tosylate, etc. including, but not limited to, cetrimonium chloride, dicetyldimonium chloride, behentrimonium methosulfate, and the like. The presence of a quaternary ammonium compound in conjunction with the polymer described above reduces static and enhances combing of hair in the dry state. The polymer also enhances the deposition of the quaternary ammonium compound onto the hair substrate thus enhancing the conditioning effect of hair.

The conditioning agent can be any fatty amine known to be useful as a conditioning agent; e.g. dodecyl, cetyl or stearyl amines, such as stearamidopropyl dimethylamine.

The conditioning agent can be a fatty acid or derivatives thereof known to be useful as conditioning agents. Suitable fatty acids include myristic acid, palmitic acid, stearic acid, behenic acid, oleic acid, linoleic acid, and isostearic acid. The derivatives of fatty acids include carboxylic ester acids including mono-, di-, tri- and tetra-carboxylic acids.

The conditioning agent can be a fluorinated or perfluorinated oil. The fluoridated oils may also be fluorocarbons such as fluoramines, e.g., perfluorotributylamine, fluoridated hydrocarbons, such as perfluorodecahydronaphthalene, fluoroesters, and fluoroethers.

Of course, mixtures of two or more conditioning agents can be used.

The conditioning agent or agents can be present in an amount of 0.001% to 20%, particularly from 0.01% to 10%, and even more particularly from 0.1% to 3% by weight based on the total weight of the final composition.

The antioxidants or antiradical agents can be selected from phenols such as BHA (tert-butyl-4-hydroxy anisole), BHT (2,6-di-tert-butyl-p-cresol), TBHQ (tert-butyl hydroquinone), polyphenols such as proanthocyanodic oligomers, flavonoids, hindered amines such as tetra amino piperidine, erythorbic acid, polyamines such as spermine, cysteine, glutathione, superoxide dismutase, and lactoferrin.

The vitamins can be selected from ascorbic acid (vitamin C), vitamin E, vitamin E acetate, vitamin E phosphate, B vitamins such as B3 and B5, niacin, vitamin A, and derivatives thereof. The provitamins can be selected from panthenol and retinol.

The protecting agent can be present in an amount 0.001% to 20% by weight, particularly from 0.01% to 10% by weight, and more particularly 0.1 to 5% by weight of the total weight of the final composition.

In addition, the compositions according to the invention advantageously include at least one surfactant, which can be present in an amount of 0.1% and 60%, particularly 1% and 40%, and more particularly 5% and 30% by weight based on the total weight of the composition. The surfactant may be chosen from among anionic, amphoteric, or non-ionic surfactants, or mixtures of them known to be useful in personal care and oral care compositions.

Additional thickeners or viscosity increasing agents may be included in the composition of the invention, such as: Acetamide MEA; acrylamide/ethalkonium chloride acrylate copolymer; acrylamide/ethyltrimonium chloride acrylate/ethalkonium chloride acrylate copolymer; acrylamides copolymer; acrylamide/sodium acrylate copolymer; acrylamide/sodium acryloyldimethyltaurate copolymer; acrylates/acetoacetoxyethyl methacrylate copolymer; acrylates/beheneth-25 methacrylate copolymer; acrylates/C10-C30 alkyl acrylate crosspolymer; acrylates/ceteth-20 itaconate copolymer; acrylates/ceteth-20 methacrylate copolymer; acrylates/laureth-25 methacrylate copolymer; acrylates/palmeth-25 acrylate copolymer; acrylates/palmeth-25 itaconate copolymer; acrylates/steareth-50 acrylate copolymer; acrylates/steareth-20 itaconate copolymer; acrylates/steareth-20 methacrylate copolymer; acrylates/stearyl methacrylate copolymer; acrylates/vinyl isodecanoate crosspolymer; acrylic acid/acrylonitrogens copolymer; adipic acid/methyl DEA crosspolymer; agar; agarose; alcaligenes polysaccharides; algin; alginic acid; almondamide DEA; almondamidopropyl betaine; aluminum/magnesium hydroxide stearate; ammonium acrylates/acrylonitrogens copolymer; ammonium acrylates copolymer; ammonium acryloyldimethyltaurate/vinyl formamide copolymer; ammonium acryloyldimethyltaurate/VP copolymer; ammonium alginate; ammonium chloride; ammonium polyacryloyldimethyl taurate; ammonium sulfate; amylopectin; apricotamide DEA; apricotamidopropyl betaine; arachidyl alcohol; arachidyl glycol; arachis hypogaea (peanut) flour; ascorbyl methylsilanol pectinate; astragalus gummifer gum; attapulgite; avena sativa (oat) kernel flour; avocadamide DEA; avocadamidopropyl betaine; azelamide MEA; babassuamide DEA; babassuamide MEA; babassuamidopropyl betaine; behenamide DEA; behenamide MEA; behenamidopropyl betaine; behenyl betaine; bentonite; butoxy chitosan; caesalpinia spinosa gum; calcium alginate; calcium carboxymethyl cellulose; calcium carrageenan; calcium chloride; calcium potassium carbomer; calcium starch octenylsuccinate; C20-40 alkyl stearate; canolamidopropyl betaine; capramide DEA; capryl/capramidopropyl betaine; carbomer; carboxybutyl chitosan; carboxymethyl cellulose acetate butyrate; carboxymethyl chitin; carboxymethyl chitosan; carboxymethyl dextran; carboxymethyl hydroxyethylcellulose; carboxymethyl hydroxypropyl guar; carnitine; cellulose acetate propionate carboxylate; cellulose gum; ceratonia siliqua gum; cetearyl alcohol; cetyl alcohol; cetyl babassuate; cetyl betaine; cetyl glycol; cetyl hydroxyethylcellulose; chimyl alcohol; cholesterol/HDI/pullulan copolymer; cholesteryl hexyl dicarbamate pullulan; citrus aurantium dulcis (orange) peel extract; cocamide DEA; cocamide MEA; cocamide MIPA; cocamidoethyl betaine; cocamidopropyl betaine; cocamidopropyl hydroxysultaine; coco-betaine; coco-hydroxysultaine; coconut alcohol; coco/oleamidopropyl betaine; coco-Sultaine; cocoyl sarcosinamide DEA; cornamide/cocamide DEA; cornamide DEA; croscarmellose; crosslinked bacillus/glucose/sodium glutamate ferment; cyamopsis tetragonoloba (guar) gum; decyl alcohol; decyl betaine; dehydroxanthan gum; dextrin; dibenzylidene sorbitol; diethanolaminooleamide DEA; diglycol/CHDM/isophthalates/SIP copolymer; dihydroabietyl behenate; dihydrogenated tallow benzylmonium hectorite; dihydroxyaluminum amino acetate; dimethicone/PEG-10 crosspolymer; dimethicone/PEG-15 crosspolymer; dimethicone propyl PG-betaine; dimethylacrylamide/acrylic acid/polystyrene ethyl methacrylate copolymer; dimethylacrylamide/sodium acryloyldimethyltaurate crosspolymer; disteareth-100 IPDI; DMAPA acrylates/acrylic acid/acrylonitrogens copolymer; erucamidopropyl hydroxysultaine; ethylene/sodium acrylate copolymer; gelatin; gellan gum; glyceryl alginate; glycine soja (soybean) flour; guar hydroxypropyltrimonium chloride; hectorite; hyaluronic acid; hydrated silica; hydrogenated potato starch; hydrogenated tallow; hydrogenated tallowamide DEA; hydrogenated tallow betaine; hydroxybutyl methylcellulose; hydroxyethyl acrylate/sodium acryloyldimethyl taurate copolymer; hydroxyethylcellulose; hydroxyethyl chitosan; hydroxyethyl ethylcellulose; hydroxyethyl stearamide-MIPA; hydroxylauryl/hydroxymyristyl betaine; hydroxypropylcellulose; hydroxypropyl chitosan; hydroxypropyl ethylene diamine carbomer; hydroxypropyl guar; hydroxypropyl methylcellulose; hydroxypropyl methylcellulose stearoxy ether; hydroxypropyl starch; hydroxypropyl starch phosphate; hydroxypropyl xanthan gum; hydroxystearamide MEA; isobutylene/sodium maleate copolymer; isostearamide DEA; isostearamide MEA; isostearamide mIPA; isostearamidopropyl betaine; lactamide MEA; lanolinamide DEA; lauramide DEA; lauramide MEA; lauramide MIPA; lauramide/myristamide DEA; lauramidopropyl betaine; lauramidopropyl hydroxysultaine; laurimino bispropanediol; lauryl alcohol; lauryl betaine; lauryl hydroxysultaine; lauryl/myristyl glycol hydroxypropyl ether; lauryl sultaine; lecithinamide DEA; linoleamide DEA; linoleamide MEA; linoleamide MIPA; lithium magnesium silicate; lithium magnesium sodium silicate; macrocystis pyrifera (kelp); magnesium alginate; magnesium/aluminum/hydroxide/carbonate; magnesium aluminum silicate; magnesium silicate; magnesium trisilicate; methoxy PEG-22/dodecyl glycol copolymer; methylcellulose; methyl ethylcellulose; methyl hydroxyethylcellulose; microcrystalline cellulose; milkamidopropyl betaine; minkamide DEA; minkamidopropyl betaine; MIPA-myristate; montmorillonite; Moroccan lava clay; myristamide DEA; myristamide MEA; myristamide MIPA; myristamidopropyl betaine; myristamidopropyl hydroxysultaine; myristyl alcohol; myristyl betaine; natto gum; nonoxynyl hydroxyethylcellulose; oatamide MEA; oatamidopropyl betaine; octacosanyl glycol isostearate; octadecene/MA copolymer; oleamide DEA; oleamide MEA; oleamide MIPA; oleamidopropyl betaine; oleamidopropyl hydroxysultaine; oleyl betaine; olivamide DEA; olivamidopropyl betaine; oliveamide MEA; palmamide DEA; palmamide MEA; palmamide MIPA; palmamidopropyl betaine; palmitamide DEA; palmitamide MEA; palmitamidopropyl betaine; palm kernel alcohol; palm kernelamide DEA; palm kernelamide MEA; palm kernelamide MIPA; palm kernelamidopropyl betaine; peanutamide MEA; peanutamide MIPA; pectin; PEG-800; PEG-crosspolymer; PEG-150/decyl alcohol/SMDI copolymer; PEG-175 diisostearate; PEG-190 distearate; PEG-15 glyceryl tristearate; PEG-140 glyceryl tristearate; PEG-240/HDI copolymer bis-decyltetradeceth-20 ether; PEG-100/IPDI copolymer; PEG-180/laureth-50/TMMG copolymer; PEG-10/lauryl dimethicone crosspolymer; PEG-15/lauryl dimethicone crosspolymer; PEG-2M; PEG-5M; PEG-7M; PEG-9M; PEG-14M; PEG-20M; PEG-23M; PEG-25M; PEG-45M; PEG-65M; PEG-90M; PEG-115M; PEG-160M; PEG-180M; PEG-120 methyl glucose trioleate; PEG-180/octoxynol-40/TMMG copolymer; PEG-150 pentaerythrityl tetrastearate; PEG-4 rapeseedamide; PEG-150/stearyl alcohol/SMDI copolymer; phaseolus angularis seed powder; polianthes tuberosa extract; polyacrylate-3; polyacrylic acid; polycyclopentadiene; polyether-1; polyethylene/isopropyl maleate/MA copolyol; polyglyceryl-3 disiloxane dimethicone; polyglyceryl-3 polydimethylsiloxyethyl dimethicone; polymethacrylic acid; polyquaternium-52; polyvinyl alcohol; potassium alginate; potassium aluminum polyacrylate; potassium carbomer; potassium carrageenan; potassium chloride; potassium palmate; potassium polyacrylate; potassium sulfate; potato starch modified; PPG-2 cocamide; PPG-1 hydroxyethyl caprylamide; PPG-2 hydroxyethyl cocamide; PPG-2 hydroxyethyl coco/isostearamide; PPG-3 hydroxyethyl soyamide; PPG-14 laureth-60 hexyl dicarbamate; PPG-14 laureth-60 isophoryl dicarbamate; PPG-14 palmeth-60 hexyl dicarbamate; propylene glycol alginate; PVP/decene copolymer; PVP montmorillonite; pyrus cydonia seed; pyrus malus (apple) fiber; rhizobian gum; ricebranamide DEA; ricinoleamide DEA; ricinoleamide MEA; ricinoleamide MIPA; ricinoleamidopropyl betaine; ricinoleic acid/adipic acid/AEEA copolymer; rosa multiflora flower wax; sclerotium gum; sesamide DEA; sesamidopropyl betaine; sodium acrylate/acryloyldimethyl taurate copolymer; sodium acrylates/acrolein copolymer; sodium acrylates/acrylonitrogens copolymer; sodium acrylates copolymer; sodium acrylates crosspolymer; sodium acrylate/sodium acrylamidomethylpropane sulfonate copolymer; sodium acrylates/vinyl isodecanoate crosspolymer; sodium acrylate/vinyl alcohol copolymer; sodium carbomer; sodium carboxymethyl chitin; sodium carboxymethyl dextran; sodium carboxymethyl beta-glucan; sodium carboxymethyl starch; sodium carrageenan; sodium cellulose sulfate; sodium chloride; sodium cyclodextrin sulfate; sodium hydroxypropyl starch phosphate; sodium isooctylene/MA copolymer; sodium magnesium fluorosilicate; sodium oleate; sodium palmitate; sodium palm kernelate; sodium polyacrylate; sodium polyacrylate starch; sodium polyacryloyldimethyl taurate; sodium polygamma-glutamate; sodium polymethacrylate; sodium polystyrene sulfonate; sodium silicoaluminate; sodium starch octenylsuccinate; sodium stearate; sodium stearoxy PG-hydroxyethylcellulose sulfonate; sodium styrene/acrylates copolymer; sodium sulfate; sodium tallowate; sodium tauride acrylates/acrylic acid/acrylonitrogens copolymer; sodium tocopheryl phosphate; solanum tuberosum (potato) starch; soyamide DEA; soyamidopropyl betaine; starch/acrylates/acrylamide copolymer; starch hydroxypropyltrimonium chloride; stearamide AMP; stearamide DEA; stearamide DEA-distearate; stearamide DIBA-stearate; stearamide MEA; stearamide MEA-stearate; stearamide MIPA; stearamidopropyl betaine; steareth-60 cetyl ether; steareth-100/PEG-136/HDI copolymer; stearyl alcohol; stearyl betaine; sterculia urens gum; synthetic fluorphlogopite; tallamide DEA; tallow alcohol; tallowamide DEA; tallowamide MEA; tallowamidopropyl betaine; tallowamidopropyl hydroxysultaine; tallowamine oxide; tallow betaine; tallow dihydroxyethyl betaine; tamarindus indica seed gum; tapioca starch; TEA-alginate; TEA-carbomer; TEA-hydrochloride; trideceth-2 carboxamide MEA; tridecyl alcohol; triethylene glycol dibenzoate; trimethyl pentanol hydroxyethyl ether; triticum vulgare (wheat) germ powder; triticum vulgare (wheat) kernel flour; triticum vulgare (wheat) starch; tromethamine acrylates/acrylonitrogens copolymer; tromethamine magnesium aluminum silicate; undecyl alcohol; undecylenamide DEA; undecylenamide MEA; undecylenamidopropyl betaine; welan gum; wheat germamide DEA; wheat germamidopropyl betaine; xanthan gum; yeast beta-glucan; yeast polysaccharides and zea mays (corn) starch.

### Product Forms

Acknowledging the many ways compositions of the invention may be used, it is within the scope of the invention that the compositions may take the form of a solution, a cream, an ointment, a lotion, an oil-in-water emulsion, a water-in-oil emulsion, a shampoo, a spray, a gel, a wash, a rinse, an aerosol, a mist, a suspension, a paste, a powder, a serum, or a mousse.

The compositions may be used, for example, to style, enhance, rejuvenate, fix, maintain, and/or hold hair styling and appearance. Hair care compositions may take the form of a spray, mist, mousse, gel, cream, tonic, wax, pomade, serum, or lotion.

When the invention's compositions are used in oral care, it is expected that the product form may be a paste, a gel, a liquid, or combinations thereof.

In other examples of the invention, the invention's compositions may be used to wash and treat keratinous material such as hair, skin, eyelashes, eyebrows, fingernails, lips, and hairy skin. The compositions of the invention may also take the form of skin-washing compositions, and particularly in the form of solutions or gels for the bath or shower, or of make-up removal products.

The compositions according to the invention may also take the form of after-shampoo compositions, to be rinsed off or not, for permanents, straightening, waving, dyeing, or bleaching, or the form of rinse compositions to be applied before or after dyeing, bleaching, permanents, straightening, relaxing, waving or even between the two stages of a permanent or straightening process.

Examples of related compositions are disclosed in U.S. Pat. Nos. 5,599,800; 5,650,166; 5,916,549; and 6,812,192; U.S. patent application 2009/0317432; EP 556,660; 661,037; 661,038; 662,315; 676,194; 796,077; 970,682; 976383; 1,415,654; and 2,067,467; and WO 2005/032506; each of which is incorporated herein its entirety by reference.

The compositions according to the invention can be detergent compositions such as shampoos, bath gels, and bubble baths. In this mode, the compositions will comprise water as a liquid carrier. The surfactant or surfactants that form the washing base may be chosen alone or in blends, from known anionic, amphoteric, or non-ionic surfactants. The quantity and quality of the washing base must be sufficient to impart a satisfactory foaming and/or detergent value to the final composition. The washing base can be from 4% to 50% by weight, particularly from 6% to 35% by weight, and even more particularly from 8% to 25% by weight of the total weight of the final composition.

Cosmetic compositions according to the invention may, for example, be used as care and/or sun protection product for the face and/or the body having a consistency ranging from liquid to semiliquid (e.g., milks, creams), and gels, creams, pastes, powders (including compacted powders), and wax-like compositions (e.g., lip balms).

For compositions intended to protect the hair from UV radiation, suitable product forms include, but not limited to: conditioners, dispersions, emulsions, gels, lotions, mists, mousses, shampoos, and sprays.

The personal care active includes shampoo, body wash products, shaving cream, hand soap, bubble bath, bath gel, after-shave lotions, creams, moisturizers, sunscreens, liquid soaps, color cosmetics, acid peels, perms, hair color, sunless tanning and conditioners.

### EXAMPLES

### Example 1

### Synthesis of Branched PVP with 0.7% DVFOO Branching Agent

In a 1-L resin kettle equipped with an anchor agitator, thermocouple, gas inlet and reflux condenser, 100 g of N-vinyl-2-pyrrolidone, 200 g of water and 0.7 g of 1,8-di-(N-vinyl formamido)-3,6-dioxyoctane (DVFOO) were charged. The reaction mixture was purged with nitrogen for 30 minutes and then 0.649 g of a 26% NH₄OH aqueous solution was added. With agitation and nitrogen purging, the reactor was heated to 65° C., then 1.79 g of a 35% H₂O₂ aqueous solution was added followed by 0.01 g of a 0.12% CuSO₄ aqueous solution. After 15 minutes of reaction, 0.67 g of a 35% H₂O₂ aqueous solution was charged into the reactor. The reaction was held for 15 minutes, and then 0.55 g of a 35% H₂O₂ aqueous solution was charged into the reactor. After 3 hours, the reaction mixture was cooled to room temperature to discharge the product.

Gas chromatography (GC) was used to measure the residual monomer for this and all Examples that follow. In this polymer the residual N-vinyl-2-pyrrolidone content was less than 100 ppm. The K-value of a 1% aqueous solution of this polymer was 37.7. The molecular weights for this Example and all that follow were measured by size-exclusion chromatography using a 25,000 Da poly(oxyethylene) standard. The weight-average molecular weight (M_{w}) was 229,000 Da, and the number-average molecular weight (Mₙ) was 39,110 Da.

### Example 2

### Synthesis of Branched PVP with 0.7% DVFOO Branching Agent

In a 1-L resin kettle equipped with an anchor agitator, thermocouple, gas inlet, reflux condenser and nitrogen blanket, 75.0 g of N-vinyl-2-pyrrolidone, 200 g of water and 0.7 g of DVFOO were charged. The reaction mixture was purged with nitrogen for 30 minutes and 0.649 g of a 26% NH₄OH aqueous solution was added. Afterward, a charge of 25.0 g of N-vinyl-2-pyrrolidone was added, bringing the total amount of this monomer to 100.0 g. The reactor was heated to 65° C. then 1.887 g of H₂O₂ was added followed by 0.01 g of a 0.12% CuSO₄ aqueous solution. After 15 minutes, 25 g of N-vinyl-2-pyrrolidone were added and temperature increased to 87° C. When the temperature dropped to 80° C., the reaction was maintained for 0.5 hour and 0.67 g of a 35% H₂O₂ aqueous solution was charged. After 15 minutes, 0.55 g of a 35% H₂O₂ aqueous solution was added into the reactor. The reaction was maintained for 3 hours then cooled to room temperature to discharge the product.

The residual N-vinyl-2-pyrrolidone content in the polymer was 340 ppm. The K-value of this polymer in 1% aqueous solution was 42.7. M_{w} was found to be 243,400 Da, and Mₙ was 35,210 Da.

### Example 3

### Synthesis of Branched PVP with 0.7% DVFOO Branching Agent

Example 2 was repeated.

The residual N-vinyl-2-pyrrolidone content in the polymer was 112 ppm. The K-value of this polymer in 1% aqueous solution was 46.1. M_{w} was found to be 391,410 Da, and Mₙ was 37,181 Da.

### Example 4

### Synthesis of Branched PVP with 0.85% DVFOO Branching Agent

Example 1 was substantially repeated, with the exception that 0.85 g (instead of 0.7 g) of DVFOO was charged.

The residual N-vinyl-2-pyrrolidone content in the polymer was less than 100 ppm. The K-value of this polymer in 1% aqueous solution was 42.2. M_{w} was found to be 372,000 Da, and Mₙ was 46,470 Da.

### Example 5

### Synthesis of Branched PVP with 0.85% DVFOO Branching Agent

Example 4 was repeated.

The residual N-vinyl-2-pyrrolidone content in the polymer was less than 100 ppm. The K-value of this polymer in 1% aqueous solution was 41.1. M_{w} was found to be 441,000 Da, and Mₙ was 59,740 Da.

### Example 6

### Synthesis of Branched PVP with 1% DVFOO Branching Agent

Example 1 was substantially repeated, with the exception that 1.0 g (instead of 0.7 g) of DVFOO was charged.

The residual N-vinyl-2-pyrrolidone content in the polymer was less than 100 ppm. The K-value of this polymer in 1% aqueous solution was 49.3.

### Example 7

### Synthesis of Branched PVP with 1.0% DVFOO Branching Agent

Example 6 was repeated.

The residual N-vinyl-2-pyrrolidone content in the polymer was less than 60 ppm. The K-value of this polymer in 1% aqueous solution was 43.8. M_{w} was found to be 508,000 Da, and Mₙ was 63,780 Da.

### Example 8

### Synthesis of Branched PVP with 1.0% DVFOO Branching Agent

Example 6 was repeated.

The residual N-vinyl-2-pyrrolidone content in the polymer was less than 60 ppm. The K-value of this polymer in 1% aqueous solution was 50.5. M_{w} was found to be 1,416,000 Da, and Mₙ was 110,600 Da. While this polymer has a mean M_{w} greater than that of PVP K-90 (which has M_{w} of 1,300,000 Da), the polymer of this Example is considerably less viscous, as indicated by its lower K-value. The GPC recovery was 86.7%, suggesting a portion of this polymer was crosslinked.

### Example 9

### Synthesis of Branched PVP with 1.0% DVFOO Branching Agent

Example 7 was substantially repeated, except that a first charge of N-vinyl-2-pyrrolidone was 50.0 g, followed by a second charge of 50.0 g this monomer 30 minutes later.

The residual N-vinyl-2-pyrrolidone content in the polymer was 428 ppm. The K-value of this polymer in 1% aqueous solution was 52.1 M_{w} was found to be 681,740 Da, and Mₙ was 87,600 Da.

### Example 10

### Synthesis of Branched PVP with 1.0% DVFOO Branching Agent

Example 9 was substantially repeated, except that a first charge of N-vinyl-2-pyrrolidone was 75.0 g, followed by a second charge of 25.0 g this monomer 30 minutes later.

The residual N-vinyl-2-pyrrolidone content in the polymer was 271 ppm. The K-value of this polymer in 1% aqueous solution was 43.3. M_{w} was found to be 828,350 Da, and Mₙ was 78,050 Da.

### Example 11

### Synthesis of Branched PVP with 1.0% DVFOO Branching Agent

Example 10 was repeated.

The residual N-vinyl-2-pyrrolidone content in the polymer was 130 ppm. The K-value of this polymer in 1% aqueous solution was 41.1. M_{w} was found to be 450,000 Da, and Mₙ was 57,120 Da.

### Comparative Example 1

### Synthesis of Crosslinked PVP with 1.25% DVFOO

Example 1 was substantially repeated, with the exception that 1.25 g (instead of 0.7 g) of DVFOO was charged.

A water-insoluble, elastic, crosslinked polymer was produced.

### Example 12

### Synthesis of Branched PVP with 1% DVFOO Branching Agent (Non-Isothermal Process)

In a 1-L resin kettle equipped with an anchor agitator, thermocouple, gas inlet and reflux condenser, 100 g of N-vinyl-2-pyrrolidone, 200 g of water and 1.0 g of 1,8-di-(N-vinyl formamido)-3,6-dioxyoctane were charged. The reaction mixture was purged with nitrogen for 30 minutes and 0.649 g of a 26% NH₄OH aqueous solution was added. After vigorous agitation and nitrogen purging, the reactor was heated to 65° C., and then 1.79 g of a 35% H₂O₂ aqueous solution was added followed by 0.01 g of a 0.12% CuSO₄ aqueous solution. After 15 minutes, 0.67 g of a 35% H₂O₂ aqueous solution was charged into the reactor. At this time the reaction was held for 15 minutes, and then 0.55 g of a 35% H₂O₂ aqueous solution was charged. After 3 hours, the reaction mixture was cooled to room temperature to discharge the product.

The residual N-vinyl-2-pyrrolidone content was less than 100 ppm. The K-value of a 1% aqueous solution of this polymer was 38.2. M_{w} was found to be 331,700 Da, and the GPC recovery was 102%. Complete GPC recovery of the polymer indicates negligible or zero crosslinked/gel content.

### Example 13

### Synthesis of Branched PVP with 1% DVFOO Branching Agent (Non-Isothermal Process)

Example 12 was repeated.

The residual N-vinyl-2-pyrrolidone content was less than 100 ppm. The K-value of a 1% aqueous solution of this polymer was 39.6. M_{w} was 371,510 Da, and the GPC recovery was 100%. Complete GPC recovery of the polymer indicates negligible or zero crosslinked/gel content.

### Example 14

### Synthesis of Branched PVP with 1% DVFOO Branching Agent (Non-Isothermal Process)

Example 12 was repeated.

The residual N-vinyl-2-pyrrolidone content was less than 100 ppm. The K-value of a 1% aqueous solution of this polymer was 38.4. M_{w} was 359,330 Da. GPC recovery was 99%, suggesting negligible or zero crosslinked/gel content. The glass transition temperature was found to be 164.9° C., as measured by differential scanning calorimetry.

### Example 15

### Synthesis of Branched PVP with 1% DVFOO Branching Agent and 0.31% Mercaptoethanol Chain-Transfer Agent

In a 1-L resin kettle equipped with an anchor agitator, thermocouple, gas inlet and reflux condenser, 100 g of N-vinyl-2-pyrrolidone, 200 g of water, 0.1 g of 1,8-di-(N-vinyl formamido)-3,6-dioxyoctane and 0.31 g of 2-mercaptoethanol were charged. The reaction mixture was purged with nitrogen for 30 minutes and 0.649 g of a 26% NH₄OH aqueous solution was added. With agitation and nitrogen purging, the reactor was heated to 65° C., and then 1.79 g of a 35% H₂O₂ aqueous solution was added followed by 0.01 g of a 0.12% CuSO₄ aqueous solution. After 15 minutes of reaction, 0.67 g of a 35% H₂O₂ aqueous solution was charged into the reactor. The reaction was held for 15 minutes, and then 0.55 g of a 35% H₂O₂ aqueous solution was charged into the reactor. After 3 hours, the reaction mixture was cooled to room temperature to discharge the product.

The residual N-vinyl-2-pyrrolidone was less than 100 ppm. The K-value of a 1% aqueous solution of this polymer was 48.1. M_{w} was 713,530 Da, and the GPC recovery was 99%, suggesting negligible or zero crosslinked/gel content. The glass transition temperature was found to be 166.5° C., as measured by differential scanning calorimetry.

### Example 16

### Synthesis of Branched PVP with 1% DVFOO Branching Agent and 0.31% Mercaptoethanol Chain-Transfer Agent

Example 15 was repeated.

The residual N-vinyl-2-pyrrolidone was less than 100 ppm. The K-value of a 1% aqueous solution of this polymer was 46.1. M_{w} was 697,160 Da, and the GPC recovery was 98%, suggesting negligible or zero crosslinked/gel content. The glass transition temperature was found to be 168.2° C., as measured by differential scanning calorimetry.

### Example 17

### Synthesis of Branched PVP with 1% DVFOO Branching Agent and 0.31% Mercaptoethanol Chain-Transfer Agent

Example 15 was repeated.

The residual N-vinyl-2-pyrrolidone was less than 100 ppm. The K-value of a 1% aqueous solution of this polymer was 44.4. M_{w} was 936,460 Da, and the GPC recovery was 97%, suggesting negligible or zero crosslinked/gel content.

### Example 18

### Synthesis of Branched PVP with 0.45% DVFH Branching Agent (Isothermal Process)

In a 1-L resin kettle equipped with an anchor agitator, thermocouple, gas inlet and reflux condenser, 200 g of water and 0.9 g of a 26% NH₄OH aqueous solution were added. With agitation and nitrogen purging, the reactor was heated to 80° C., then a feed of 100 g of N-vinyl-2-pyrrolidone and 0.5 g of 1,6-di-(N-vinyl formamido) hexane was added over a period of two hours, and then 0.1 g of a 0.12% CuSO₄ aqueous solution was charged. After 30 minutes of starting the feed, 0.6 g of a 35% H₂O₂ aqueous solution was charged into the reactor. After 30 minutes of reaction time, six shots of 35% H₂O₂ aqueous solution (each shot 1.5 g) were charged into the reactor over the period 3 hours at 80° C., and the reaction was held at this temperature for 4 hours.

The residual N-vinyl-2-pyrrolidone content was less than 100 ppm. The K-value of a 1% aqueous solution of this polymer was 40. M_{w} was 310,000 Da, and the GPC recovery was 99.1%, suggesting negligible or zero crosslinked/gel content.

### Example 19

### Synthesis of Branched PVP with 0.55% DVFH Branching Agent (Isothermal Process)

In a 1-L resin kettle equipped with an anchor agitator, thermocouple, gas inlet and reflux condenser, 200 g of water and 0.9 g of a 26% NH₄OH aqueous solution were added. With agitation and nitrogen purging, the reactor was heated to 80° C., then a feed of 100 g of N-vinyl-2-pyrrolidone and 0.55 g of 1,6-di-(N-vinyl formamido) hexane (DVFH) was added over a period of two hours, followed by 0.1 g of a 0.12% CuSO₄ aqueous solution. After 30 minutes 0.6 g of a 35% H₂O₂ aqueous solution was charged into the reactor. Then, after 30 minutes of reaction, six shots of a 35% H₂O₂ aqueous solution (each shot 1.5 g) were charged into the reactor over the period 3 hours at 80° C., and the reaction was maintained at this temperature for 4 hours.

The residual N-vinyl-2-pyrrolidone content was less than 100 ppm. The K-value of a 1% aqueous solution of this polymer was 50.5. M_{w} was 621,550 Da, and the GPC recovery was 100%. Complete GPC recovery indicates negligible or zero crosslinked/gel content. The glass transition temperature was found to be 164.8° C., as measured by differential scanning calorimetry.

### Example 20

### Synthesis of Branched PVP with 0.55% DVFH Branching Agent (Isothermal Process)

Example 19 was repeated.

The residual N-vinyl-2-pyrrolidone content was less than 100 ppm. The K-value of a 1% aqueous solution of this polymer was 50.5. M_{w} was 679,950 Da, and the GPC recovery was 101%. Complete GPC recovery indicates negligible or zero crosslinked/gel content.

### Example 21

### Synthesis of Branched PVP with 0.55% DVFH Branching Agent (Isothermal Process)

Example 19 was repeated.

The residual N-vinyl-2-pyrrolidone content was less than 100 ppm. The K-value of a 1% aqueous solution of this polymer was 50.5. M_{w} was 649,590 Da, and the GPC recovery was 98%, suggesting negligible or zero crosslinked/gel content.

### Example 22

### Synthesis of Branched PVP with 0.55% DVFH Branching Agent (Isothermal Process)

Example 19 was repeated.

The residual N-vinyl-2-pyrrolidone content was less than 100 ppm. The K-value of a 1% aqueous solution of this polymer was 43.2. M_{w} was 400,000 Da.

### Example 23

### Synthesis of Branched PVP with 0.55% DVFH Branching Agent (Isothermal Process)

Example 19 was repeated.

The residual N-vinyl-2-pyrrolidone content was less than 100 ppm. The K-value of a 1% aqueous solution of this polymer was 46. M_{w} was 877,580 Da, and the GPC recovery was 101%, suggesting negligible or zero crosslinked/gel content.

### Example 24

### Synthesis of Branched PVP with 0.55% DVFH Branching Agent and 0.2% Mercaptoethanol Chain Transfer Agent (Isothermal Process)

In a 1-L resin kettle equipped with an anchor agitator, thermocouple, gas inlet and reflux condenser, 200 g of water and 0.9 g of a 26% NH₄OH aqueous solution was added. With agitation and nitrogen purging, the reactor was heated to 80° C., then a feed of 100 g of N-vinyl-2-pyrrolidone, 0.2 g of 2-mercaptoethanol and 0.55 g of 1,6-di-(N-vinyl formamido) hexane was added over a period of two hours, after which 0.1 g of a 0.12% CuSO₄ aqueous solution was charged. After 30 minutes of starting the feed, 0.6 g of a 35% H₂O₂ aqueous solution was charged into the reactor. After 30 minutes of reaction time, six shots of a 35% H₂O₂ aqueous solution were charged into the reactor every 3 hours at 80° C., and then the reaction was held at this temperature for 4 hours. At this point the reaction mixture was cooled to room temperature to discharge the product.

The residual N-vinyl-2-pyrrolidone content was less than 100 ppm. The K-value of a 1% aqueous solution of this polymer was 41. M_{w} was 293,200 Da, and Mₙ was 90,600 Da. GPC recovery was 96%, suggesting negligible or zero crosslinked/gel content. The glass transition temperature was 164.8° C., as determined by differential scanning calorimetry.

### Example 25

### Synthesis of Branched PVP with 0.55% DVFH Branching Agent and 0.2% Mercaptoethanol Chain Transfer Agent (Isothermal Process)

Example 24 was repeated.

The residual N-vinyl-2-pyrrolidone content was less than 100 ppm. The K-value of a 1% aqueous solution of this polymer was 44. M_{w} was 151,900 Da, and Mₙ was 52,900 Da. GPC recovery was 103%. Complete GPC recovery indicates negligible or zero crosslinked/gel content.

### Example 26

### Synthesis of Branched PVP with 0.55% DVFH Branching Agent and 0.2% Mercaptoethanol Chain Transfer Agent (Isothermal Process)

Example 24 was repeated.

The residual N-vinyl-2-pyrrolidone content was less than 100 ppm. The K-value of a 1% aqueous solution of this polymer was 40.5. M_{w} was 216,910 Da, and Mₙ was 69,800 Da. GPC recovery was 97%, suggesting negligible or zero crosslinked/gel content.

### Comparative Example 2

### Synthesis of Crosslinked PVP with 0.6% DVFH

Example 24 was substantially repeated, with the exception that 0.6 g (instead of 0.55 g) of DVFH was charged.

A water-insoluble, elastic, crosslinked polymer was produced.

### Example 27

### Synthesis of Branched PVP with 1.0% AVF

In a 1-L resin kettle equipped with an anchor agitator, thermocouple, gas inlet and reflux condenser, 100 g of N-vinyl-2-pyrrolidone, 200 g of water and 1.0 g of N-allyl-N-vinyl formamide (AVF) were charged. The reaction mixture was purged with nitrogen for 30 minutes and then 0.649 g of a 26% NH₄OH aqueous solution was added. With agitation and nitrogen purging, the reactor was heated to 65° C., then 1.79 g of a 35% H₂O₂ aqueous solution was added followed by 0.01 g of a 0.12% CuSO₄ aqueous solution. After 15 minutes of reaction, 0.67 g of a 35% H₂O₂ aqueous solution was charged into the reactor. The reaction was held for 15 minutes, and then 0.55 g of a 35% H₂O₂ aqueous solution was charged into the reactor. After 3 hours, the reaction mixture was cooled to room temperature to discharge the product.

The K-value of a 1% aqueous solution of this polymer was 31.3. M_{w} was 72,480 Da. GPC recovery was 102%, suggesting negligible or zero crosslinked/gel content.

### Example 28

### Synthesis of Branched PVP with 1.0% AVF

Example 27 was substantially repeated, with the exception that 2.5 g (instead of 1.0 g) of AVF was charged.

The K-value of a 1% aqueous solution of this polymer was 46.3. M_{w} was 675,360 Da. GPC recovery was 97%, suggesting negligible or zero crosslinked/gel content. The glass transition temperature was found to be 168.8° C., as determined by differential scanning calorimetry.

### Example 29

### Synthesis of Branched PVP with 1.0% AVF

Example 28 was repeated.

The K-value of a 1% aqueous solution of this polymer was 37.9. M_{w} was 151,380 Da. GPC recovery was 99%, suggesting negligible or zero crosslinked/gel content.

### Example 30

### Synthesis of Branched PVP with 1.0% AVF

Example 28 was repeated.

The K-value of a 1% aqueous solution of this polymer was 39.8. M_{w} was 300,820 Da. GPC recovery was 99%, suggesting negligible or zero crosslinked/gel content.

### Comparative Example 3

### Synthesis of Crosslinked PVP with 3.78% AVF

Example 27 was with the exception that 3.78 g (instead of 1.0 g) of AVF was charged.

A water-insoluble, elastic, crosslinked polymer was produced.

### Example 31

### Synthesis of Branched Poly(95% VP-Co-5% VCL) with 0.55% DVFH Branching Agent (Non-Isothermal Process)

In a 1-L resin kettle equipped with an anchor agitator, thermocouple, gas inlet and reflux condenser, 95 g of N-vinyl-2-pyrrolidone, 5 g N-vinyl-2-caprolactam, 200 g of water and 0.55 g of 1,6-di-(N-vinyl formamido) hexane were charged. The reaction mixture was purged with nitrogen for 30 minutes and then 0.649 g of a 26% NH₄OH aqueous solution was added. With agitation and nitrogen purging, the reactor was heated to 65° C., then 1.79 g of a 35% H₂O₂ aqueous solution were added, followed by 0.01 g of a 0.12% CuSO₄ aqueous solution. After 15 minutes of reaction, 0.67 g of a 35% H₂O₂ aqueous solution was charged into the reactor. The reaction was held for 15 minutes, then 0.55 g of a 35% H₂O₂ aqueous solution was charged into the reactor. The reaction was held at this temperature for 3 hours, and then 0.5 g of tert-amyl peroxy-2-ethylhexanoate (Trigonox® 121) was charged into the reactor. After one hour another 0.5 g of tert-amyl peroxy-2-ethylhexanoate (Trigonox® 121) was charged. Then, after 3 hours, the reaction mixture was cooled to room temperature to discharge the product.

The residual content of N-vinyl-2-pyrrolidone was less than 100 ppm, and the residual content of N-vinyl-2-caprolactam was less than 800 ppm. The K-value of a 1% aqueous solution of this polymer was 31. M_{w} was 76,830 Da, and the GPC recovery was 98%, suggesting negligible or zero crosslinked/gel content. The glass transition temperature was 155.7° C., as determined by differential scanning calorimetry.

### Example 32

### Synthesis of Branched Poly(95% VP-Co-5% VCL) with 0.65% DVFH Branching Agent (Non-Isothermal Process)

Example 31 was substantially repeated, except 0.65 g (instead of 0.55 g) of DVFH was used.

The residual content of N-vinyl-2-pyrrolidone was less than 100 ppm, and the residual content of N-vinyl-2-caprolactam was less than 800 ppm. The K-value of a 1% aqueous solution of this polymer was 38. M_{w} was 244,802 Da, and the GPC recovery was 100%. Complete GPC recovery indicates negligible or zero crosslinked/gel content.

### Example 33

### Synthesis of Poly(95% VP-Co-5% VCL) with 0.75% DVFH Branching Agent (Non-Isothermal Process)

Example 31 was substantially repeated, except 0.75 g (instead of 0.55 g) of DVFH was used.

The residual content of N-vinyl-2-pyrrolidone was less than 100 ppm, and the residual content of N-vinyl-2-caprolactam was less than 800 ppm. The K-value of a 1% aqueous solution of this polymer was 39.7. M_{w} was 320,329 Da, and the GPC recovery was 99%, suggesting negligible or zero crosslinked/gel content.

### Example 34

### Synthesis of Poly(95% VP-Co-5% VCL) with 1% DVFH Branching Agent (Non-Isothermal Process)

In a 1-L resin kettle equipped with an anchor agitator, thermocouple, gas inlet and reflux condenser, 95 g of N-vinyl-2-pyrrolidone, 5 g N-vinyl-2-caprolactam, 200 g of water, and 1 g of 1,6-di-(N-vinyl formamido) hexane were charged. The reaction mixture was purged with nitrogen for 30 minutes and 0.649 g of a 26% NH₄OH aqueous solution was added. With agitation and nitrogen purging, the reactor was heated to 65° C., then 1.79 g of a 35% H₂O₂ aqueous solution was added followed by 0.01 g of a 0.12% CuSO₄ aqueous solution. After 15 minutes of reaction, 0.67 g of a 35% H₂O₂ aqueous solution was charged into the reactor. The reaction was held for 15 minutes, and then 0.55 g of a 35% H₂O₂ aqueous solution was charged into the reactor. The reaction was held for 3 hours, and then a 0.5 g charge of tert-amyl peroxy-2-ethylhexanoate (Trigonox® 121) was added. After one hour, another 0.5 g charge of tert-amyl peroxy-2-ethylhexanoate (Trigonox® 121) was added. After 3 hours, the reaction mixture was cooled to room temperature to discharge the product.

The residual content of N-vinyl-2-pyrrolidone was less than 100 ppm, and the residual content of N-vinyl-2-caprolactam was less than 800 ppm. The K-value of a 1% aqueous solution of this polymer was 42.6. M_{w} was 123,600 Da.

### Example 35

### Synthesis of Poly(95% VP-Co-5% VCL) with 1% DVFH Branching Agent (Non-Isothermal Process)

Example 34 was repeated.

The residual content of N-vinyl-2-pyrrolidone was less than 100 ppm, and the residual content of N-vinyl-2-caprolactam was less than 800 ppm. The K-value of a 1% aqueous solution of this polymer was 40.1. M_{w} was 473,968 Da, Mₙ was 109,500 Da, and the GPC recovery was 101%. Complete GPC recovery indicates negligible or zero crosslinked/gel content.

### Example 36

### Synthesis of Poly(95% VP-Co-5% VCL) with 1% DVFH Branching Agent (Non-Isothermal Process)

Example 35 was repeated.

The residual content of N-vinyl-2-pyrrolidone was less than 100 ppm, and the residual content of N-vinyl-2-caprolactam was less than 800 ppm. The K-value of a 1% aqueous solution of this polymer was 41.1. Mₙ was 128,200 Da.

### Comparative Example 4

### Synthesis of Crosslinked poly(95% VP-Co-5% VCL) with 1.1% DVFH

Example 36 was substantially repeated, with the exception that 1.1 g (instead of 1.0 g) of DVFH was charged

A water-insoluble, elastic, crosslinked polymer was produced.

### Example 37

### Synthesis of Poly(95% VP-Co-5% VCL) with 0.55% DVFH and 0.32% Mercaptoethanol Chain Transfer Agent (Non-Isothermal Process)

In a 1-L resin kettle equipped with an anchor agitator, thermocouple, gas inlet and reflux condenser, 95 g of N-vinyl-2-pyrrolidone, 5 g N-vinyl-2-caprolactam, 200 g of water, 0.31 g of mercaptoethanol, and 1 g of 1,6-di-(N-vinyl formamido) hexane were charged. The reaction mixture was purged with nitrogen for 30 minutes and 0.649 g of a 26% NH₄OH aqueous solution was added. With agitation and nitrogen purging, the reactor was heated to 65° C., then 1.79 g of a 35% H₂O₂ aqueous solution were added, followed by 0.01 g of 0.12% CuSO₄ aqueous solution. After 15 minutes of reaction, 0.67 g of a 35% H₂O₂ aqueous solution was charged into the reactor. The reaction was held for 15 minutes then 0.55 g of a 35% H₂O₂ aqueous solution was charged into the reactor. The reaction was held for 3 hours, and then a charge 0.5 g of tert-amyl peroxy-2-ethylhexanoate (Trigonox® 121) was added. After one hour another 0.5 g charge of tert-amyl peroxy-2-ethylhexanoate (Trigonox® 121) was added. After 3 hours, the reaction mixture was cooled to room temperature to discharge the product.

The residual content of N-vinyl-2-pyrrolidone was less than 100 ppm, and the residual content of N-vinyl-2-caprolactam was less than 800 ppm. The K-value of a 1% aqueous solution of this polymer was 40. M_{w} was 418,841 Da, Mₙ was 98,200 Da, and the GPC recovery was 102%. Complete GPC recovery indicates negligible or zero crosslinked/gel content. The glass transition temperature was 164.1° C., as measured by differential scanning calorimetry.

### Example 38

### Synthesis of Poly(95% VP-Co-5% VCL) with 0.55% DVFH and 0.32% Mercaptoethanol Chain Transfer Agent (Non-Isothermal Process)

Example 37 was repeated.

The residual content of N-vinyl-2-pyrrolidone was less than 100 ppm, and the residual content of N-vinyl-2-caprolactam was less than 800 ppm. The K-value of a 1% aqueous solution of this polymer was 41.1. Mₙ was 114,700 Da.

### Example 39

### Synthesis of Poly(95% VP-Co-5% VCL) with 0.55% DVFH and 0.32% Mercaptoethanol Chain Transfer Agent (Non-Isothermal Process)

Example 37 was repeated.

The residual content of N-vinyl-2-pyrrolidone was less than 100 ppm, and the residual content of N-vinyl-2-caprolactam was less than 800 ppm. The K-value of a 1% aqueous solution of this polymer was 40. Mₙ was 126,400 Da.

### Example 40

### Synthesis of Poly(90% VP-Co-10% VCL) with 1% DVFH (Non-Isothermal Process)

In a 1-L resin kettle equipped with an anchor agitator, thermocouple, gas inlet and reflux condenser, 90 g of N-vinyl-2-pyrrolidone, 10 g N-vinyl-2-caprolactam, 200 g of water and 1 g 1,6-di-(N-vinyl formamido) hexane were charged. The reaction mixture was purged with nitrogen for 30 minutes, and then 0.649 g of a 26% NH₄OH aqueous solution was added. With agitation and nitrogen purging, the reactor was heated to 65° C., then 1.79 g of a 35% H2O2 aqueous solution was added, followed by 0.01 g of a 0.12% CuSO₄ aqueous solution. After 15 minutes of reaction, 0.67 g of a 35% H₂O₂ aqueous solution was charged into the reactor. The reaction was held for 15 minutes, then 0.55 g of a 35% H₂O₂ aqueous solution was charged into the reactor. The reaction was held for 3 hours then 0.5 g charge of tert-amyl peroxy-2-ethylhexanoate (Trigonox® 121) was added. After one hour, another 0.5 g charge tert-amyl peroxy-2-ethylhexanoate (Trigonox® 121) was added. After 3 hours, the reaction mixture was cooled to room temperature to discharge the product.

The residual content of N-vinyl-2-pyrrolidone was less than 100 ppm, and the residual content of N-vinyl-2-caprolactam was less than 800 ppm. The K-value of a 1% aqueous solution of this polymer was 38. M_{w} was 176,100 Da, Mₙ was 53,600 Da, and the GPC recovery was 97%, suggesting negligible or zero crosslinked/gel content.

### Example 41

### Synthesis of Poly(90% VP-Co-10% VCL) with 1% DVFH (Non-Isothermal Process)

Example 40 was repeated.

The residual content of N-vinyl-2-pyrrolidone was less than 100 ppm, and the residual content of N-vinyl-2-caprolactam was less than 800 ppm. The K-value of a 1% aqueous solution of this polymer was 35.5. M_{w} was 450,940 Da, Mₙ was 90,900 Da, and the GPC recovery was 97%, suggesting negligible or zero gel content.

### Example 42

### Synthesis of Poly(90% VP-Co-10% VCL) with 1% DVFH (Non-Isothermal Process)

Example 40 was repeated.

The residual content of N-vinyl-2-pyrrolidone was less than 100 ppm, and the residual content of N-vinyl-2-caprolactam was less than 800 ppm. The K-value of a 1% aqueous solution of this polymer was 35.5. Mₙ was 63,900 Da.

### Example 43

### Synthesis of Poly(90% VP-Co-10% VCL) with 1% DVFH (Non-Isothermal Process)

Example 40 was repeated.

The residual content of N-vinyl-2-pyrrolidone was less than 100 ppm, and the residual content of N-vinyl-2-caprolactam was less than 800 ppm. The K-value of a 1% aqueous solution of this polymer was 37. M_{w} was 296,850 Da, Mₙ was 77,500 Da, and the GPC recovery was 94%, suggesting a low level of crosslinked/gel content.

### Comparative Example 5

### Synthesis of Crosslinked Poly(90% VP-Co-10% VCL) with 1.15% DVFH

Example 43 was substantially repeated, with the exception that 1.15 g (instead of 1.0 g) of DVFH was charged.

A water-insoluble, elastic, crosslinked polymer was produced.

### Example 44

### Synthesis of Poly(90% VP-Co-10% VCL) with 1% DVFH Branching Agent with 0.31% Mercaptoethanol Chain Transfer Agent (Non-Isothermal Process)

In a 1-L resin kettle equipped with an anchor agitator, thermocouple, gas inlet and reflux condenser, 90 g of N-vinyl-2-pyrrolidone, 10 g N-vinyl-2-caprolactam, 200 g of water, 0.31 g of mercaptoethanol, and 1 g of 1,6-di-(N-vinyl formamido) hexane were charged. The reaction mixture was purged with nitrogen for 30 minutes and 0.649 g of a 26% NH₄OH aqueous solution was added. With agitation and nitrogen purging, the reactor was heated to 65° C., then 1.79 g of a 35% H₂O₂ aqueous solution was added, followed by 0.01 g of a 0.12% CuSO₄ aqueous solution. After 15 minutes of reaction, 0.67 g of a 35% H₂O₂ aqueous solution was charged into the reactor. The reaction was held for 15 minutes, and then 0.55 g of a 35% H₂O₂ aqueous solution was charged into the reactor. The reaction was held for 3 hours, and then 0.5 g of tert-amyl peroxy-2-ethylhexanoate (Trigonox® 121) was charged into the reactor. After one hour another 0.5 g of tert-amyl peroxy-2-ethylhexanoate (Trigonox® 121) was charged. Then, after 3 hours, the reaction mixture was cooled to room temperature to discharge the product.

The residual content of N-vinyl-2-pyrrolidone was less than 100 ppm, and the residual content of N-vinyl-2-caprolactam was less than 800 ppm. The K-value of a 1% aqueous solution of this polymer was 40.5. M_{w} was 378,619 Da, Mₙ was 102,500 Da, and the GPC recovery was 101%. Complete GPC recovery indicates negligible or zero crosslinked/gel content. The glass transition temperature was measured by differential scanning calorimetry as 166.6° C.

### Example 45

### Synthesis of Poly(90% VP-Co-10% VCL) with 1% DVFH Branching Agent with 0.31% Mercaptoethanol Chain Transfer Agent (Non-Isothermal Process)

Example 44 was repeated.

The residual content of N-vinyl-2-pyrrolidone was less than 100 ppm, and the residual content of N-vinyl-2-caprolactam was less than 800 ppm. The K-value of a 1% aqueous solution of this polymer was 41.3. M_{w} was 433,430 Da, Mₙ was 107,100 Da, and the GPC recovery was 96%, suggesting a negligible or low crosslinked/gel content.

### Example 46

### Synthesis of Poly(90% VP-Co-10% VCL) with 1% DVFH Branching Agent with 0.31% Mercaptoethanol Chain Transfer Agent (Non-Isothermal Process)

Example 44 was repeated.

The residual content of N-vinyl-2-pyrrolidone was less than 100 ppm, and the residual content of N-vinyl-2-caprolactam was less than 800 ppm. The K-value of a 1% aqueous solution of this polymer was 40. M_{w} was 358,160 Da, Mₙ was 93,400 Da, and the GPC recovery was 95%, suggesting a negligible or low crosslinked/gel content.

### Example 47

### Synthesis of Branched PVCL with 0.45% DVFH Branching Agent (Isothermal Process)

In a 1-L resin kettle equipped with an anchor agitator, thermocouple, gas inlet and reflux condenser, 271.5 g of ethanol and 94 g N-vinyl-2-caprolactam (VCL) were added. With agitation and nitrogen purging, the reactor was heated to 70° C., and 0.4 g of the initiator tert-butyl peroxypivalate (Trigonox® 25-C75) was added. After 30 minutes, 0.4 g of the initiator was charged, as was 90 g of N-vinyl-2-caprolactam, 1.25 g of DVFH, and 45 g of ethanol over a period of half an hour. Afterward, 0.6 g of the initiator was added, and the reaction was maintained for an hour and half. Then, 0.4 g of the initiator was charged and the reaction held for 5 hours. At t=8 hours, the temperature was increased to 75° C. and an addition 0.4 g of the initiator was added, and the reaction held for 4 hours. At this point, an aliquot of the batch was removed to determine N-vinyl-2-caprolactam content, then an additional 0.4 g of the initiator was added, holding the reaction to three hours.

The residual content of N-vinyl-2-caprolactam was less than 1000 ppm. The K-value of a 1% aqueous solution of this polymer was 34. M_{w} was 124,760 Da, Mₙ was 43,021 Da, and the GPC recovery was 87%, suggesting a portion of the polymer contained crosslinked/gel content. The glass transition temperature, as measured by DSC, was 163.3° C.

### Example 48

### Synthesis of Branched PVCL with 0.54% DVFH Branching Agent (Isothermal Process)

Example 47 was substantially repeated, with the exception that 0.54% (instead of 0.45%) of DVFH was charged.

The residual content of N-vinyl-2-caprolactam was less than 1000 ppm. The K-value of a 1% aqueous solution of this polymer was 34.4. M_{w} was 220,940 Da, Mₙ was 59,714 Da, and the GPC recovery was 86%, suggesting a portion of the polymer contained crosslinked/gel content. The glass transition temperature, as measured by DSC, was 164.9° C.

### Example 49

### Synthesis of Branched PVCL with 0.54% DVFH Branching Agent (Isothermal Process)

Example 48 was repeated.

The residual content of N-vinyl-2-caprolactam was less than 1000 ppm. The K-value of a 1% aqueous solution of this polymer was 34.4. M_{w} was 267,940 Da, Mₙ was 59,542 Da, and the GPC recovery was 85%, suggesting a portion of the polymer contained crosslinked/gel content. The glass transition temperature, as measured by DSC, was 168° C.

### Example 50

### Synthesis of Branched PVCL with 0.55% DVFH Branching Agent (Isothermal Process)

Example 47 was substantially repeated, with the exception that 0.55% (instead of 0.45%) of DVFH was charged.

The residual content of N-vinyl-2-caprolactam was less than 1000 ppm. The K-value of a 1% aqueous solution of this polymer was 43. M_{w} was 305,520 Da, Mₙ was 78,338 Da, and the GPC recovery was 87%, suggesting a portion of the polymer contained crosslinked/gel content. The glass transition temperature, as measured by DSC, was 174.3° C.

### Example 51

### Synthesis of Branched PVCL with 0.55% DVFH Branching Agent (Isothermal Process)

Example 50 was repeated.

The residual content of N-vinyl-2-caprolactam was less than 1000 ppm. The K-value of a 1% aqueous solution of this polymer was 43. M_{w} was 401,490 Da, Mₙ was 81,937 Da, and the GPC recovery was 82%, suggesting a portion of the polymer contained crosslinked/gel content.

### Example 52

### Synthesis of Branched PVCL with 0.65% DVFH Branching Agent (Isothermal Process)

Example 47 was substantially repeated, with the exception that 0.65% (instead of 0.45%) of DVFH was charged.

The residual content of N-vinyl-2-caprolactam was less than 1000 ppm. The K-value of a 1% aqueous solution of this polymer was 53. M_{w} was 264,760 Da, Mₙ was 63,038 Da, and the GPC recovery was 77%, suggesting a portion of the polymer contained crosslinked/gel content. The glass transition temperature, as measured by DSC, was 155.6° C.

### Example 53

### Synthesis of Branched PVCL with 0.25% DVFH Branching Agent (Isothermal Process)

Example 47 was substantially repeated, with the exception that the polymerization solvent was a blend of 261.89 g of ethanol, and 105.63 g of water (instead of 271.5 g of ethanol), and 0.89 g (instead of 1.25 g) of DVFH were charged.

The residual content of N-vinyl-2-caprolactam was less than 1000 ppm. The K-value of a 1% aqueous solution of this polymer was 35. M_{w} was 189,500 Da, Mₙ was 70,185 Da, and the GPC recovery was 101%. Complete GPC recovery indicates negligible or zero crosslinked/gel content.

### Example 54

### Synthesis of Branched PVCL with 0.35% DVFH Branching Agent (Isothermal Process)

Example 53 was substantially repeated, with the exception that 0.35% (instead of 0.25%) of DVFH was charged.

The residual content of N-vinyl-2-caprolactam was less than 1000 ppm. The K-value of a 1% aqueous solution of this polymer was 35. M_{w} was 202,200 Da, Mₙ was 69,724 Da, and the GPC recovery was 87%, suggesting a portion of the polymer contained crosslinked/gel content.

### Example 55

### Synthesis of Branched PVCL with 0.45% DVFH Branching Agent (Isothermal Process)

Example 53 was substantially repeated, with the exception that 0.45% (instead of 0.25%) of DVFH was charged.

The residual content of N-vinyl-2-caprolactam was less than 1000 ppm. The K-value of a 1% aqueous solution of this polymer was 37. M_{w} was 240,000 Da, Mₙ was 104,348 Da, and the GPC recovery was 86%, suggesting a portion of the polymer contained crosslinked/gel content. The glass transition temperature, as measured by DSC, was 166° C.

### Example 56

### Synthesis of Branched PVCL with 0.45% DVFH Branching Agent (Isothermal Process)

Example 55 was repeated.

The residual content of N-vinyl-2-caprolactam was less than 1000 ppm. The K-value of a 1% aqueous solution of this polymer was 44. M_{w} was 315,550 Da, Mₙ was 80,910 Da, and the GPC recovery was 83%, suggesting a portion of the polymer contained crosslinked/gel content. The glass transition temperature, as measured by DSC, was 170° C.

### Example 57

### Synthesis of Branched PVCL with 0.45% DVFH Branching Agent (Isothermal Process)

Example 55 was repeated.

The residual content of N-vinyl-2-caprolactam was less than 1000 ppm. The K-value of a 1% aqueous solution of this polymer was 39.1. M_{w} was 365,000 Da, Mₙ was 91,250 Da, and the GPC recovery was 92%, suggesting a portion of the polymer contained crosslinked/gel content. The glass transition temperature, as measured by DSC, was 176° C.

### Example 58

### Synthesis of Branched PVCL with 0.45% DVFH Branching Agent (Isothermal Process)

Example 55 was repeated.

The residual content of N-vinyl-2-caprolactam was less than 1000 ppm. The K-value of a 1% aqueous solution of this polymer was 46.4. M_{w} was 420,000 Da, Mₙ was 110,526 Da, and the GPC recovery was 96%, suggesting a small portion of the polymer contained crosslinked/gel content. The glass transition temperature, as measured by DSC, was 171° C.

### Example 59

### Synthesis of Branched PVP with 0.55% DVFH Branching Agent (Isothermal Process)

In a 1-L resin kettle equipped with an anchor agitator, thermocouple, gas inlet and reflux condenser, 600 g of water and 1.8 g of NH₄OH aqueous solution (26% solution) were added. With agitation and nitrogen purging, the reactor was heated to 80° C., then a feed of 200 g of N-vinyl-2-pyrrolidone and 1.30 g of DVFH were fed over a period of two hours and 0.2 g of a 0.12% CuSO₄ solution was charged. After 30 minutes of starting the feed, 1.2 g of 35% H₂O₂ aqueous solution were charged into the reactor. After 30 minutes of reaction time, six shots of H₂O₂ were charged into the reactor over the period 3 hours at 80° C., and the reaction was held for 4 hours.

The residual content of N-vinyl-2-pyrrolidone was less than 100 ppm. The K-value of a 1% aqueous solution of this polymer was 31.4. M_{w} was 75,917 Da, Mₙ was 18,979 Da, and the GPC recovery was 94%, suggesting a portion of the polymer contained crosslinked/gel content. The glass transition temperature, as measured by DSC, was 139° C.

### Example 60

### Synthesis of Branched PVP with 0.65% DVFH Branching Agent (Isothermal Process)

Example 59 was substantially repeated, with the exception that 0.65% (instead of 0.55%) of DVFH was charged.

The residual content of N-vinyl-2-caprolactam was less than 100 ppm. The K-value of a 1% aqueous solution of this polymer was 43.2. M_{w} was 358,152 Da, Mₙ was 43,677 Da, and the GPC recovery was 102%. Complete GPC recovery indicates negligible or zero crosslinked/gel content. The glass transition temperature, as measured by DSC, was 163° C.

### Example 61

### Synthesis of Branched PVP with 0.75% DVFH Branching Agent (Isothermal Process)

Example 59 was substantially repeated, with the exception that 0.75% (instead of 0.55%) of DVFH was charged.

The residual content of N-vinyl-2-pyrrolidone was less than 100 ppm. The K-value of a 1% aqueous solution of this polymer was 50.8. M_{w} was 837,152 Da, Mₙ was 102,080 Da, and the GPC recovery was 100%. Complete GPC recovery indicates negligible or zero crosslinked/gel content. The glass transition temperature, as measured by DSC, was 166° C.

The polymers and properties for all the above Examples 1-61 and Comparative Examples 1-7 are summarized in Table 2.

TABLE 2
Summary of polymers from Examples 1-60 and Comparative Examples 1-5.

| | | | chain | residual | | | | | | |
|---|---|---|---|---|---|---|---|---|---|---|
| | | branching | transfer | monomer (ppm) | | | | | GPC | |
| exp. | polymer | agent | agent | VP | VCL | K-value | M_{w} | Mₙ | recovery | Tg (° C.) |
| 1 | b-PVP | 0.7% DVFOO | 0 | <100 | — | 37.7 | 229,000 | 39,110 | — | — |
| | 11975-19 | | | | | | | | | |
| 2 | b-PVP | 0.7% DVFOO | 0 | 340 | — | 42.7 | 243,400 | 35,210 | — | — |
| | 11975-7 | | | | | | | | | |
| 3 | b-PVP | 0.7% DVFOO | 0 | 112 | — | 46.1 | 391,410 | 37,181 | — | — |
| | 11975-9 | | | | | | | | | |
| 4 | b-PVP | 0.85% DVFOO | 0 | <100 | — | 42.2 | 372,000 | 46,470 | — | — |
| | 11975-21 | | | | | | | | | |
| 5 | b-PVP | 0.85% DVFOO | 0 | <100 | — | 41.1 | 441,000 | 59,740 | — | — |
| | 11975-23 | | | | | | | | | |
| 6 | b-PVP | 1.0% DVFOO | 0 | <100 | — | 49.3 | 249,000 | 30,490 | — | — |
| | 11975-15 | | | | | | | | | |
| 7 | b-PVP | 1.0% DVFOO | 0 | <60 | — | 43.8 | 508,000 | 63,780 | — | — |
| | 11975-17 | | | | | | | | | |
| 8 | b-PVP | 1.0% DVFOO | 0 | <60 | — | 50.5 | 1,416,000 | 110,600 | — | — |
| | 11884-149 | | | | | | | | | |
| 9 | b-PVP | 1.0% DVFOO | 0 | 428 | — | 52.1 | 681,000 | 87,600 | — | — |
| | 11975-5 | (50%/50%) | | | | | | | | |
| 10 | b-PVP | 1.0% DVFOO | 0 | 271 | — | 43.3 | 828,350 | 78,050 | — | — |
| | 11975-1 | (75%/25%) | | | | | | | | |
| 11 | b-PVP | 1.0% DVFOO | 0 | 130 | — | 41.1 | 450,000 | 57,120 | — | — |
| | 11975-11 | (75%/25%) | | | | | | | | |
| comp. 1 | XL-PVP | 1.25% DVFOO | 0 | | — | — | — | — | — | — |
| 12 | b-PVP | 1.0% DVFOO | 0 | <100 | — | 38.2 | 331,710 | — | 102% | — |
| | 12053-11 | | | | | | | | | |
| 13 | b-PVP | 1.0% DVFOO | 0 | <100 | — | 39.6 | 370,510 | — | 100% | — |
| | 12053-12 | | | | | | | | | |
| 14 | b-PVP | 1.0% DVFOO | 0 | <100 | — | 38.4 | 359,330 | — | 99% | 164.9 |
| | 12053-16 | | | | | | | | | |
| 15 | b-PVP | 1.0% DVFOO | 0.3% MCE | <100 | — | 48.1 | 713,530 | — | 99% | 166.5 |
| | 12053-18 | | | | | | | | | |
| 16 | b-PVP | 1.0% DVFOO | 0.3% MCE | <100 | — | 46.1 | 697,160 | | 98% | 168.2 |
| | 12053-23 | | | | | | | | | |
| 17 | b-PVP | 1.0% DVFOO | 0.3% MCE | <100 | — | 44.4 | 936,460 | — | 97% | — |
| | 12053-24 | | | | | | | | | |
| 18 | b-PVP | 0.50% DVFH | 0 | <100 | — | 40 | 310,000 | — | 99.1% | — |
| 19 | b-PVP | 0.55% DVFH | 0 | <100 | — | 50.5 | 621,550 | — | 100 | 164.8 |
| | 12053-122 | | | | | | | | | |
| 20 | b-PVP | 0.55% DVFH | 0 | <100 | — | 48.5 | 679,950 | — | 101 | — |
| | 12053-111 | | | | | | | | | |
| 21 | b-PVP | 0.55% DVFH | 0 | <100 | — | 48.6 | 649,590 | — | 98 | — |
| | 12053-15 | | | | | | | | | |
| 22 | b-PVP | 0.55% DVFH | 0 | <100 | — | 43.2 | 400,000 | — | — | — |
| | 12053-117 | | | | | | | | | |
| 23 | b-PVP | 0.55% DVFH | 0 | <100 | — | 46 | 877,580 | — | 101% | 165 |
| | 12053-145 | | | | | | | | | |
| 24 | b-PVP | 0.55% DVFH | 0.2% MCE | <100 | — | 41 | 293,200 | 90,600 | 96% | 164.8 |
| | 12053-64 | | | | | | | | | |
| 25 | b-PVP | 0.55% DVFH | 0.2% MCE | <100 | — | 44 | 151,900 | 52,900 | 103% | — |
| | 12053-66 | | | | | | | | | |
| 26 | b-PVP | 0.55% DVFH | 0.2% MCE | <100 | — | 40.5 | 216,910 | 69,800 | 97% | — |
| | 12053-69 | | | | | | | | | |
| comp. 2 | XL-PVP | 0.6% DVFH | 0 | — | — | — | — | — | — | — |
| 27 | b-PVP | 1% AVF | 0 | — | — | 31.3 | 72,480 | — | 102% | — |
| | 12053-20 | | | | | | | | | |
| 28 | b-PVP | 2.50% AVF | 0 | — | — | 46.3 | 675,360 | — | 97% | 168.8 |
| | 12053-25 | | | | | | | | | |
| 29 | b-PVP | 2.50% AVF | 0 | — | — | 37.9 | 151,380 | — | 99% | |
| | 12053-28 | | | | | | | | | |
| 30 | b-PVP | 2.50% AVF | 0 | — | — | 39.8 | 300,820 | — | 99% | |
| | 12053-29 | | | | | | | | | |
| comp. 3 | XL-PVP | 3.78% AVF | 0 | — | — | — | — | — | — | — |
| | 12053-22 | | | | | | | | | |
| 31 | b-P(95 VP-5 | 0.55% DVFH | 0 | <100 | <800 | 31 | 76,830 | — | 98% | 155.7 |
| | VCL) 12053- | | | | | | | | | |
| | 43 | | | | | | | | | |
| 32 | b-P(95 VP-5 | 0.65% DVFH | 0 | <100 | <800 | 38 | 244,802 | — | 100% | — |
| | VCL) 12053- | | | | | | | | | |
| | 50 | | | | | | | | | |
| 33 | b-P(95 VP-5 | 0.75% DVFH | 0 | <100 | <800 | 39.7 | 320,329 | — | 99% | — |
| | VCL) 12053- | | | | | | | | | |
| | 51 | | | | | | | | | |
| 34 | b-P(95 VP-5 | 1.0% DVFH | 0 | <100 | <800 | 42.6 | | 123,600 | — | 159.6 |
| | VCL) 12053- | | | | | | | | | |
| | 52 | | | | | | | | | |
| 35 | b-P(95 VP-5 | 1.0% DVFH | 0 | <100 | <800 | 40.1 | 473,968 | 109,500 | 101% | — |
| | VCL) 12053- | | | | | | | | | |
| | 59 | | | | | | | | | |
| 36 | b-P(95 VP-5 | 1.0% DVFH | 0 | <100 | <800 | 41.1 | — | 128,200 | — | — |
| | VCL) 12053- | | | | | | | | | |
| | 62 | | | | | | | | | |
| comp. 4 | XL-P(95 VP-5 | 1.1% DVFH | 0 | — | — | — | — | — | — | — |
| | VCL) | | | | | | | | | |
| 37 | b-P(95 VP-5 | 0.55% DVFH | 0.32% | <100 | <800 | 40 | 418,841 | 98,200 | 102% | 164.1 |
| | VCL) 12053- | | MCE | | | | | | | |
| | 58 | | | | | | | | | |
| 38 | b-P(95 VP-5 | 0.55% DVFH | 0.32% | <100 | <800 | 41.1 | — | 114,700 | — | — |
| | VCL) 12053- | | MCE | | | | | | | |
| | 60 | | | | | | | | | |
| 39 | b-P(95 VP-5 | 0.55% DVFH | 0.32% | <100 | <800 | 41.2 | — | 126,400 | — | — |
| | VCL) 12053- | | MCE | | | | | | | |
| | 61 | | | | | | | | | |
| 40 | b-P(95 VP-10 | 1.0% DVFH | 0 | <100 | <800 | 38 | 176,100 | 53,600 | 97% | — |
| | VCL) 12053- | | | | | | | | | |
| | 70 | | | | | | | | | |
| 41 | b-P(95 VP-10 | 1.0% DVFH | 0 | <100 | <800 | 35.5 | 450,940 | 90,900 | 100% | — |
| | VCL) 12053- | | | | | | | | | |
| | 74 | | | | | | | | | |
| 42 | b-P(95 VP-10 | 1.0% DVFH | 0 | <100 | <800 | 35.5 | — | 63,900 | — | — |
| | VCL) 12053- | | | | | | | | | |
| | 79 | | | | | | | | | |
| 43 | b-P(95 VP-10 | 1.0% DVFH | 0 | <100 | <800 | 37 | 296,850 | 77,500 | 94% | — |
| | VCL) 12053- | | | | | | | | | |
| | 80 | | | | | | | | | |
| comp. 5 | XL-P(95 VP- | 1.1% DVFH | 0 | — | — | — | — | — | — | — |
| | 10 VCL) | | | | | | | | | |
| 44 | b-P(95 VP-10 | 1.0% DVFH | 0.31% | <100 | <800 | 40.5 | 378,619 | 102,500 | 101% | 166.6 |
| | VCL) 12053- | | MCE | | | | | | | |
| | 77 | | | | | | | | | |
| 45 | b-P(95 VP-10 | 1.0% DVFH | 0.31% | <100 | <800 | 41.3 | 443,430 | 107,100 | 96% | |
| | VCL) 12053- | | MCE | | | | | | | |
| | 81 | | | | | | | | | |
| 46 | b-P(95 VP-10 | 1.0% DVFH | 0.31% | <100 | <800 | 40 | 358,160 | 93,400 | 95% | |
| | VCL) 12053- | | MCE | | | | | | | |
| | 82 | | | | | | | | | |
| 47 | b-PVCL | 0.45% DVFH | 0 | — | <1000 | 34 | 124,760 | 43,020 | 87% | 163.3 |
| | 12053-138 | | | | | | | | | |
| 48 | b-PVCL | 0.54% DVFH | 0 | — | <1000 | 34.4 | 220,940 | 59,714 | 86% | 164.9 |
| | 12053-150 | | | | | | | | | |
| 49 | b-PVCL | 0.54% DVFH | 0 | — | <1000 | 36 | 267,940 | 59,542 | 85% | 168 |
| | 12053-151 | | | | | | | | | |
| 50 | b-PVCL | 0.55% DVFH | 0 | — | <1000 | 43 | 305,520 | 78,338 | 87% | 174.3 |
| | 12053-88 | | | | | | | | | |
| 51 | b-PVCL | 0.55% DVFH | 0 | — | <1000 | 44 | 401,490 | 81,937 | 82% | |
| | 12053-152 | | | | | | | | | |
| 52 | b-PVCL | 0.65% DVFH | 0 | — | <1000 | 53 | 264,760 | 63,038 | 77% | 155.6 |
| | 12053-139 | | | | | | | | | |
| 53 | b-PVCL | 0.25% DVFH | 0 | — | <1000 | 35 | 189,500 | 70,185 | 101% | |
| | 12178-01 | | | | | | | | | |
| 54 | b-PVCL | 0.35% DVFH | 0 | — | <1000 | 35 | 202,200 | 69,724 | 87% | |
| | 12178-02 | | | | | | | | | |
| 55 | b-PVCL | 0.45% DVFH | 0 | — | <1000 | 37 | 240,000 | 104,348 | 86% | 166 |
| | 12178-03 | | | | | | | | | |
| 56 | b-PVCL | 0.45% DVFH | 0 | — | <1000 | 44 | 315,550 | 80,910 | 83% | 170 |
| | 12178-07 | | | | | | | | | |
| 57 | b-PVCL | 0.45% DVFH | 0 | — | <1000 | 39.1 | 365,000 | 91,250 | 92% | 176 |
| | 12178-08 | | | | | | | | | |
| 58 | b-PVCL | 0.45% DVFH | 0 | — | <1000 | 46.4 | 420,000 | 110,526 | 96% | 171 |
| | 12178-09 | | | | | | | | | |
| 59 | b-PVP | 0.55% DVFH | 0 | — | <1000 | 31.4 | 75,917 | 18,979 | 94% | 139 |
| | 12053-131 | | | | | | | | | |
| 60 | b-PVP | 0.65% DVFH | 0 | — | <1000 | 43.2 | 358,152 | 43,677 | 102% | 163 |
| | 12053-132 | | | | | | | | | |
| 61 | b-PVP | 0.75% DVFH | 0 | — | <1000 | 50.8 | 837,060 | 102,080 | 100% | 166 |
| | 12053-134 | | | | | | | | | |

### Example 62

### Mark-Houwink Plots for Branched PVP Polymers

Mark-Houwink plots were constructed using the measured values of the intrinsic viscosity ([η]) and weight-average molecular weight. Polymers studied in this analysis include those made with the DVFOO Branching Agent, both with and without mercaptoethanol as a chain transfer agent. Linear PVP polymers made without any branching agent served as the control.

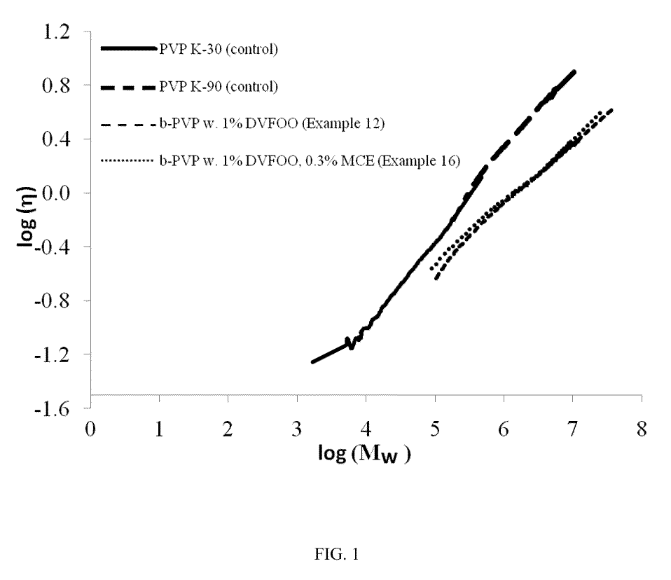

Polymers of the invention exhibit Mark-Houwink plot behavior typical of branched polymers as they do not resemble the characteristics of their linear analogues. Linear PVP K-30 and PVP K-90 in water were found to have a Mark-Houwink exponent of α=0.66 (FIG. 1) (which is independent of linear PVP molecular weight), which conforms to the expected value of about 0.7 for linear homopolymers in a good solvent with a random coil conformation [Costello et al., Polymer, 43, (2002), 245-254]. The invention's polymers, however, are characterized by a lower value of α, of about 0.4. This value is consistent with known behavior of branched polymers [Cheng, et al., Macromol. Rapid Commun., 21, (2000), 846-852]. This result has at least two interpretations: First, at equal molecular weight, polymers of the invention display a lower viscosity than their linear counterparts. This result can be effectively exploited for properties that depend on molecular weight (such as film formation, stiffness, enhanced hair styling durability, hair curl retention, and binding) without encountering viscosity limitations (such as sprayability, pumpability, spreadability).

### Example 63

### Molecular Weight and K-Value Relationships

To extend the understanding of molecular weight provided in Example 1, an analysis was made comparing the weight-average molecular weight and K-value to the amount of added branching agent, DVFOO.

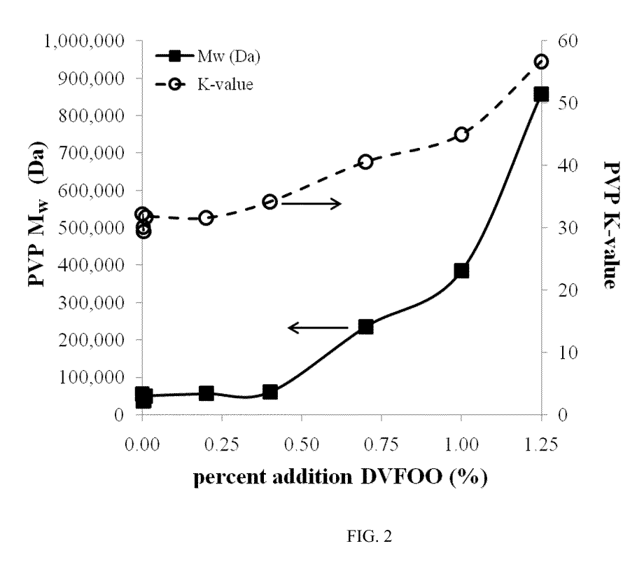

PVP molecular weight increased from about 35,000 Da (without branching agent) to about 60,000 Da with 0.4% DVFOO addition, to about 230,000 Da with 0.7% DVFOO addition, and proceeded to about 860,000 Da with 1.25% DVFOO addition (FIG. 2). Concurrently, the K-value of the branched PVP polymers also increased, and ranged from 30 (0% DVFOO) to 56 (1.25% DVFOO). (Above 1.25% DVFOO addition, the branched PVP began to resemble a hydrogel in water.)

At equal molecular weight, polymers of the invention possess lower K-value than their linear PVP counterparts. Linear PVP K-60 polymer sold into commercial trade by International Specialty Products has a weight-average molecular weight ranging from 240,000 Da-450,000 Da [PVP (Polyvinyl pyrrolidone), marketing brochure, ISP]. For this same range of weight-average molecular weight, FIG. 2 shows branched PVP polymers of the present invention have a lower K-value, from about 34 to 45.

### Example 64

### Effect of DVFOO Addition Level on Mᵥ and K-Value

Molecular weight distributions of the present invention's polymers were evaluated using gel permeation chromatography.

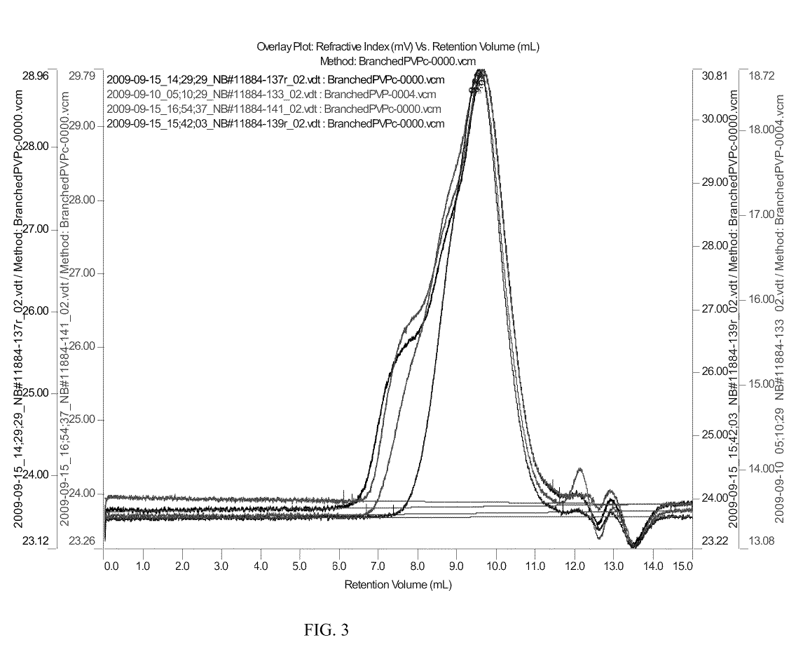

Compared to the linear PVP control polymers, the invention's branched polymers exhibited a broader distribution in molecular weight (FIG. 3). Contained within the branched polymers was a high molecular weight component that eluted before the lower molecular weight fractions.

### Example 65

### Sprayability of the Invention's Branched Polymers in VOC Formula #1

Six polymers of the invention were evaluated for their sprayability in a formula containing volatile organic carbon (VOC). The tested formulas contained either 3% or 5% (w/w total) of polymers of the invention, 55% ethanol, and the balance was a sufficient quantity of water. The polymers represented branched PVP (made with DVFOO, DVFH, AVF), and branched poly(VP-co-VCL) (made with DVFH). Each formula was loaded into an Optima spray pump (Aptar), which is designed to deliver fine mists with a 140 μL output. The droplet size distribution from each formula was measured using a Malvern SprayTec RTS 5214. Formula (and therefore polymer) sprayability was assigned when the volume-average droplet diameter (Dv₅₀) from the Optima spray pump was less than 100 μm.

Each tested polymer attained sprayability at the 3% (w/w total) addition level (Table 3). Five of the eight polymers also were sprayable at the 5% (w/w total) addition level.

TABLE 3
Results from the sprayability test of Example 65

| polymer | | | | | sprayability attained | |
|---|---|---|---|---|---|---|
| of | | branching | | | 3% (w/w | 5% (w/w |
| Example | polymer | agent | | M_{w} (Da) | total) | total) |
| 8 | b-PVP | 1% | DVFOO | 1,416,000 | yes | no |
| 19 | b-PVP | 0.55% | DVFH | 621,550 | yes | no |
| 30 | b-PVP | 2.50% | AVF | 300,820 | yes | yes |
| 37 | b-95% PVP/5% VCL | 0.55% | DVFH | 418,841 | yes | yes |
| 35 | b-95% PVP/5% VCL | 1.0% | DVFH | 473,968 | yes | yes |
| 25 | b-PVP | 0.55% | DVFH | 151,900 | yes | yes |
| 41 | b-95% PVP/5% VCL | 1.0% | DVFH | 450,940 | yes | yes |
| 44 | b-95% PVP/5% VCL | 1.0% | DVFH | 378,619 | yes | no |

### Example 66

### Droplet Size Distribution of Branched PVP at 3% and 5% Addition Level in VOC Formula #1

The spray patterns and droplet size distributions were measured for four polymers in the VOC formula #1. In addition to measuring the droplet size distribution, the following parameters also were evaluated: formula Brookfield viscosity, formula appearance, spray pattern, and dried film appearance from the spray. Spray pattern was assessed by spraying the formula for 1 second at a vertically-positioned graph paper target located 8″ away, and then immediately outlining the pattern of regular, irregular, and stray wet areas. The spray pattern test was conducted away from interfering drafts. The tested formulas were a subset of those from Example 65; these formulas contained 3% (w/w) or 5% (w/w) polymer, 55% (w/w) ethanol and quantity-sufficient water. The polymers were linear PVP K-30, K-60, and K-90, and the branch PVP of Example 23 (12053-145) (Table 4). Each formula was evaluated in the same pump mechanism as Example 65.

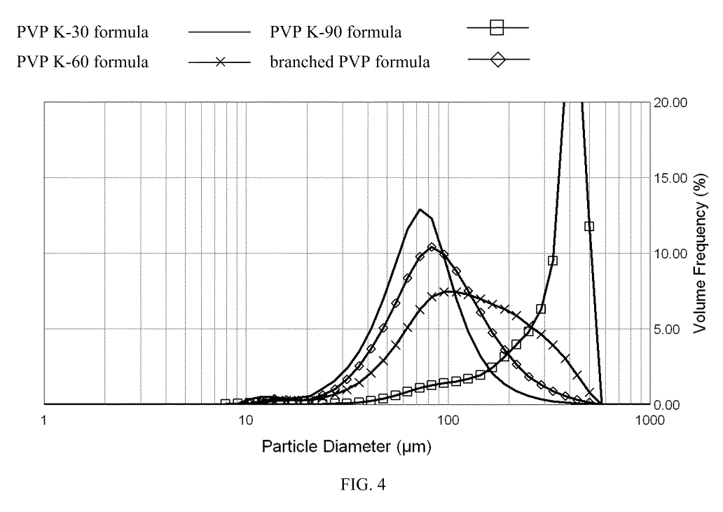

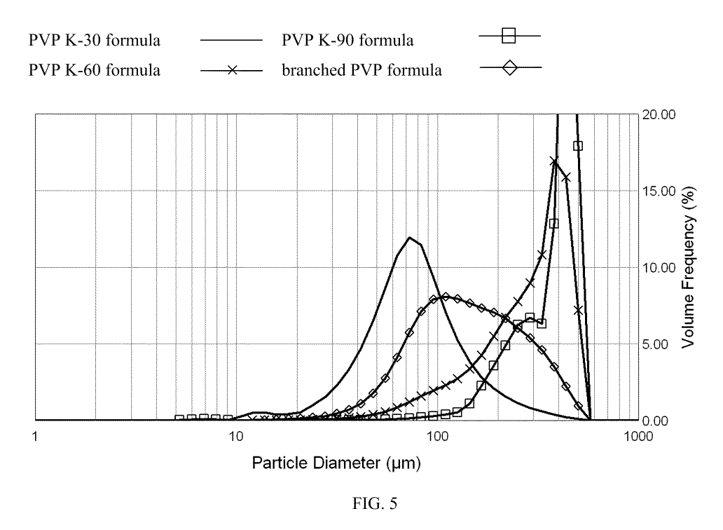

While the branched PVP had a weight-average molecular weight between linear PVP K-69 and K-90, its solution and spray properties was intermediate between linear PVP K-30 and K-60 (Table 5) at both 3% addition level (FIG. 4) and 5% addition level (FIG. 5).

TABLE 4
Droplet size distributions at 3% (w/w)
polymer addition level for Example 66

| | | | | b-PVP test |
|---|---|---|---|---|
| | control formulas | | | formula |
| Polymer Properties: | | | | |
| ID | PVP K-30 | PVP K-60 | PVP K-90 | Example 23 |
| | | | | (12053-145) |
| mean M_{w} (Da) | 58,000 | 400,000 | 1,300,000 | 877,580 |
| Solution Properties: | | | | |
| Brookfield visc. (cP) | 4.4 | 10.3 | 28.4 | 8.7 |
| (test rpm) | (12) | (12) | (6) | (12) |
| solution appearance | clear | clear | clear | clear |
| mist diameter | 4.25″ | 4″ | 1″ | 4″ |
| (@10″ distance | | | | |
| droplet size | | | | |
| distribution | | | | |
| Dv₁₀ (μm) | 34 | 48 | 129 | 40 |
| Dv₅₀ (μm) | 67 | 116 | 350 | 82 |
| Dv₉₀ (μm) | 121 | 287 | 446 | 175 |
| span | 1.31 | 2.06 | 0.91 | 1.65 |
| Dried Film | | | | |
| Properties: | | | | |
| dried film appearance | clear | clear | clear | clear |

TABLE 5
Droplet size distribution at 5% (w/w)
polymer addition level for Example 66

| | | | | b-PVP test |
|---|---|---|---|---|
| | control formulas | | | formula |
| Polymer Properties: | | | | |
| ID | PVP K-30 | PVP K-60 | PVP K-90 | Example 23 |
| | | | | (12053-145) |
| mean M_{w} (Da) | 58,000 | 400,000 | 1,300,000 | 877,580 |
| Solution Properties: | | | | |
| Brookfield visc. (cP) | 6.1 | 20 | 77.4 | 15.1 |
| (test rpm) | (12) | (12) | (3) | (12) |
| solution appearance | clear | clear | clear | clear |
| mist diameter | 4.25″ | 2.5″ | 0.75 | 4″ |
| (@ 10″ distance) | | | | |
| droplet size | | | | |
| distribution | | | | |
| Dv₁₀ (μm) | 35 | 111 | 195 | 60 |
| Dv₅₀ (μm) | 70 | 292 | 386 | 129 |
| Dv₉₀ (μm) | 143 | 426 | 465 | 300 |
| span | 1.55 | 1.08 | 0.7 | 1.86 |
| Dried Film | | | | |
| Properties: | | | | |
| dried film appearance | clear | clear | clear | clear |

### Example 67

### Polymer Reduction of Linear PVP Formulas to Match Branched PVP Droplet Size Distribution

The polymer addition levels for the VOC formula #1 having linear PVP K-60 and K-90 from Example (above) were reduced in order to attain droplet size distributions that matched the measured values for the branched PVP formula at 3% (w/w) polymer level. All formulas contained 55% ethanol, and a quantity-sufficient of water to make the dilutions.

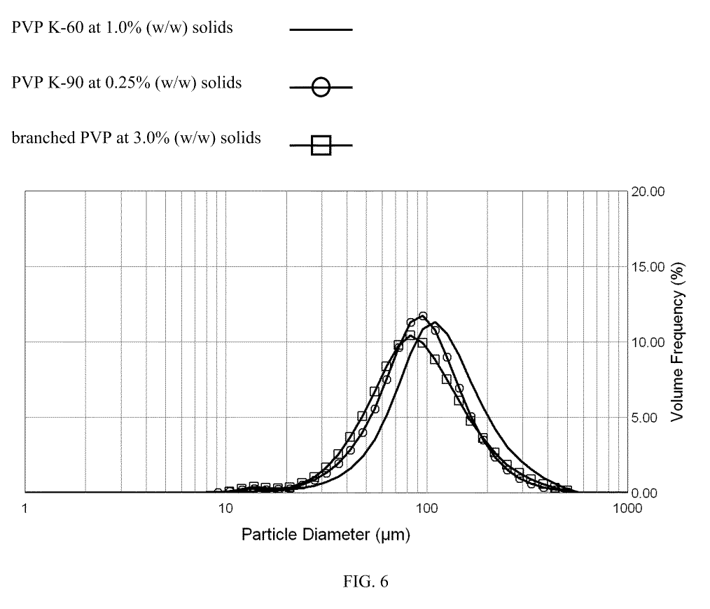

Results from the Malvern SprayTec RTS 5214 showed that equivalent droplet spray diameters were attained by diluting the linear PVP K-60 formula from 3.0% (w/w) to 1.0% (w/w) polymer, and the linear PVP K-90 formula from 3.0% (w/w) to 0.25% (w/w) polymer (Table 6, FIG. 6).

TABLE 6
Droplet size distributions for the formulas of Example 67

| | | | b-PVP test |
|---|---|---|---|
| | control formulas | | formula |
| Polymer Properties: | | | |
| ID | PVP K-60 | PVP K-90 | Example 23 |
| | | | (12053-145) |
| polymer level (w/w) | 1.0% | 0.25% | 3.0% |
| mean M_{w} (Da) | 400,000 | 1,300,000 | 877,580 |
| droplet size distribution: | | | |
| Dv₁₀ (μm) | 53 | 43 | 40 |
| Dv₅₀ (μm) | 104 | 86 | 82 |
| Dv₉₀ (μm) | 210 | 164 | 175 |
| span | 1.5 | 1.4 | 1.65 |

### Example 68

### Sprayability of the Invention's Polymers in VOC Formula #2

Three polymers of the invention were evaluated for their sprayability in a second VOC formula. Like the formula in Example 65, this formula contained 55% VOC, but for this example contained a blend of 35% dimethyl ether and 20% ethanol. The polymers were tested at the 3% (w/w total) addition level. In addition to measuring the droplet size distribution, the following parameters also were evaluated: formula Brookfield viscosity, formula appearance, spray pattern, and dried film appearance from the spray. Spray pattern was assessed by spraying the formula for 1 second at a vertically-positioned graph paper target located 8″ away, and then immediately outlining the pattern of regular, irregular, and stray wet areas. The spray pattern test was conducted away from interfering drafts. Three linear PVP polymers, PVP K-30, PVP K-60, and PVP K-90, in the same formula served as the controls.

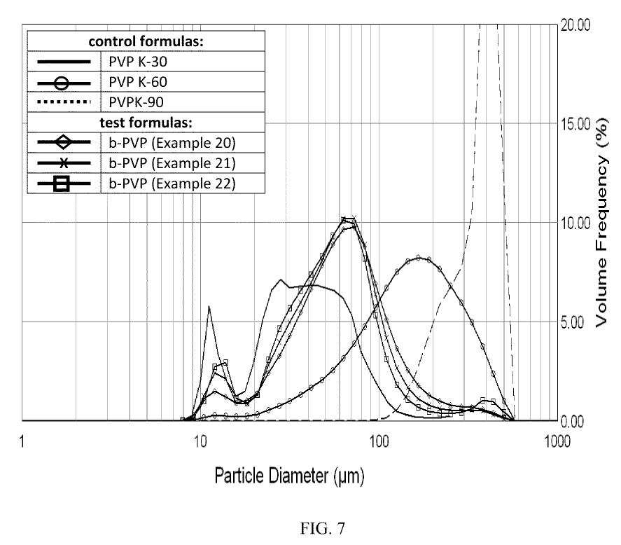

All polymers proved to be sprayable in VOC formula #2. The branched PVP polymers of the present invention had higher mean M_{w} than PVP K-60 and K-90, but exhibited lower solution viscosity (Table 7). Like the control formulas, the test formulas provided clear solutions with excellent mist spray diameters. Advantageously, the branched PVP formulas did not show wet centers. This benefit is attributed, in part, to the smaller droplet sizes. The PVP K-60 and K-90 formulas gave coarse sprays with a Dv₅₀ greater than 100 μm (FIG. 7), whereas mists of the invention's polymer formulas provided a Dv₅₀ less than 100 μm.

TABLE 7
Formula and spray properties for VOC formulas #2 of Example 68.

| | control formulas | | | b-PVP test formulas | | |
|---|---|---|---|---|---|---|
| Polymer Properties: | | | | | | |
| ID | PVP K-30 | PVP K-60 | PVP K-90 | Example 20 | Example 21 | Example 22 |
| | | | | (12053-111) | (12053-115) | 12053-117 |
| mean M_{w} (Da) | 58,000 | 400,000 | 1,300,000 | 679,950 | 649,590 | 400,000 |
| Solution Properties: | | | | | | |
| Brookfield visc. (cP) | 5.1 | 44.5 | 62.8 | 18.1 | 17.1 | 16.0 |
| (test rpm) | (20) | (10) | (6) | (12) | (12) | (12) |
| solution appearance | clear | clear | clear | clear | clear | clear |
| mist diameter | 3.5″ | 3″ | 2.5″ | 2.75″ | 3″ | 3″ |
| (@ 10″ distance) | | wet center | wet center | | | |
| droplet size | | | | | | |
| distribution | | | | | | |
| Dv₁₀ (μm) | 12 | 51 | 195 | 24.15 | 22.4 | 20.51 |
| Dv₅₀ (μm) | 35 | 142 | 352 | 57.35 | 54.7 | 50.99 |
| Dv₉₀ (μm) | 71 | 306 | 445 | 123.74 | 109.56 | 100.91 |
| span | 1.7 | 1.8 | 0.7 | 1.74 | 1.59 | 1.58 |
| Dried Film | | | | | | |
| Properties: | | | | | | |
| dried film | clear | clear | clear | clear | clear | clear |
| appearance | | | | | | |

### Example 69

### Sprayability of the Invention's Branched Polymers in a Reduced VOC Formula

The branched PVP formula of Example 68 (the formula containing the polymer from Example 21 and 20% ethanol) was compared to a formula having the same polymer and polymer level (i.e., 3%), but made in a blend of 35% dimethyl ether, 10% ethanol, and 52% water. (This formula is identical to the formula in Example 64 except it contains more water.)

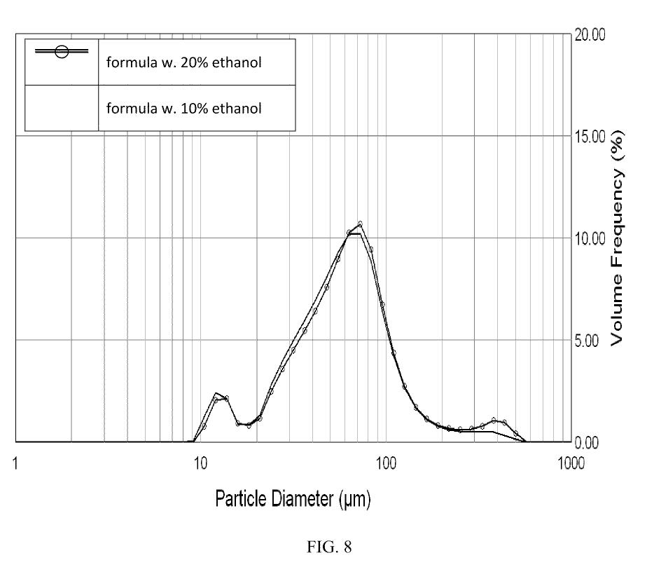

In this environmentally-improved formula, the branched PVP spray performance was not diminished. While this formula exhibited a lower viscosity than the formula with more ethanol, the spray characteristics were essentially unchanged (Table 8) and the droplet size distribution was the same (FIG. 8).

TABLE 8
Formula and spray properties of a reduced
VOC formula of Example 69.

| | b-PVP test formulas | |
|---|---|---|
| Formula | of Example 68 | of Example 69 |
| Polymer Properties: | w. 20% ethanol | w. 10% ethanol |
| ID | Example 21 | Example 21 |
| | (12053-115) | (12053-115) |
| mean M_{w} (Da) | 649,590 | 649,590 |
| Solution Properties: | | |
| Brookfield visc. (cP) | 17.1 | 13.6 |
| (test rpm) | (12) | 12 |
| solution appearance | clear | clear |
| mist diameter | 3″ | 3″ |
| (@ 10″ distance) | | |
| droplet size distribution | | |
| Dv₁₀ (μm) | 22.4 | 23.78 |
| Dv₅₀ (μm) | 54.7 | 57.53 |
| Dv₉₀ (μm) | 109.56 | 117.50 |
| span | 1.59 | 1.63 |
| Dried Film Properties: | | |
| dried film appearance | clear | clear |

### Example 70

### Evaluation of Branched PVP with Thickeners

Six hair gel prototypes were prepared having one of three thickeners, one of two film formers, a base neutralizer, and a preservative (Table 9). The three control formulas (#1 through #3) contained 0.50% of PVP K-90, and the test formulas contained 0.50% branched PVP of Example 8 instead of PVP K-90. The viscosity of each blend was measured using a Brookfield DV II viscometer with spindle 6 operating at 10 rpm for 1 minute at room temperature (25° C.).

Although the branched PVP had a higher molecular weight than the PVP K-90 (1,416,000 Da vs. 1,300,000 Da), the test formulas (#4 through #6) had a lower Brookfield viscosity than the control formulas (#1 through #3) (Table 9).

TABLE 9
Formulas of the polymer blends of Example 70

| | control formulas | | | test formulas | | |
|---|---|---|---|---|---|---|
| ingredient | 1 | 2 | 3 | 4 | 5 | 6 |
| deionized water | 97.05 | 98.26 | 98.20 | 97.05 | 98.26 | 98.20 |
| thickener: | | | | | | |
| arbomer 980 (Carbopol ® | 0.50 | | | 0.50 | | |
| 980, Lubrizol) | | | | | | |
| crylic acid/VP crosspolymer | | 0.50 | | | 0.50 | |
| (UltraThix ™ P-100, ISP) | | | | | | |
| VM/MA decadiene crosspolymer | | | 0.50 | | | 0.50 |
| (Stabilize ® QM, ISP) | | | | | | |
| film former: | | | | | | |
| VP K-90 (ISP) | 0.50 | 0.50 | 0.50 | | | |
| ranched PVP (Example 8) | | | | 0.50 | 0.50 | 0.50 |
| base neutralizer: | | | | | | |
| MP Ultra PC 2000 (Dow) | 0.45 | 0.24 | 0.30 | 0.45 | 0.24 | 0.30 |
| preservative: | | | | | | |
| iquid Germall Plus ® (ISP) | 0.50 | 0.50 | 0.50 | 0.50 | 0.50 | 0.50 |
| total | 100% | 100% | 100% | 100% | 100% | 100% |
| viscosity (cP) | 98,000 | 84,200 | 93,200 | 89,000 | 72,200 | 81,300 |

### Example 71

### Visual Evaluation of the Spray Pattern of Hair Gel Prototypes

Each formula of Example 70 was loaded into a pump system (Precision, P1, 0.18 mL dosage, 0.012″ BMU, Jumbo Dip tube) and then sprayed onto a vertical sheet of graph paper in order to characterize the spray pattern.

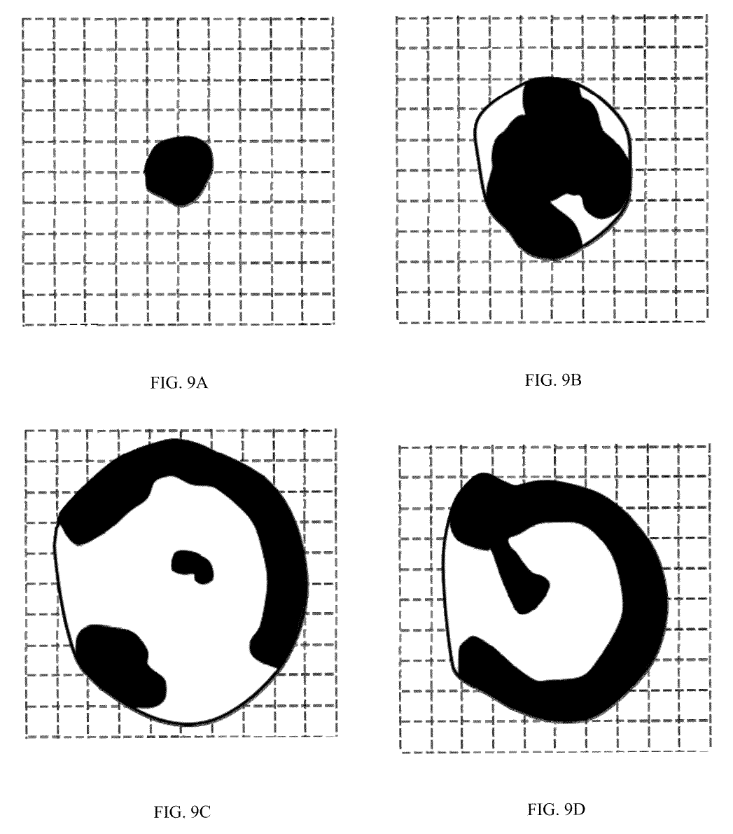

The three control formulas (#1 through #3) with PVP K-90 atomized poorly, and produced narrow-diameter sprays with wet centers (FIG. 9A). The three test formulas containing the branched PVP (#4 through #6) atomized well, and created sprays with diameters up to 2.5-times that of the controls. FIG. 9B is the spray for formula #4, FIG. 9C is the spray for formula #5, and FIG. 9D is the spray pattern for formula #6. In these figures the solid black areas represent the heaviest spray, the white area represent the full spray area, and the dashed lines represent the grid paper.

### Example 72

### High Humidity Curl Retention for the Test Hair Gel Formulas

The three test hair gels of Example 70 (#4 through #6) were applied to hair tresses, and a curling iron was used to impart eight curls for each test formula/tress. After drying, the tresses were placed in a controlled temperature and humidity chamber (80° F., 90% RH) for four hours. Then, the curl retention was measured and the values averaged for each formula.

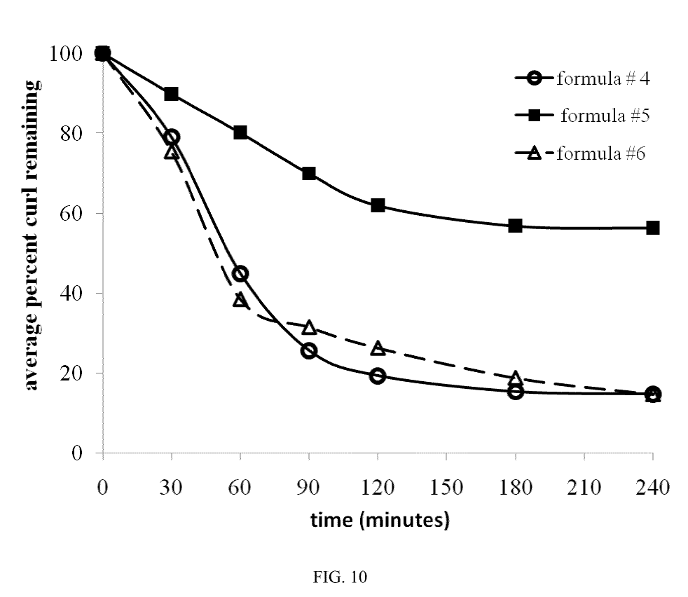

Under these test conditions formula #4 provided about 60% curl retention (FIG. 10). Less curl retention was provided by formulas #5 and #6.

### Example 73

### Hair Styling Gel Formulas

Twelve hair gels were made having the branched PVP of Example 23. These formulas are presented in Table 10, and were prepared as described herein:

In formulas 1-4, Phase A was made by slowly adding a carbomer (Carbopol® 980, The Lubrizol Corporation) to the deionized water and blended for at least one hour using a mixer fitted with a propeller head to achieve polymer hydration. Then, after substituting a paddle head for the propeller mixer head, 2-amino-2-methyl-1-propanol neutralizing amine (AMP™ Ultra PC 2000, Dow Chemical Co.) was added to the blend, and mixing at a moderate setting to avoid bubble formation. Separately, Phase B was prepared by blending the ingredients listed, making use to add the polymer to water first, and then adding the preservative (Liquid Germall® Plus) after a clear mixture was obtained. This Phase B was added to Phase A and blended with moderate mixing to ensure homogeneity.

In formulas 5-8, Phase A was prepared by adding the 0.05% of the -amino-2-methyl-1-propanol neutralizing amine (AMP™ Ultra PC 2000, Dow Chemical Co.) to the deionized water and mixing well using a propeller mixing head. Then, the acrylic acid/VP crosspolymer (UltraThix™ P-100, ISP) was sprinkled-in, and mixing resumed for at least 1 hour. Once the crosspolymer was confirmed to be well-hydrated and the preparation devoid of fish eyes, the remaining amount of the neutralizing amine was added. The propeller mixing head was replaced with a paddle head, and phase moderately mixed to avoid bubble formation. Separately, Phase B was prepared, adding the polymer to water first and then adding the preservative (Liquid Germall® Plus) after a clear mixture was obtained. This Phase B was added to Phase A and blended with moderate mixing to ensure homogeneity.

In formulas 9-12, the PVM/MA decadiene crosspolymer (Stabileze® QM, ISP) solution was added to the deionized water and mixed for at least 15 minutes using a propeller mixing head. Then, the mixing head was replaced with a paddle head, and the neutralizing amine was added blended-in using moderate mixing to avoid air bubble formation. Separately, Phase B was prepared, adding the polymer to water first and then adding the preservative (Liquid Germall® Plus) after a clear mixture was obtained. This Phase B was added to Phase A and blended with moderate mixing to ensure homogeneity.

TABLE 10
The hair styling gel formulas of Example 73

| | addition level (% w/w) | | | | | | | | | | | |
|---|---|---|---|---|---|---|---|---|---|---|---|---|
| ingredient | 1 | 2 | 3 | 4 | 5 | 6 | 7 | 8 | 9 | 10 | 11 | 12 |
| Phase A | | | | | | | | | | | | |
| deionized water | 93.05 | 93.05 | 92.50 | 93.05 | 93.26 | 93.26 | 92.71 | 93.26 | 68.70 | 68.70 | 68.15 | 68.70 |
| Carbopol ® 980 | 0.5 | 0.5 | 0.5 | 0.5 | | | | | | | | |
| UltraThix P-100 | | | | | 0.5 | 0.5 | 0.5 | 0.5 | | | | |
| Stabileze QM | | | | | | | | | 25 | 25 | 25 | 25 |
| (2% sol.) | | | | | | | | | | | | |
| AMP-PC 2000 | 0.45 | 0.45 | 0.45 | 0.45 | 0.24 | 0.24 | 0.24 | 0.24 | 0.3 | 0.3 | 0.3 | 0.3 |
| Phase B | | | | | | | | | | | | |
| deionized water | 5.0 | 5.0 | 5.0 | 5.0 | 5.0 | 5.0 | 5.0 | 5.0 | 5.0 | 5.0 | 5.0 | 5.0 |
| polymer of | 0.5 | | | | 0.5 | | | | 0.5 | | | |
| Example 23 | | | | | | | | | | | | |
| PVP K-90 | | 0.5 | | | | 0.5 | | | | 0.5 | | |
| PVP K-60 | | | 1.05 | | | | 1.05 | | | | 1.05 | |
| PVP K-30 | | | | 0.5 | | | | 0.5 | | | | 0.5 |
| Liquid | 0.5 | 0.5 | 0.5 | 0.5 | 0.5 | 0.5 | 0.5 | 0.5 | 0.5 | 0.5 | 0.5 | 0.5 |
| Germall ® Plus | | | | | | | | | | | | |
| Total | 100 | 100 | 100 | 100 | 100 | 100 | 100 | 100 | 100 | 100 | 100 | 100 |

### Example 74

### Sprayability of the Hair Styling Gel Formulas

The twelve formulas of Example 73 were evaluated for their sprayability using the method described in Example 66. In this example the Brookfield viscosity was measured after 1 minute using spindle #7 operating at 10 rpm. The reported the spray pattern diameter and shape represent the average value from three measurements.

The branched PVP (b-PVP) formulas resembled the PVP K-90 formulas with regard to Brookfield viscosity, but sprayed like the lower molecular weight PVP K-30 formulas (Table 11). In every formula the branched PVP formulas attained the largest average spray pattern diameter.

TABLE 11
Sprayability parameters for the hair
styling gels tested in Example 74

| | | | average | |
|---|---|---|---|---|
| | | | spray | |
| | | | pattern | spray |
| Formula of | Brookfield | | diameter | pattern |
| Example 73 | viscosity (cP) | pH | (cm) | shape |
| Carbopol ® 980 series | | | | |
| #1 (b-PVP) | 79,600 | 6.74 | 7.0 | circular |
| #2 (PVP K-90) | 93,000 | 6.62 | 2.3 | circular |
| #3 (PVP K-60) | 87,200 | 6.98 | 3.2 | circular |
| #4 (PVP K-30) | 83,500 | 7.03 | 7.2 | circular |
| Ultrathix P-100 series | | | | |
| #5 (b-PVP) | 39,900 | 6.82 | 9.2 | circular |
| #6 (PVP K-90) | 46,100 | 6.91 | 2.0 | circular |
| #7 (PVP K-60) | 45,500 | 6.95 | 3.5 | circular |
| #8 (PVP K-30) | 43,500 | 6.94 | 7.3 | circular |
| Stabilize QM series | | | | |
| #9 (b-PVP) | 85,800 | 6.55 | 8.8 | circular |
| #10 (PVP K-90) | 98,000 | 7.2 | 1.5 | circular |
| #11 (PVP K-60) | 91,500 | 7.36 | 3.2 | circular |
| #12 (PVP K-30) | 84,800 | 6.92 | 7.5 | circular |

### Example 75

### Sprayability of the Hair Styling Gel Formulas

In addition to sprayability, the hair styling gel formulas of Example 73 also were tested for their tendencies to flake after combing. The procedure followed for this test was as follows: First, 1.0 g of each formula was applied to European hair tresses that were still wet after washing with a non-care/non-conditioning shampoo. Following styling gel application, each tress was blow dried straight without the use of any styling tools (like a brush or comb). Then, the tresses were supplied to a professional panel that was trained how to comb the tresses (10 combing strokes each) and evaluate for formula flaking on the hair and comb surfaces. A results were ranked using a qualitative scale than ranged from 0 to 10 (Table 12).

All hair styling gels of the invention (having branched PVP) produced significantly less flaking compared to the control formulas having linear PVP K-30, K-60, or K-90 (Table 13). The superior performance was significant at the 95% confidence level for all comparisons, except when comparing formula #5 to #7, where the difference is significant at the 90% confidence level.

TABLE 12
The ranking scale for hair styling flaking

| value | observation |
|---|---|
| 10 | No residue (nothing visible) |
| 9 | Very, very slightly visible |
| 8 | Very slightly visible |
| 7 | Slightly visible |
| 6 | Visible-powdery |
| 5 | Visible-flakes |
| 4 | Considerable |
| 3 | Considerable to heavy |
| 2 | Heavy |
| 1 | Extremely heavy (Hair is completely covered.) |

TABLE 13
Flaking results of Example 75

| Formula of | average flaking score |
|---|---|
| Example 73 | (mean ± 95% CI) |
| Carbopol ® 980 series | |
| #1 (b-PVP) | 4.00 ± 0.57 |
| #2 (PVP K-90) | 2.17 ± 0.86 |
| #3 (PVP K-60) | 2.33 ± 0.33 |
| #4 (PVP K-30) | 2.67 ± 0.33 |
| Ultrathix P-100 series | |
| #5 (b-PVP) | 5.50 ± 0.57 |
| #6 (PVP K-90) | 3.33 ± 1.18 |
| #7 (PVP K-60) | 4.33 ± 1.18 |
| #8 (PVP K-30) | 4.33 ± 0.33 |
| Stabilize QM series | |
| #9 (b-PVP) | 3.83 ± 0.33 |
| #10 (PVP K-90) | 2.17 ± 0.33 |
| #11 (PVP K-60) | 2.00 ± 0.57 |
| #12 (PVP K-30) | 2.67 ± 0.65 |

What is claimed is:

1. A branched polymer resulting from the polymerization of
at least one: (A1) reactive monomer having a vinyl functional group, or (A2) a hybrid reactive monomer having (a) at least one vinyl functional group and (b) at least one reactive non-vinyl functional group, and
at least one: (B1) branching agent having at least two N-vinyl formamide functional groups or (B2) a hybrid branching agent having (a) at least one N-vinyl formamide functional group, and (b) at least one vinyl functional group.

2. The branched polymer according to claim 1 that is at least 10% (w/w) soluble in a solvent for which the corresponding polymer of equal molecular weight made without said (B1) or (B2) also is soluble.

3. The branched polymer according to claim 1 having at least one of: a lower viscosity, enhanced processability, or improved sprayability compared to a corresponding polymer of equal molecular weight synthesized without said (B1) or said (B2).

4. The branched polymer according to claim 1, wherein said (A1) is selected from the group consisting of: maleic anhydrides, vinyl amides, (meth)acrylates, (meth)acrylamides, styrenes, maleimides, maleates, fumarates, cinnamyls, vinyl imidazoles, vinyl pyridines, vinyl acetates, vinyl sulfones, vinyl carbonates, vinyl silanes, allyls, vinyl ethers, and combinations thereof.

5. The branched polymer according to claim 4 wherein said vinyl amide is selected from the group consisting of: N-vinyl-2-pyrrolidone, N-vinyl-2-caprolactam, and combinations thereof.

6. The branched polymer according to claim 1, wherein said wherein said “reactive non-vinyl functional group” in said (A2) is selected from the group consisting of: epoxides, oxetanes, benzoxazines, oxazolines, and combinations thereof.

7. The branched polymer according to claim 1, wherein said (B1) is selected from the group consisting of: 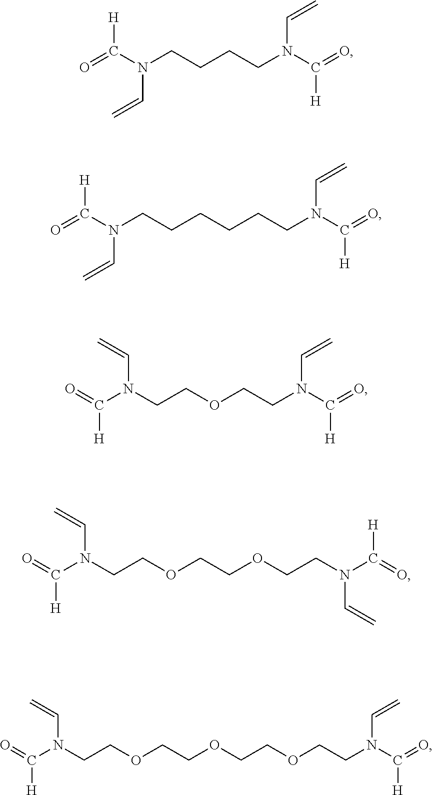
and combinations thereof.

8. The branched polymer according to claim 1 wherein said (B2) is selected from the group consisting of: is selected from the group consisting of: 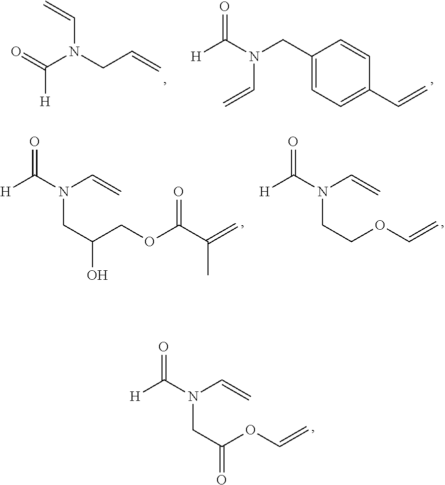
and combinations thereof.

9. The branched polymer according to claim 1 having a weight-average molecular weight from about 200 Da to about 20,000,000 Da.

10. The branched polymer according to claim 1 wherein said (A1) is selected from the group consisting of: N-vinyl-2-pyrrolidone, N-vinyl-2-caprolactam, and combinations thereof; and said (B1) is selected from the group consisting of: 1,8-di-(N-vinyl formamido)-3,6-dioxyoctane, 1,6-di-(N-vinyl formamido) hexane, and combinations thereof.

11. The branched polymer according to claim 1 wherein said (A1) is selected from the group consisting of: N-vinyl-2-pyrrolidone, N-vinyl-2-caprolactam, and combinations thereof; and said (B2) is selected from the group consisting of: N-allyl-N-vinyl formamide.

12. A composition comprising a branched polymer of any one of claim 1, wherein said composition is selected from the group consisting of: adhesive, aerosol, agricultural, beverage, cleaning, coating, detergent, drug, encapsulation, food, lithographic, membrane, oilfield, personal care composition, pharmaceutical/nutritional, and pigment dispersion compositions.

13. The composition according to claim 12 that provides a median volume-average droplet diameter (Dv₅₀) of 130 μm or less.

14. The composition according to claim 13 that provides a circular spray pattern.

15. The personal care composition according to claim 12 that is a hair care, hair styling, skin care, sun care, oral care, or color cosmetic composition.

16. The hair care composition according to claim 15 that further comprises a solvent selected from the group consisting of: ethanol, dimethyl ether, water, and combinations thereof.

17. The hair care composition according to claim 15 having a product form that is a spray, mist, gel, tonic, or mousse.

18. The hair care composition according to claim 15 having up to 10% (w/w total) of said branched polymer.

19. The hair care composition according to claim 15 that provides a curl retention benefit.

20. The hair care product according to claim 15 that provides less on-comb or less on-hair flaking than an equivalent hair care composition made with completely linear PVP K-30, PVP K-60, or PVP K-90.

21. The oral care composition according to claim 15 that is a toothpaste, a mouthwash, a mouth rinse, a denture adhesive, a tooth whitener, a tooth stain remover, a tooth stain preventer, an anti-calculus composition, an oral gel, an anti-gingivitis, a gum, or a breath enhancing composition.
